(19) United States
(12) Patent Application Publication
(10) Pub. No.: US 2018/0161298 A1
(43) Pub. Date: Jun. 14, 2018

(54) TREATMENT OF AUTOPHAGY-RELATED DISORDERS

(71) Applicant: STC.UNM, Albuquerque, NM (US)

(72) Inventors: Vojo P. Deretic, Placitas, NM (US); Michael Mandell, Albuquerque, NM (US)

(21) Appl. No.: 15/813,438

(22) Filed: Nov. 15, 2017

Related U.S. Application Data
(63) Continuation of application No. 14/898,062, filed on Dec. 11, 2015, now abandoned.
(60) Provisional application No. 61/835,227, filed on Jun. 14, 2013.
(60) Provisional application No. 61/835,255, filed on Jun. 14, 2013.

(51) Int. Cl. A61K 31/201; C12N 9/00; A61K 38/53; C12Q 1/44; C12Q 1/61; A61K 45/06

(52) U.S. Cl. CPC A61K 31/201; C12N 9/93; G01N 2500/20; C12Y 603/02; A61K 38/53; A61K 45/06; C12Q 1/44; C12Q 1/61; G01N 2800/7028; G01N 2333/92; G01N 2405/00; G01N 2405/02

(57) ABSTRACT

The present invention relates to the use of neutral lipids, including triglycerides, diglycerides and monoglycerides which may be used to increase neutral lipids (lipid stores and/or lipid droplets) and neutral lipid stores in order to regulate (in particular, induce) autophagy and treat and/or prevent autophagy related disease states and/or conditions. In one embodiment, the invention relates to the use of neutral lipids and/or TRIM proteins which may be used to regulate (in particular, induce) autophagy, target autophagic substrates and treat and/or prevent autophagic disease states and/or conditions.

### CROSS REFERENCE TO RELATED APPLICATIONS

This application claims priority from U.S. Provisional Patent Application Ser. No. 61/835,227, filed Jun. 14, 2013 and entitled “Use of Triglycerides and Neutral Lipids to Enhance Lipid Droplets and Autophagy, and Treat Autophagic Disease States and Conditions”, and U.S. Provisional Patent Application Ser. No. 61/835,255, filed Jun. 14, 2013 and entitled “Use of TRIM proteins to regulate autophagy, target autophagic substrates by direct recognition and treat autophagic disease states and conditions”. These provisional applications and their complete contents are hereby incorporated by reference in their entirety.

### STATEMENT REGARDING FEDERAL FUNDING

This invention was made with government support under grants R01 AI042999 and R01 AI111935 awarded by the National Institutes of Health (NIH). The government has certain rights in the invention.

### FIELD OF THE INVENTION

The present invention relates to the use autophagy modulators. Neutral lipids, including triglycerides, diglycerides and monoglycerides are autophagy modulators which can be used to increase neutral lipids (lipid stores and/or lipid droplets) and neutral lipid stores in order to regulate (in particular, induce) autophagy and treat and/or prevent autophagy related disease states and/or conditions. TRIM proteins are also autophagy modulators which can be used to regulate (in particular, induce) autophagy, target autophagic substrates and treat and/or prevent autophagic disease states and/or conditions. The neutral lipids and TRIM proteins described herein can be used alone, together, or in combination with another autophagy modulator compound in order to induce autophagy and treat and/or an autophagy related disease.

### BACKGROUND OF THE INVENTION

The sense stricto autophagy (macroautophagy) is a fundamental biological process [1]. Dysfunctional autophagy has been linked to human pathologies in aging, cancer, neurodegeneration, myopathies, metabolic disorders, infections and inflammatory diseases [2-4]. Autophagy degrades bulk cytosol during starvation, surplus or damaged organelles (e.g. depolarized mitochondria [5], lipid droplets (LD) [6], etc.), toxic protein aggregates [4] and intracellular pathogens [3].

The specialized committed step in mammalian autophagy is the induction and nucleation of a membranous precursor called phagophore that expands and closes into an emblematic structure, the double membrane autophagosome. The elongation of the phagophore depends on Atg5-Atg12/Atg16L1 complex which acts as an E3 ligase for conjugation of the mammalian orthologs of Atg8 (e.g. LC3) to the phosphatidylethanolamine [1, 7]. Autophagosomes sequester portions of the cytoplasm or specific targets and fuse with lysosomes [8] to digest the captured cargo. Each of these steps involves the hierarchical activity of Atg (Autophagy-related) proteins [1, 7]. The formation of the phagophore requires mammalian orthologs of Atg1 (Ulk1/2) and the class III phosphoinositide 3-kinase (PI3K) Vps34 complexed with the mammalian ortholog of Atg6 (Beclin-1) to generate phosphatidylinositol 3-phosphate (PI3P) [9]. A PIP-binding protein, DFCP1, marks omegasome structures derived from the ER that serve as precursors [10] to the ER-derived autophagosomes [11, 12]. PI3P also recruits mammalian orthologs of Atg18 (WIPI1 and WIPI2) contributing to the subsequent steps in phagophore formation [13, 14].

The source of the autophagosome membrane remains an important question in the field of autophagy [15-17]. Several compartments, including the endoplasmic reticulum (ER) [11, 12], mitochondria [18], mitochondria-ER contact sites [17], Golgi apparatus [19, 20], and the plasma membrane [21], have been implicated as contributing sources to autophagosomal membranes. Given that autophagy is a process requiring intense membrane remodeling and consumption, and thus could impinge on the functionality of the organelles proposed to be the membrane sources, we wondered whether the cell may utilize its neutral lipid stores to supplement autophagosomal membrane formation.

LDs are dynamic organelles that represent a major depot of cellular neutral lipids such as triglycerides (TG) [22, 23]. In addition to their role as substrates for lipophagy [6], the process known as autophagic degradation of LDs, we considered an alternative possibility that LDs might serve as organelles whereby TO stores could be mobilized into phospholipids necessary for autophagosomal membrane formation and growth. To address this question, TG mobilizing enzymes were screened for their capacity to affect autophagic pathway. We found that PNPLA5, a factor that possesses a lipase activity with TGs as substrates [24], was needed for optimal autophagy initiation. We present evidence that LDs and TGs via PNPLA5 contribute lipid intermediates facilitating autophagosome membrane biogenesis.

The tripartite motif (TRIM) family of proteins (Jefferies at al., 2011; Kawai and Akira, 2011; Ozato et al., 2008; Reymond et al., 2001b) thus far has not been tested for potential connections with autophagy. TRIMs include more than 70 members in humans and typically consist of time motifs: a N-terminal RING domain with ubiquitin E3 ligase activity, a B-box, and a coiled-coil domain, as well as a variable C-terminal domain, which has a role in substrate binding (Kawai and Akira, 2011.). In addition to demonstrating the impact of neutral lipids on autophagy, we also show that TRIM5α is involved in autophagy induction and interacts with the key autophagy factors Beclin 1 and ULK1. TRIM5α promotes Beclin 1 release from inhibitory complexes containing Bcl-2 and TAB2. We furthermore identify a second role for TRIM5α in autophagy: that of acting as an ubiquitin-independent selective autophagy adaptor involved in delivery of its cargo to autophagosomes for degradation.

### SUMMARY OF THE INVENTION

In one embodiment, the present invention provides a method of modulating autophagy in a subject who suffers from an autophagy-related disorder, e.g. a cancer, Alzheimer's disease, Parkinson's disease, various ataxias, chronic inflammatory diseases (e.g. inflammatory bowel disease, Crohn's disease, rheumatoid arthritis, lupus, multiple sclerosis, chronic obstructive pulmony disease/COPD, pulmonary fibrosis, cystic fibrosis, Sjogren's disease), diabetes and metabolic syndrome, muscle degeneration and atrophy, frailty in aging, stroke and spinal cord injury, arteriosclerosis, infectious diseases (HIV I and II, HBV, HCV, including secondary disease states or conditions associated with infectious diseases, including AIDS) and tuberculosis.

An autophagy-modulating agent such as a neutral lipid and/or TRIM protein may be administered to a subject who suffers from an autophagy-related disorder in order to modulate autophagy, i.e., to up-regulate autophagy or, if the subject suffers from certain autophagy-related disorders (e.g. cancer), to down-regulate autophagy.

While not wishing to be bound by any theory, we believe that administration of uncharged or weakly charged (“neutral”) lipids (e.g. lipids selected from the group consisting of triglycerides, diglycerides, monoglycerides, glycolated mono- or diacylglycerdies, dolichol, polyprenol, polyprenal or very long chain fatty acids) to a subject suffering from an autophagy-related disorder enhances lipid storage and promotes lipid droplet formation in relevant cells of the subject, thereby enhancing autophagy and treating symptoms of the disorder.

In one embodiment, a subject who suffers from an autophagy-related disorder is treated with a neutral lipid mono-therapy, a TRIM protein monotherapy as described herein, or a co-therapy regimen in which the subject is administered both a neutral lipid and TRIM protein. In each instance, these methods may optionally include another autophagy regulating compound as described herein. The subject may also be treated with compositions such as L-carnitine, acetyl-L-carnitine or other agents that are involved in lipid metabolism and which are implicated in the breakdown of fat tissues and/or cellulite. Additional bioactive agents e.g., an anticancer agent, an antibiotic, an anti-tuberculosis agent, antiviral agents such as an anti-HIV agent, anti-HBV agent or an anti-HCV agent, among others, may also be included in methods according to the present invention.

The invention also provides screening methods to determine whether a composition will enhance lipid stores and/or lipid droplets in individuals. The present invention also relates to diagnostic methods whereby TRIM protein levels are assessed in an individual to determine if a neutral lipid should be administered to a patient in order to enhance autophagy, thereby treating or reducing the likelihood of an autophagy related disease state.

TRIM proteins which are useful in the present invention, include, but are not limited to, TRIM5a, TRIM1, TRIM6, TRIM10, TRIM17, TRIM22, TRIM41, TRIM55, TRIM72 and TRIM76, among others (including TRIM 1, TRIM2, TRIM23, TRIM26, TRIM28, TRIM31, TRIM 32, TRIM33, TRIM38, TRIM42, TRIM44, TRIM45, TRIM49, TRIM50, TRIM51, TRIM58, TRIM59, TRIM65, TRIM68, TRIM73, TRIM74 and TRIM76 and mixtures thereof.

In one embodiment, the invention provides a method of treating a subject who suffers from a cancer selected from the group consisting of Stage IV small cell lung cancer, ductal carcinoma in situ, relapsed and refractory multiple myeloma, brain metastases from solid tumors, breast cancer, primary renal cell carcinoma, previously treated renal cell carcinoma, pancreatic cancer, Stage IIb or III adenocarcinoma of the pancreas, non-small cell lung cancer, recurrent advanced non-small cell lung cancer, advanced/recurrent non-small cell lung cancer, metastatic breast cancer, colorectal cancer, metastatic colorectal cancer, unspecified adult solid tumor, al I-antitrypsin deficiency liver cirrhosis, amyotrophic lateral sclerosis and lymphangioleiomyomatosis by administering to the subject a pharmaceutically-effective amount of at least one neutral lipid selected from the group consisting of triglycerides, diglycerides, monoglycerides, glycolated mono- or diacylglycerdies, dolichol, polyprenol, polyprenal and very long chain fatty acids and an autophagy-modulating anti-cancer agent selected from the group consisting of chloroquine, hydrochloroquine, carbamazepine, lithium carbonate and trehalose.

In certain embodiments, chloroquine is combined with cyclophosphamide and velcade or is administered together with whole-brain irradiation; and hydroxychloroquine is combined with one or more compositions selected from the group consisting of the mTOR inhibitor RAD001, gemcitabine, carboplatin, paclitaxel, and bevacizumab, ixabepilone, temsirolimus, sunitinib, vorinostat, MK2206, ABT-263 or abiraterone, docetaxel, sirolimus, vorinostat and bortezomib.

Pharmaceutical compositions of the invention comprise an effective amount of at least one autophagy-modulating composition (e.g. a neutral lipid and/or a TRIM protein), optionally in combination with an effective amount of another active ingredient such as L-carnitine, Acetyl-L-carnitine or other lipid metabolism lipolysis enhancing agents and/or additional bioactive agents (e.g., an anticancer agent, an antibiotic, an anti-tuberculosis agent, antiviral agents such as an anti-HIV agent, anti-HBV agent or an anti-HCV agent, among others).

Methods of treatment and pharmaceutical compositions of the invention may also entail the administration of additional autophagy modulators selected from the group consisting of flubendazole, hexachlorophene, propidium iodide, bepridil, clomiphene citrate (Z,E), GBR 12909, propafenone, metixene, dipivefrin, fluvoxamine, dicyclomine, dimethisoquin, ticlopidine, memantine, bromhexine, norcyclobenzaprine, diperodon, nortriptyline, tetrachlorisophthalonitrile, phenylmercuric acetate, benzethonium, niclosamide, monensin, bromperidol, levobunolol, dehydroisoandosterone 3-acetate, sertraline, tamoxifen, reserpine, hexachlorophene, dipyridamole, harmaline, prazosin, lidoflazine, thiethylperazine, dextromethorphan, desipramine, mebendazole, canrenone, chlorprothixene, maprotiline, homochlorcyclizine, loperamide, nicardipine, dexfenfluramine, nilvadipine, dosulepin, biperiden, denatonium, etomidate, toremifene, tomoxetine, clorgyline, zotepine, beta-escin, tridihexethyl, ceftazidime, methoxy-6-harmalan, melengestrol, albendazole, rimantadine, chlorpromazine, pergolide, cloperastine, prednicarbate, haloperidol, clotrimazole, nitrofural, iopanoic acid, naftopidil, methimazole, trimeprazine, ethoxyquin, clocortolone, doxycycline, pirlindole mesylate, doxazosin, deptropine, nocodazole, scopolamine, oxybenzone, halcinonide, oxybutynin, miconazole, clomipramine, cyproheptadine, doxepin, dyclonine, salbutamol, flavoxate, amoxapine, fenofibrate, pimethixene, a pharmaceutically acceptable salt thereof and mixtures thereof.

The invention also provides diagnostic methods in which sample lipid stores and/or lipid droplets are obtained from a subject and assessed to determine if a neutral lipid should be administered to the subject in order to enhance autophagy, thereby treating or reducing the likelihood of an autophagy related disease state.

These and other aspects of the invention are described further in the Detailed Description of the Invention.

### BRIEF DESCRIPTION OF THE FIGURES

FIG. 1 illustrates that preformed lipid droplets enhance starvation-induced autophagy, as determined in the experiment(s) of Example 1.

FIG. 2 shows that early autophagic markers colocalize with LDs, as determined in the experiment(s) of Example 1.

FIG. 3 shows that lipid droplets are consumed during autophagic induction independently of autophagosomal closure and autophagic maturation, as determined in the experiment(s) of Example 1.

FIG. 4 illustrates a screen for triglyceride metabolism factors identifies PNPLA5, CPT1 and LPCAT2 as positive regulators of autophagy, as determined in the experiment(s) of Example 1.

FIG. 5 shows the co-localization between DAG, Atg16L1 and lipid droplets upon overexpression of mutant Atg4B^{C74A}, as determined in the experiment(s) of Example 1.

FIG. 6 shows that PNPLA5 is required for efficient autophagy of diverse autophagic substrates, as determined in the experiment(s) of Example 1.

FIG. 7 illustrates that lipid droplets contribute to autophagosome biogenesis, as determined in the experiment(s) of Example 1.

FIG. 8. Shows a flow chart of adipophagy treatment. An autophagy modulator as described herein is administered to a patient in need to breakdown fat tissue and/or cellulite alone, or combination with L-carnitine and/or Acetyl-L-carnitine. *Combine with L-carnitine to coordinate lipolysis with beta-oxidation in mitochondria (L-carnitine helps transport fatty acids into mitochondria). **Fat cellulite, cushions, etc.; visceral fat may need alternative modes of delivery.

Figure S1 shows the absence of WIPI co-localization with lipid droplets under basal conditions, as determined in the experiment(s) of Example 2.

Figure S2 shows the analysis of triglyceride mobilizing factors PNPLAs, Kennedy biosynthetic cycle and Lands remodeling cycle enzymes in lipid droplet contribution to the cellular autophagic capacity, as determined in the experiment(s) of Example 2.

Figure S3 shows imaging of DAG and Atg16L1 co-localization, as determined in the experiment(s) of Example 2.

Figure S4 shows that PNPLA5 knockdown inhibits lipid droplets consumption upon induction of autophagy by starvation, as determined in the experiment(s) of Example 2.

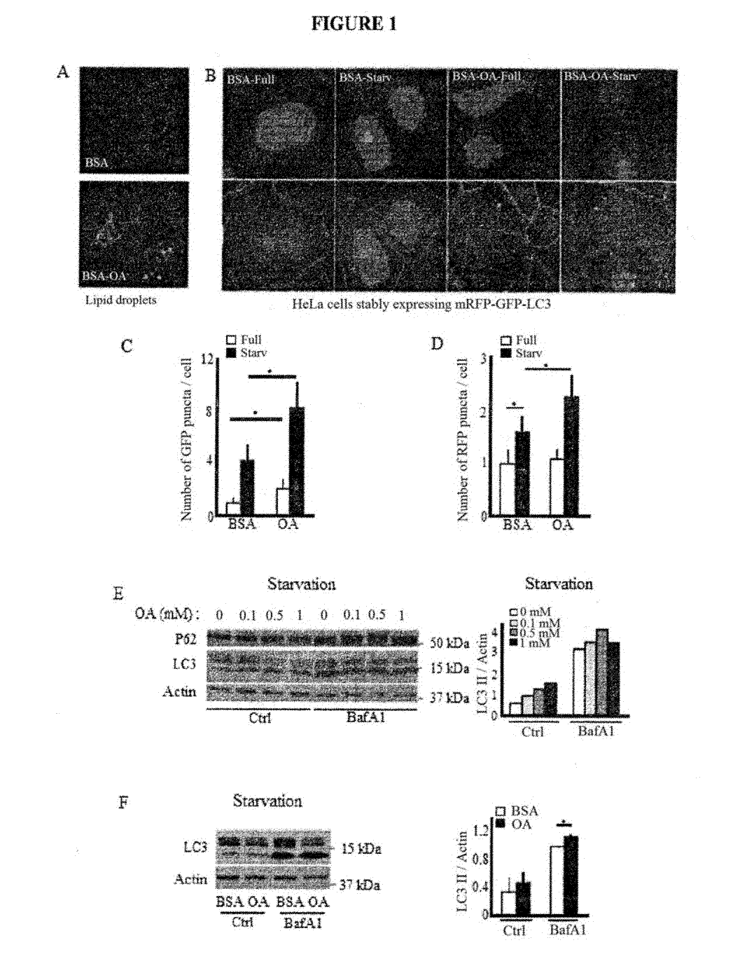

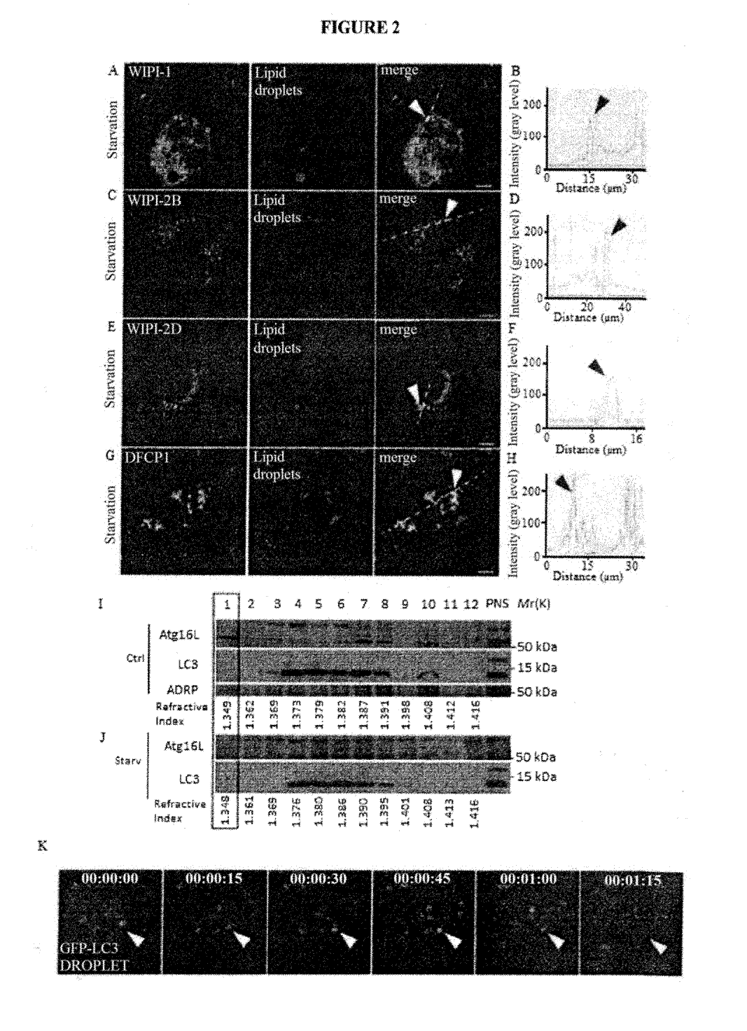

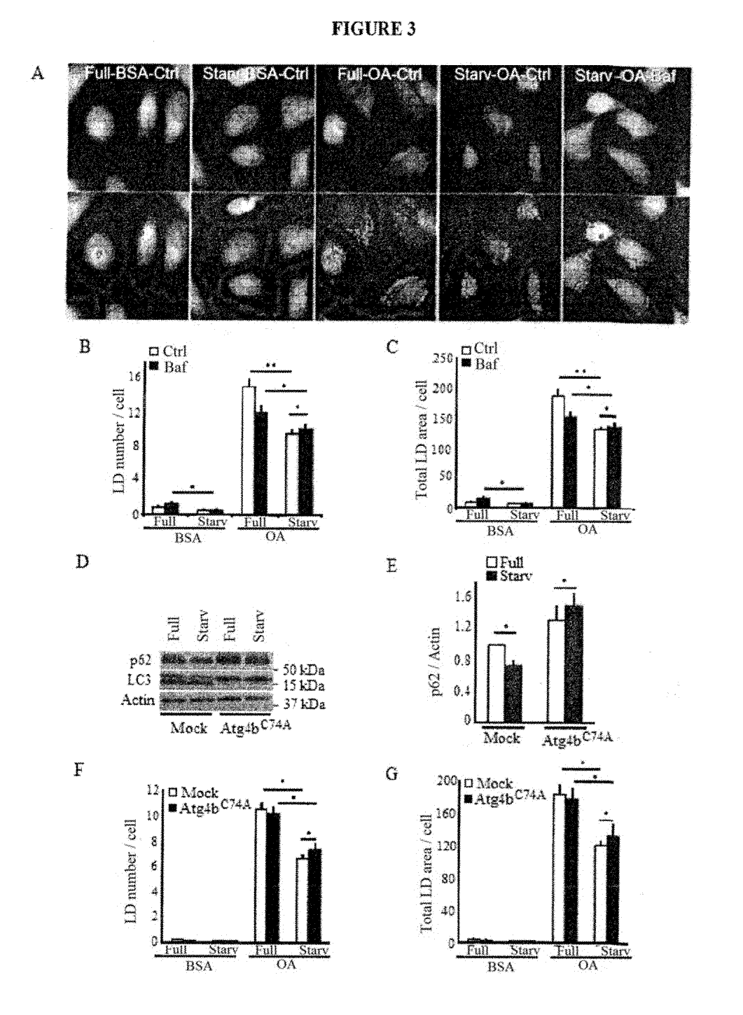

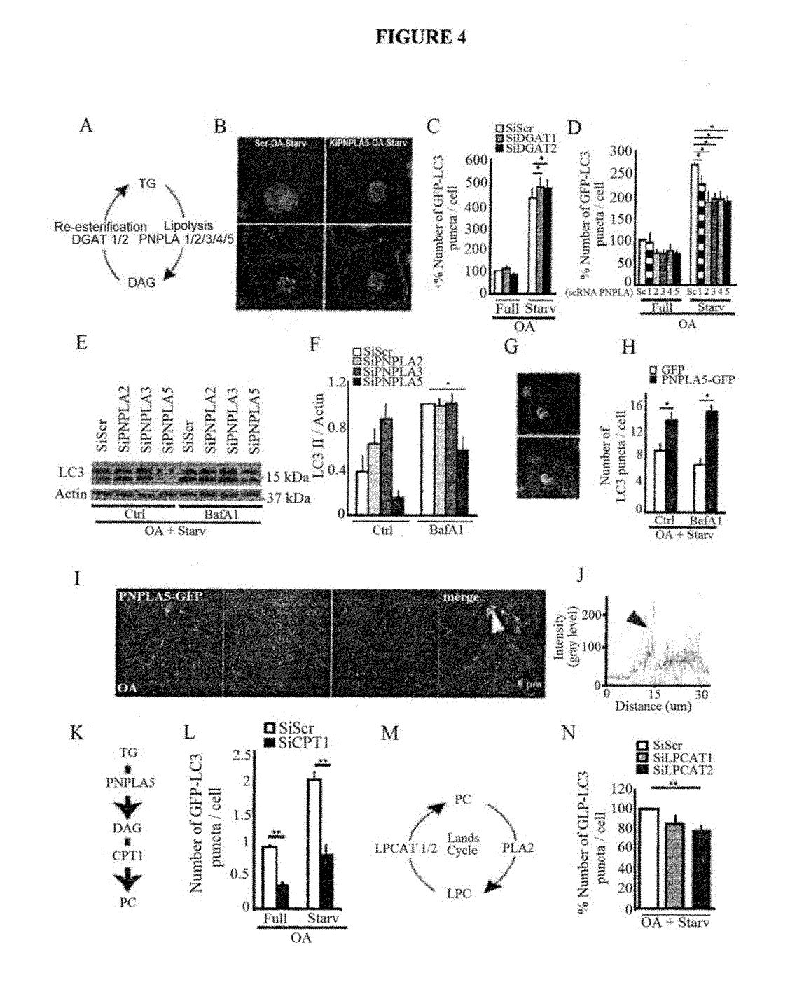

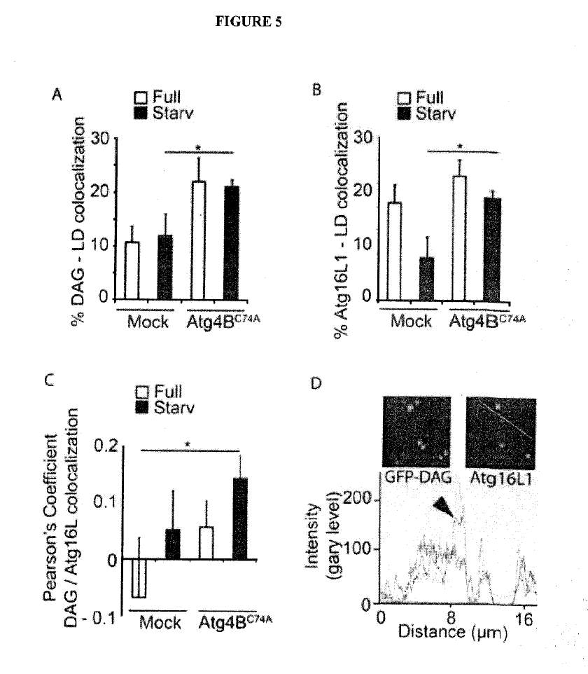

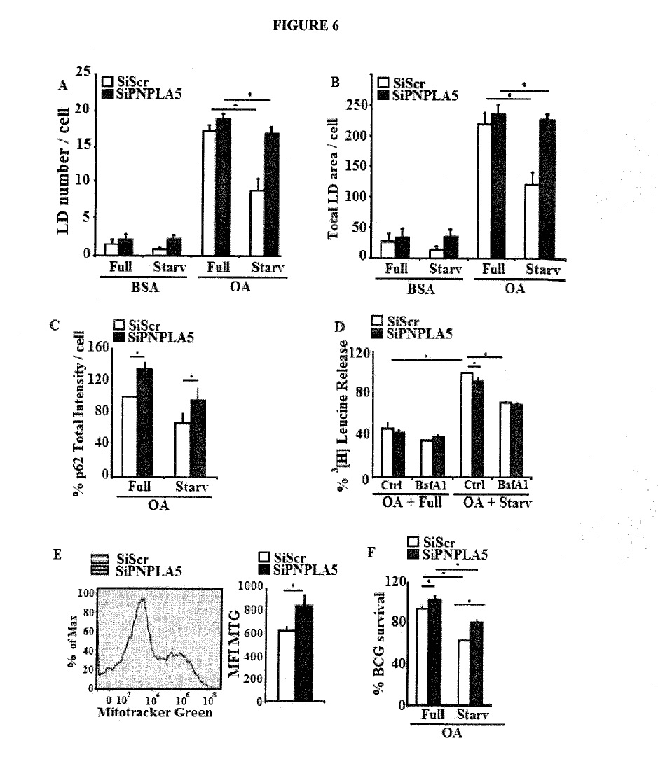

FIG. 1A illustrates that TRIM proteins regulate autophagy, as determined in the experiment(s) of Example 3.

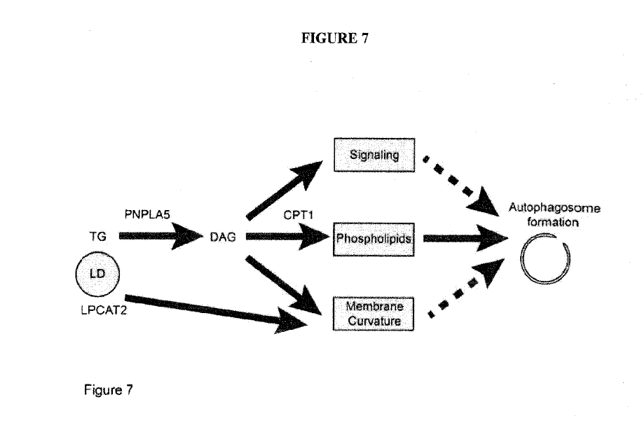

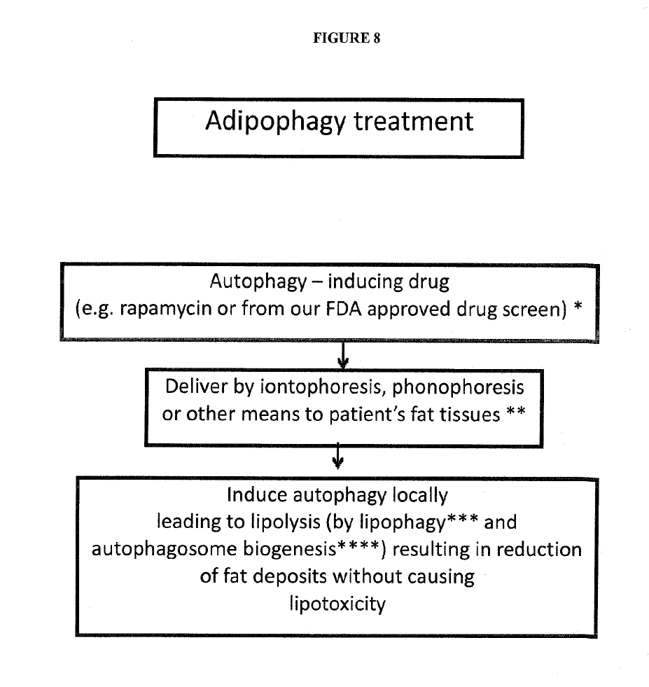

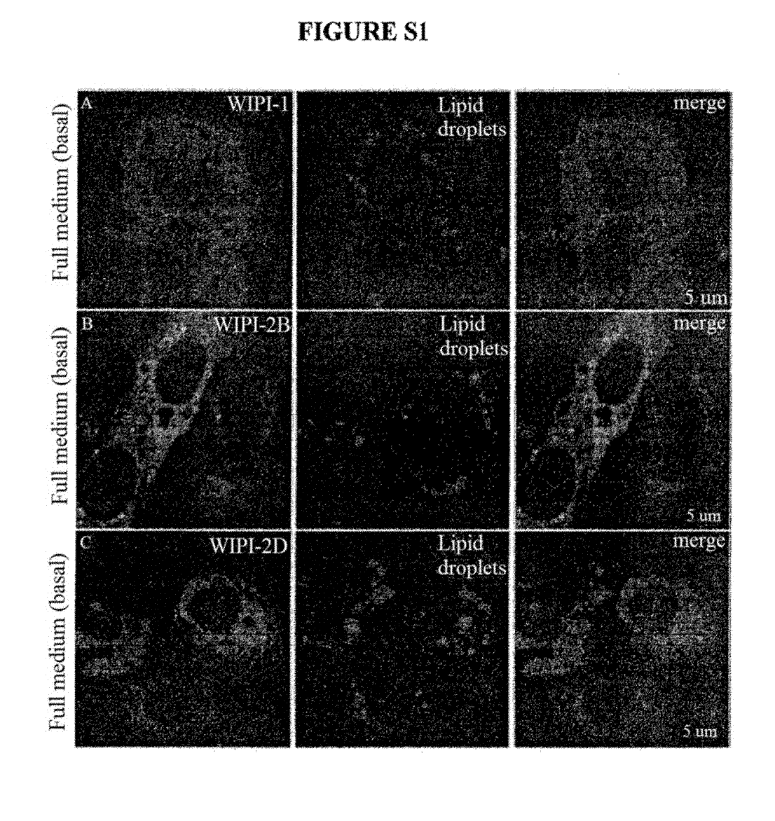

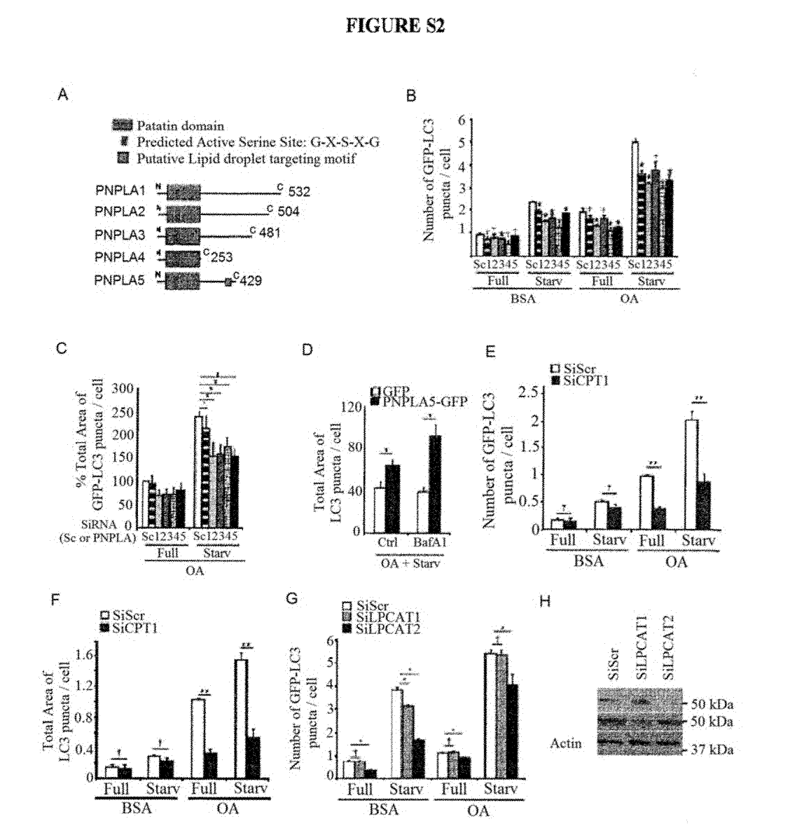

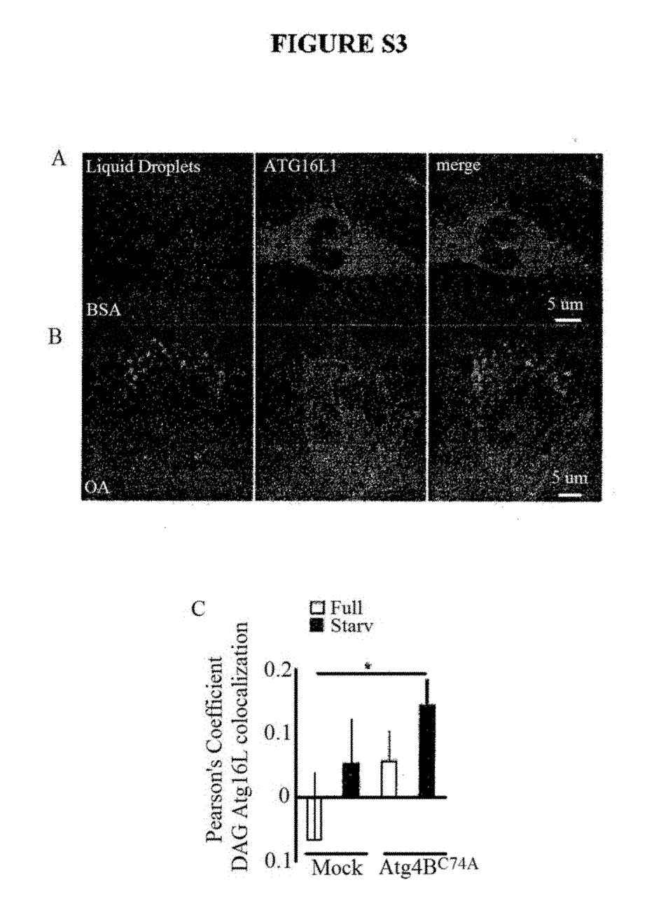

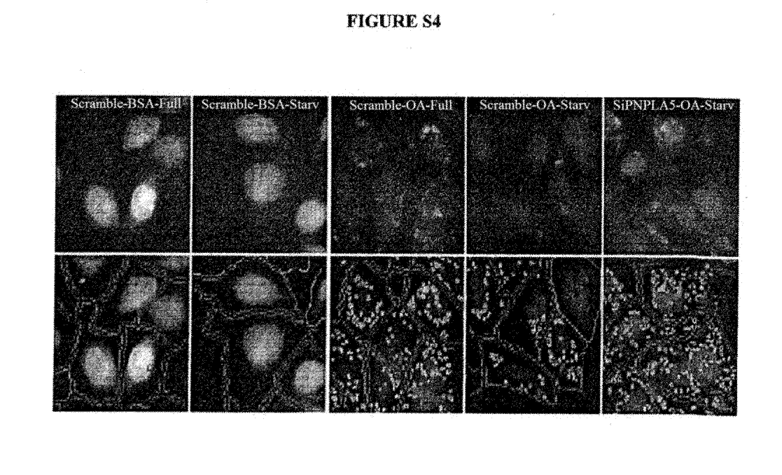

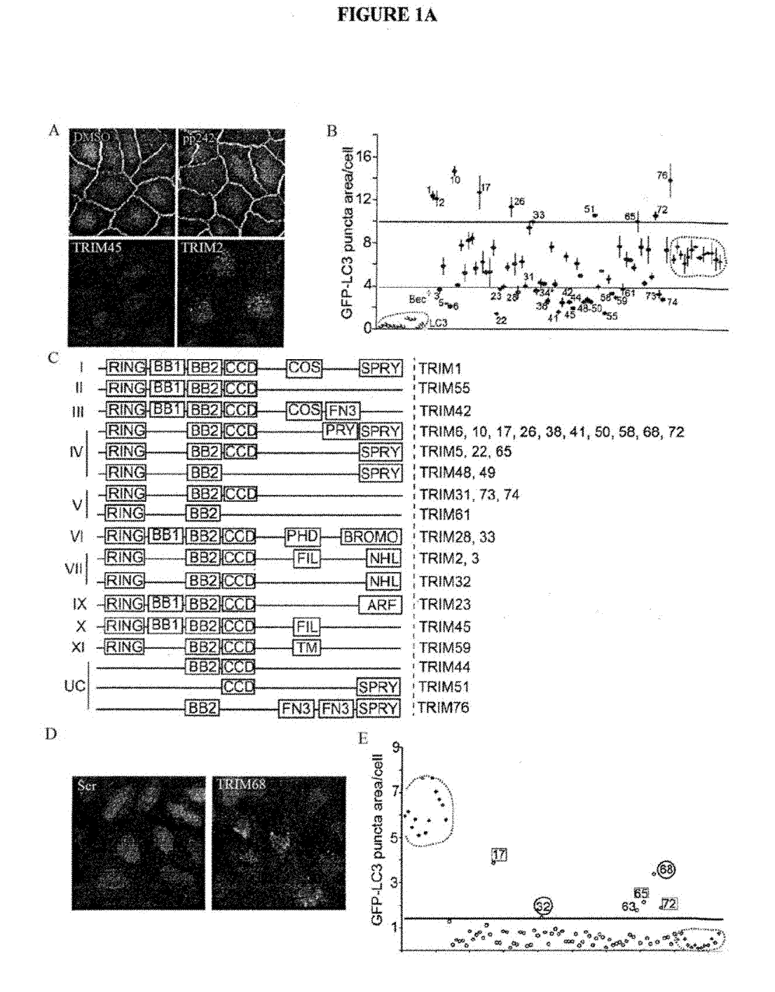

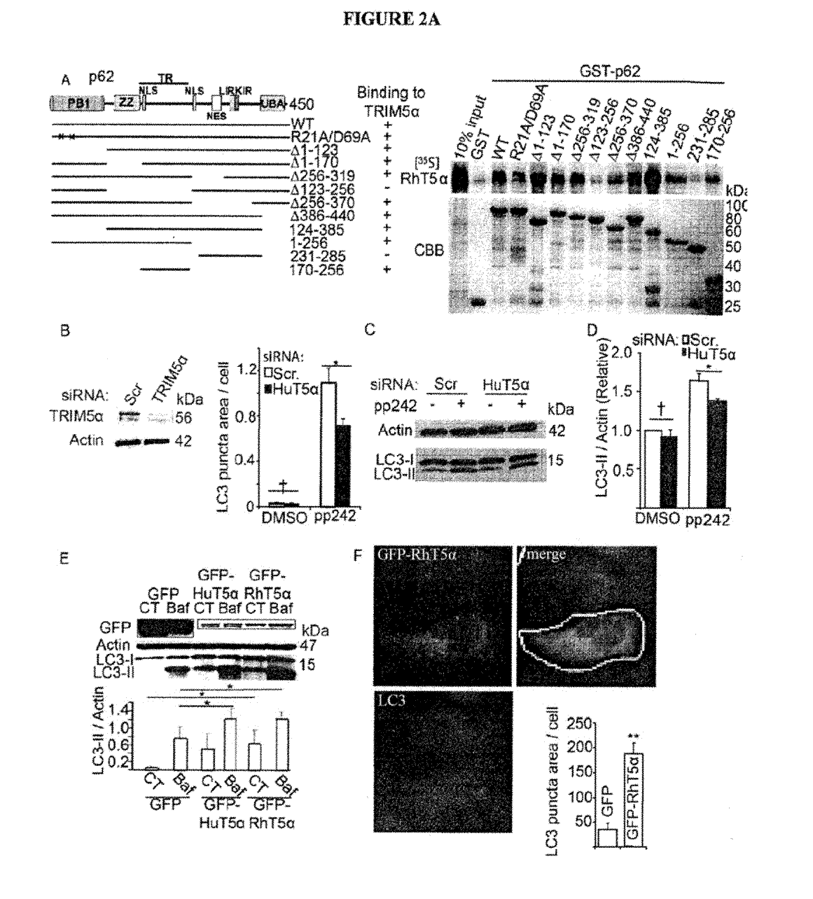

FIG. 2A illustrates that TRIM5α participates in autophagy induction, as determined in the experiment(s) of Example 3.

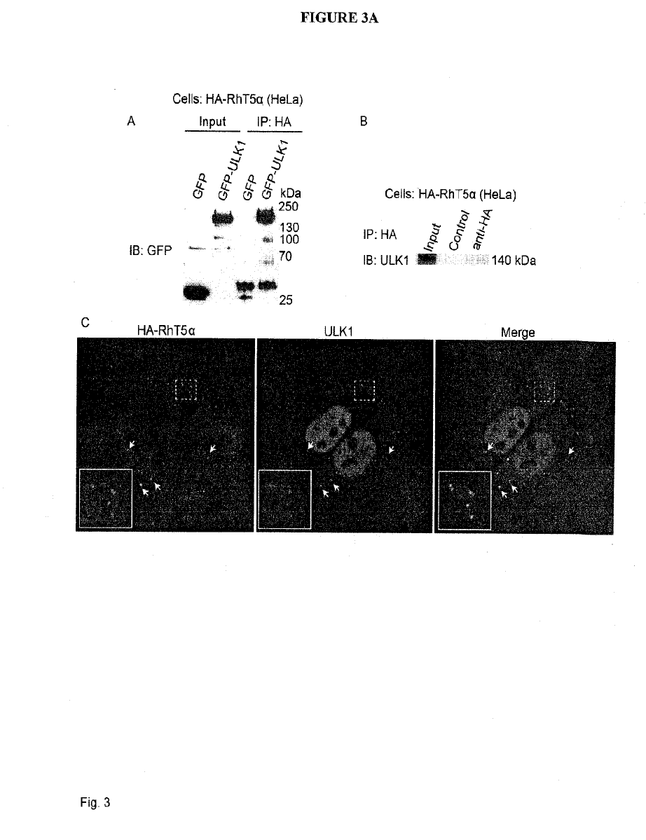

FIG. 3A illustrates that TRIM5α is in a complex with key autophagy regulator ULK1, as determined in the experiment(s) of Example 3.

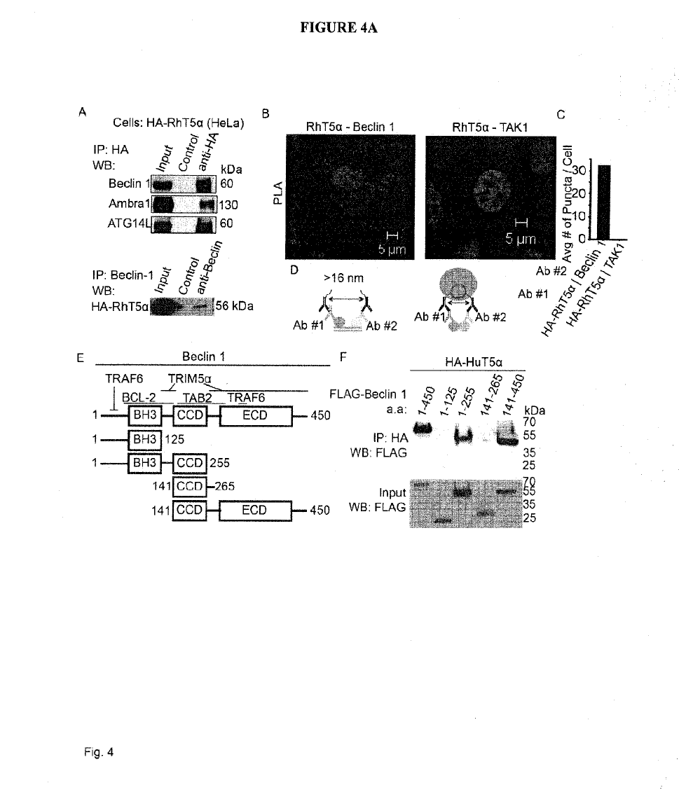

FIG. 4A illustrates that TRIM5a interacts with key autophagy factor Beclin 1, as determined in the experiment(s) of Example 3.

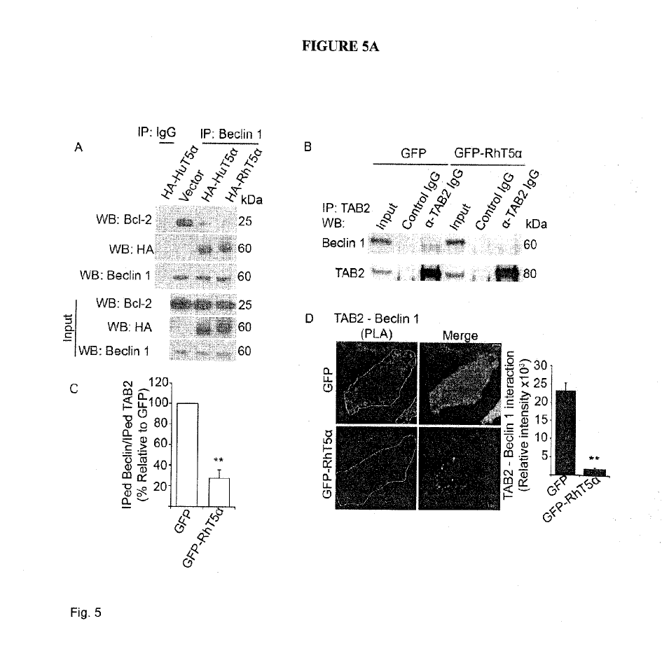

FIG. 5A illustrates that TRIM5a promotes release of Beclin 1 from negative regulators Bcl-2 and TAB2, as determined in the experiment(s) of Example 3.

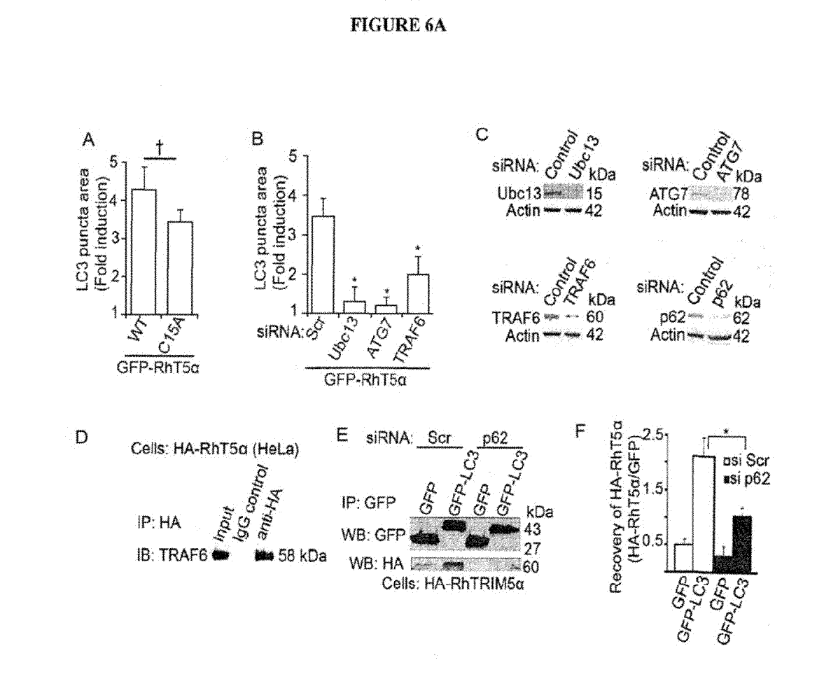

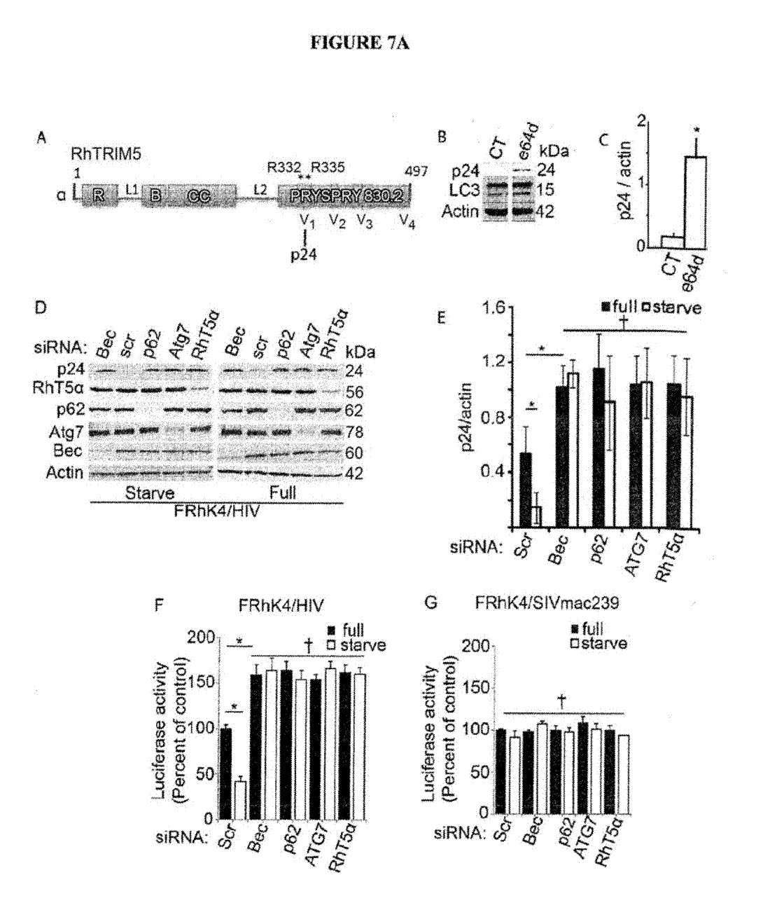

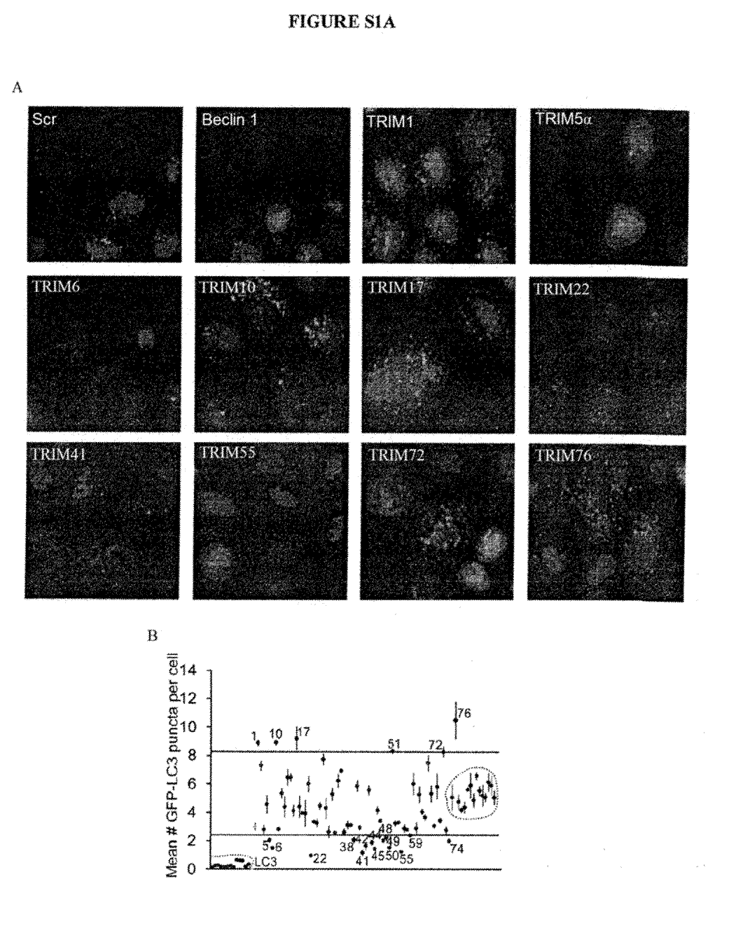

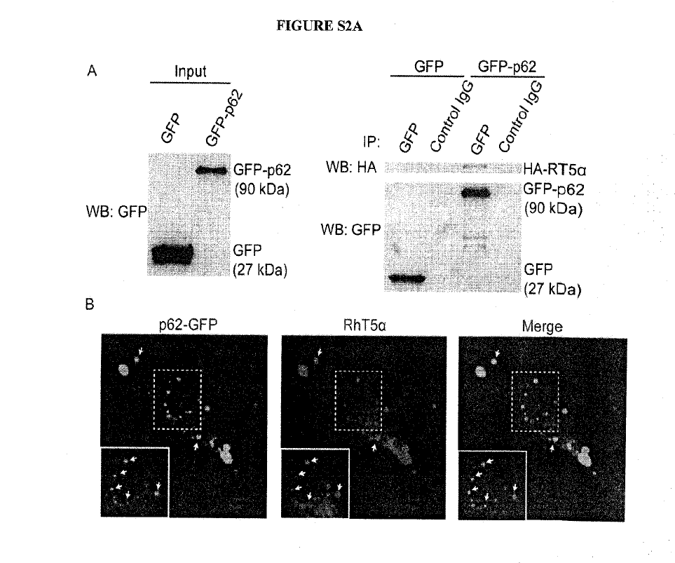

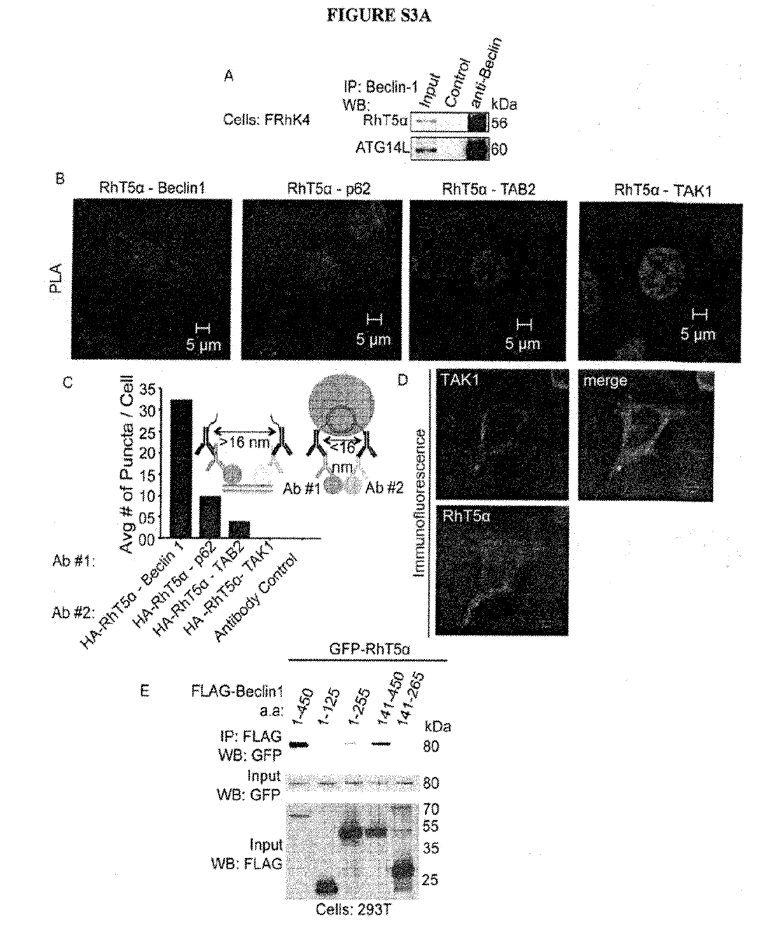

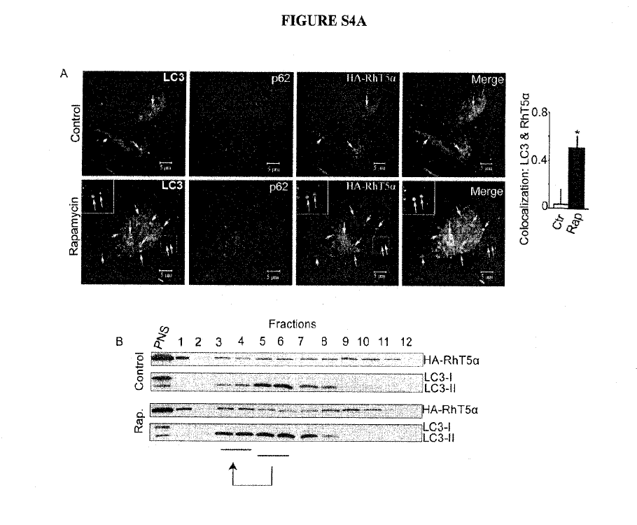

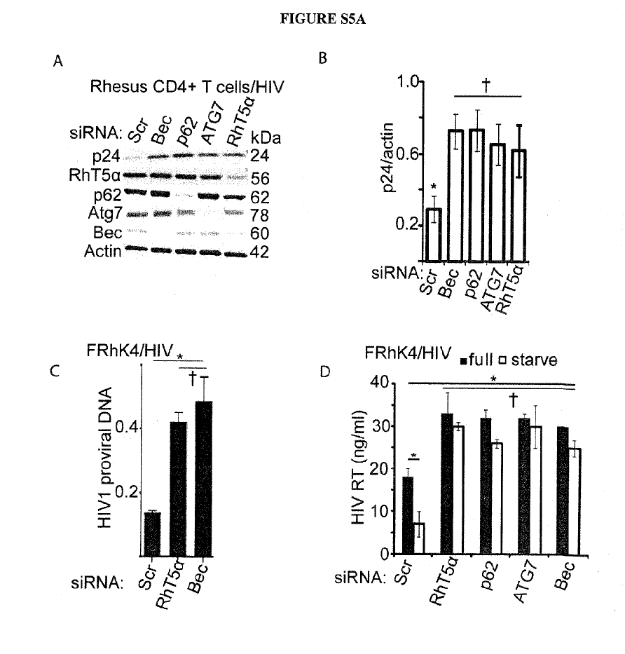

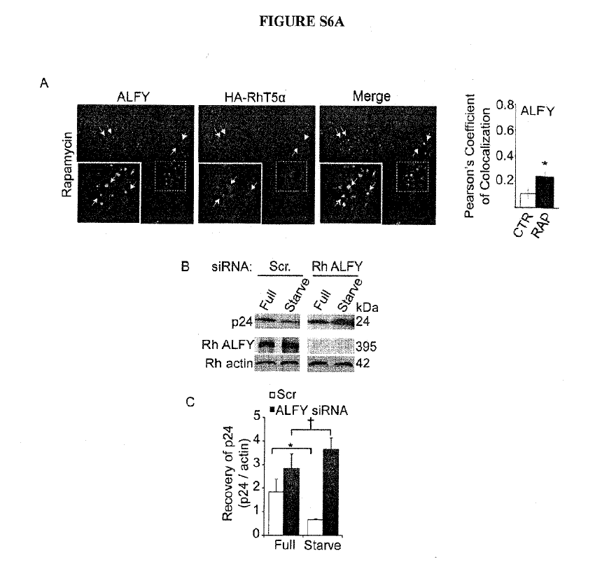

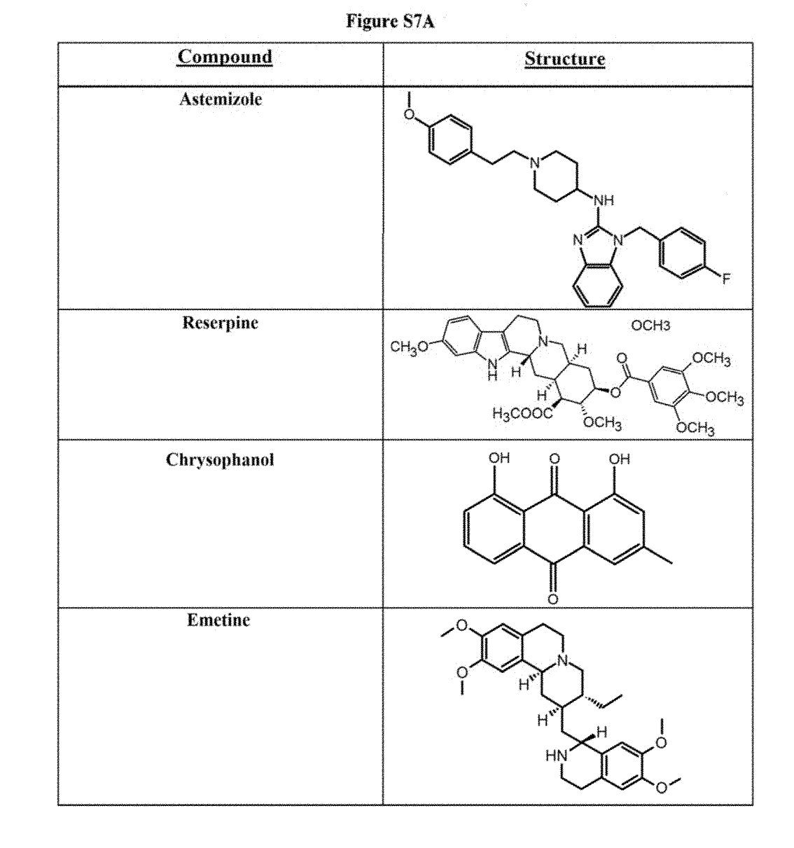

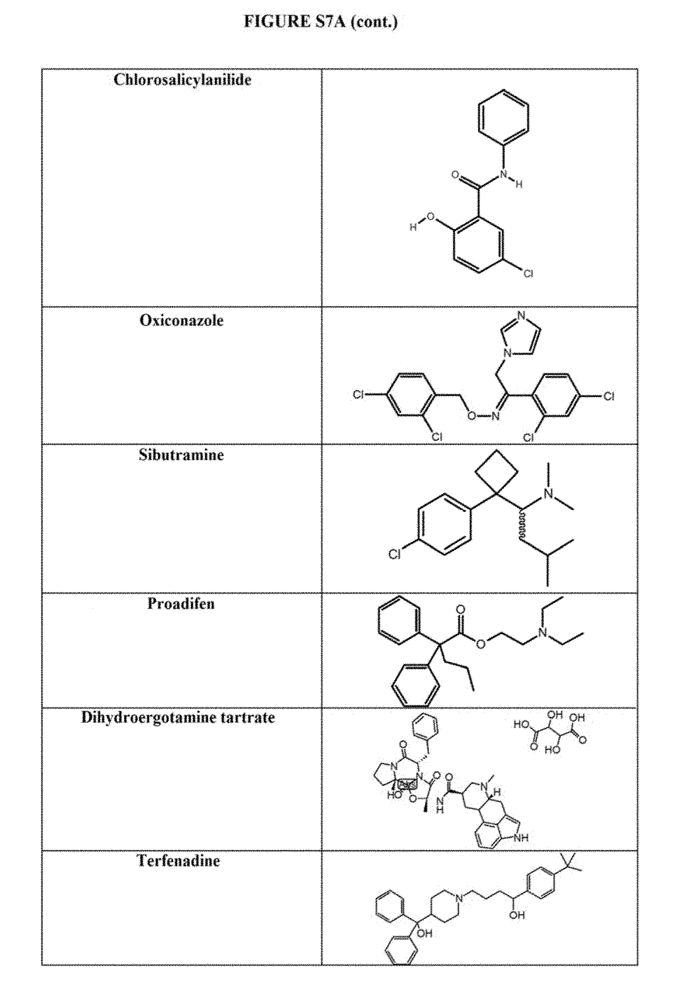

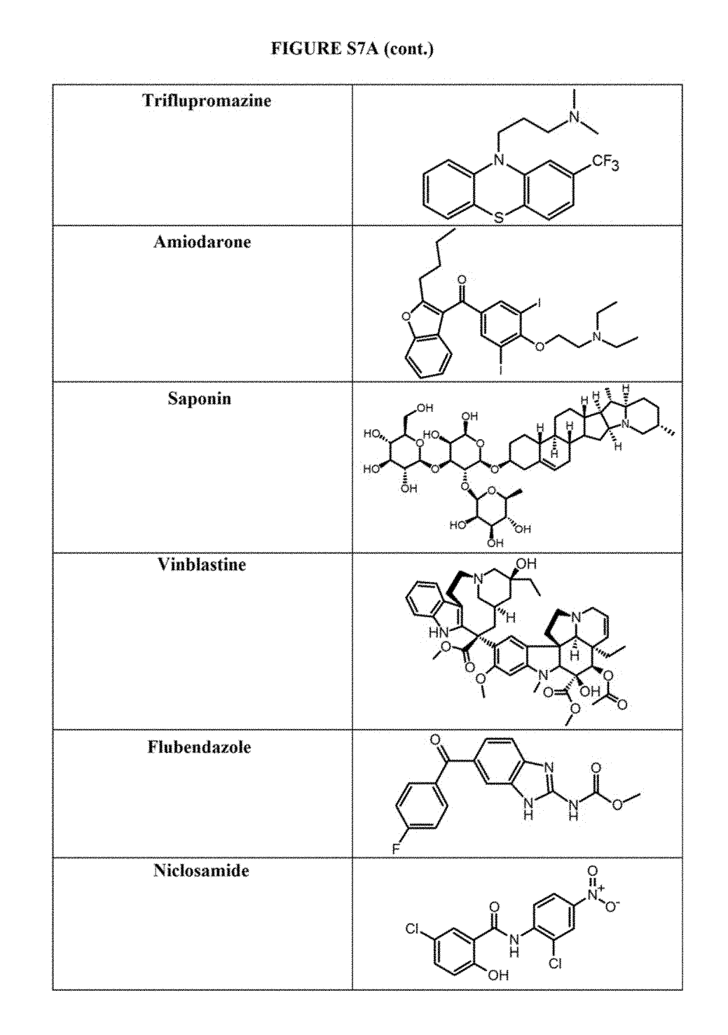

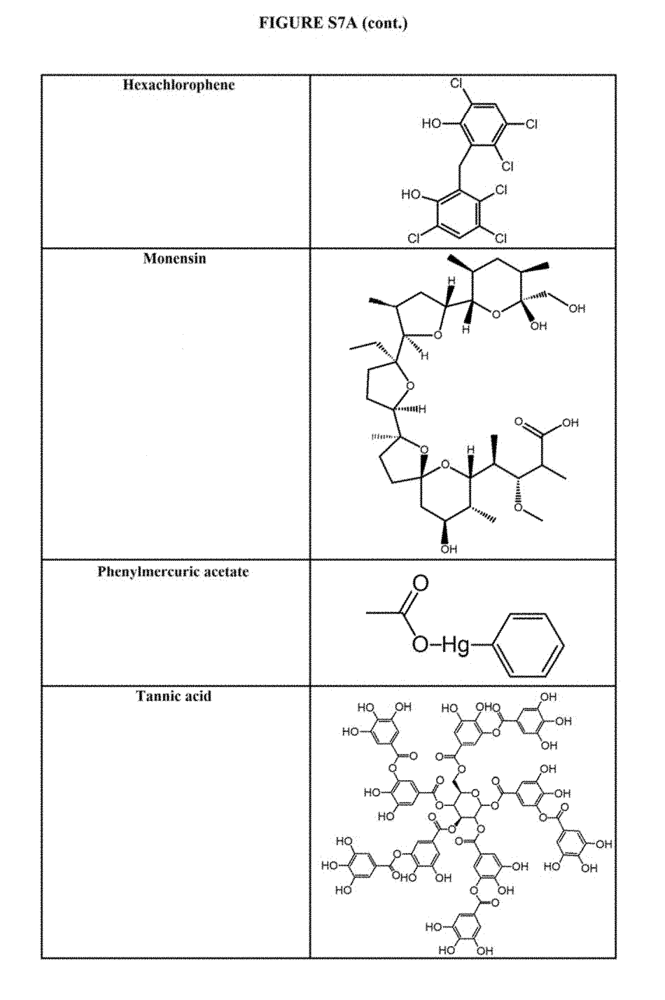

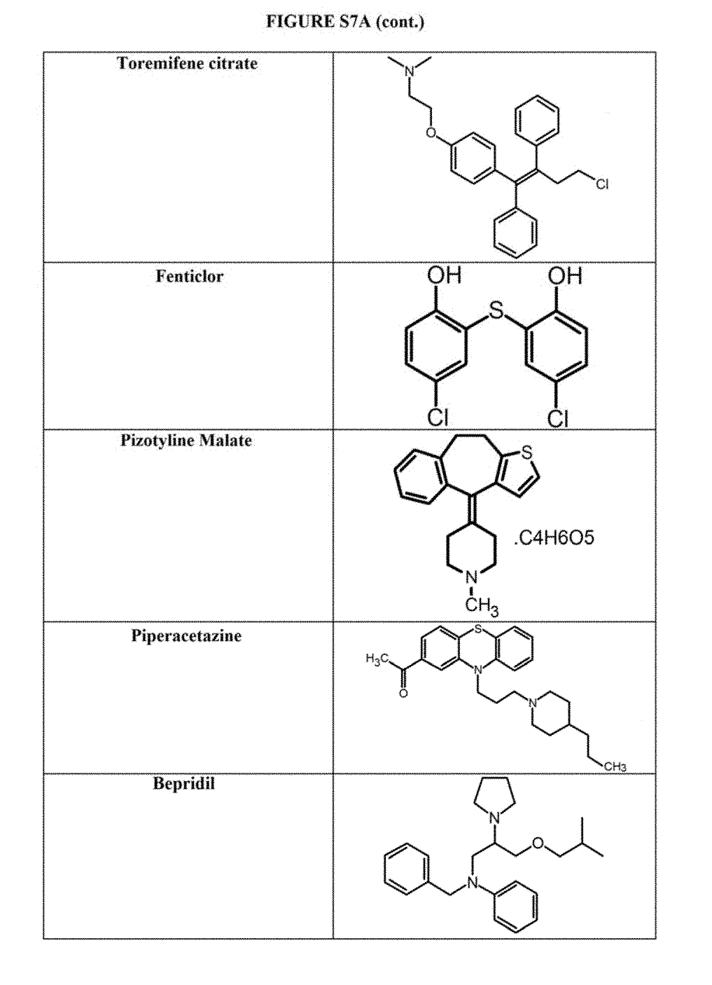

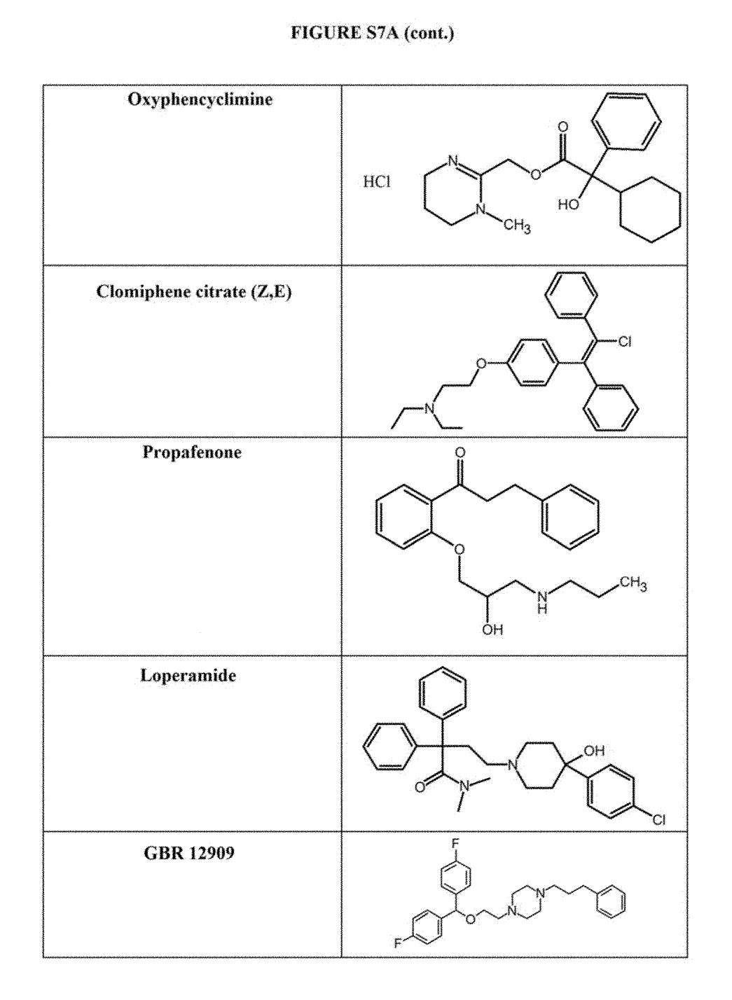

FIG. 6A illustrates the requirements for TRIM5α-induced autophagy and presence of TRAF6 and LC3 in TRIM5a complexes, as determined in the experiment(s) of Example 3.

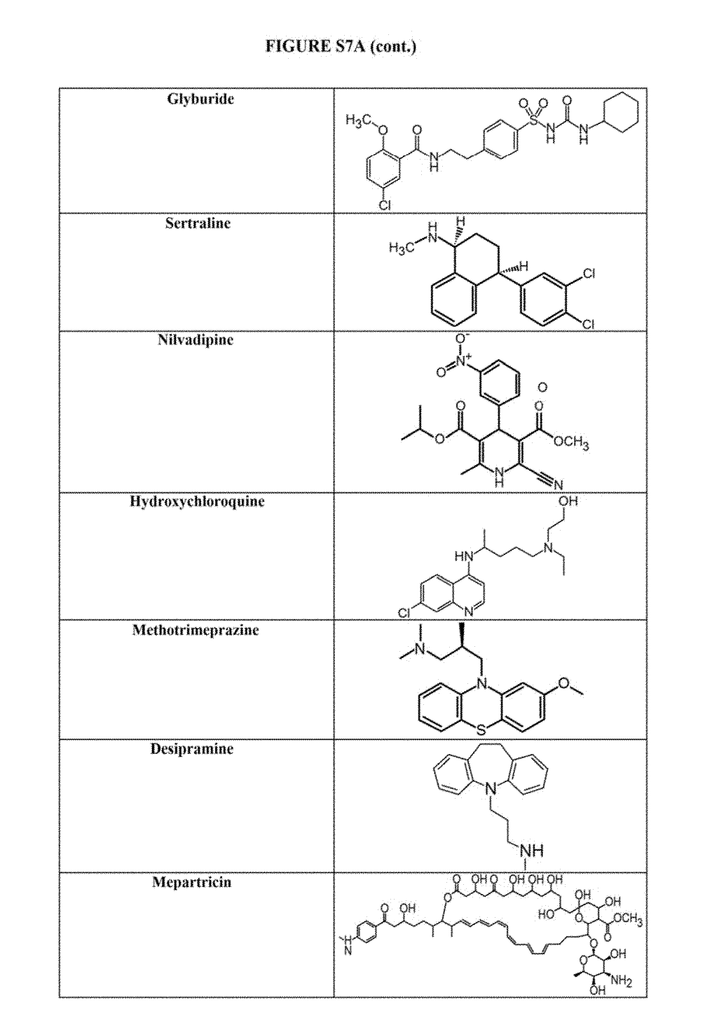

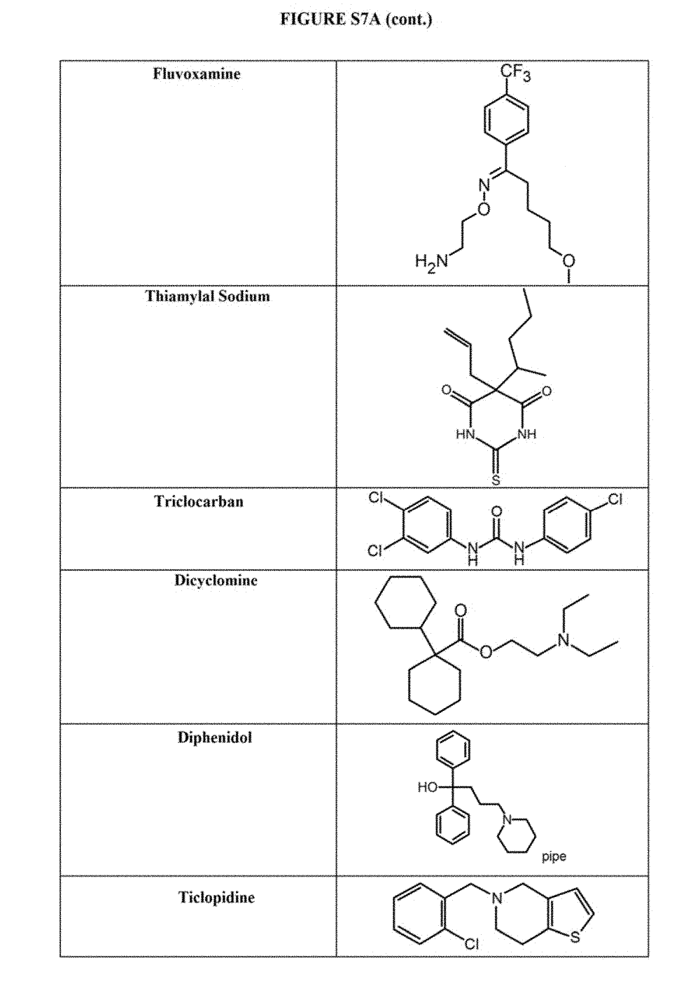

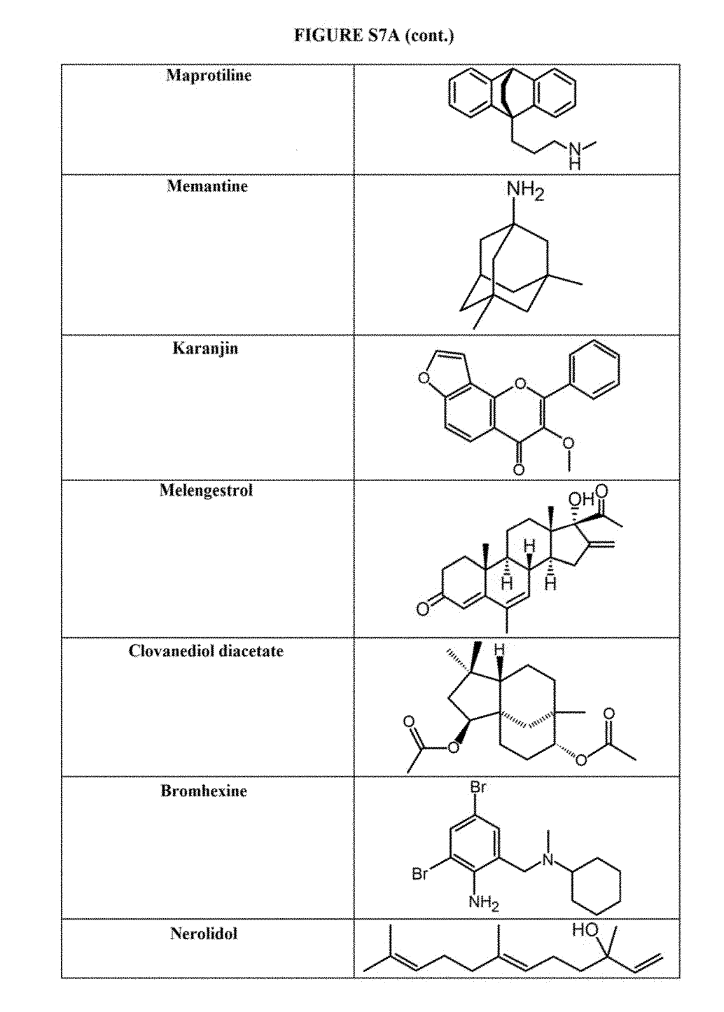

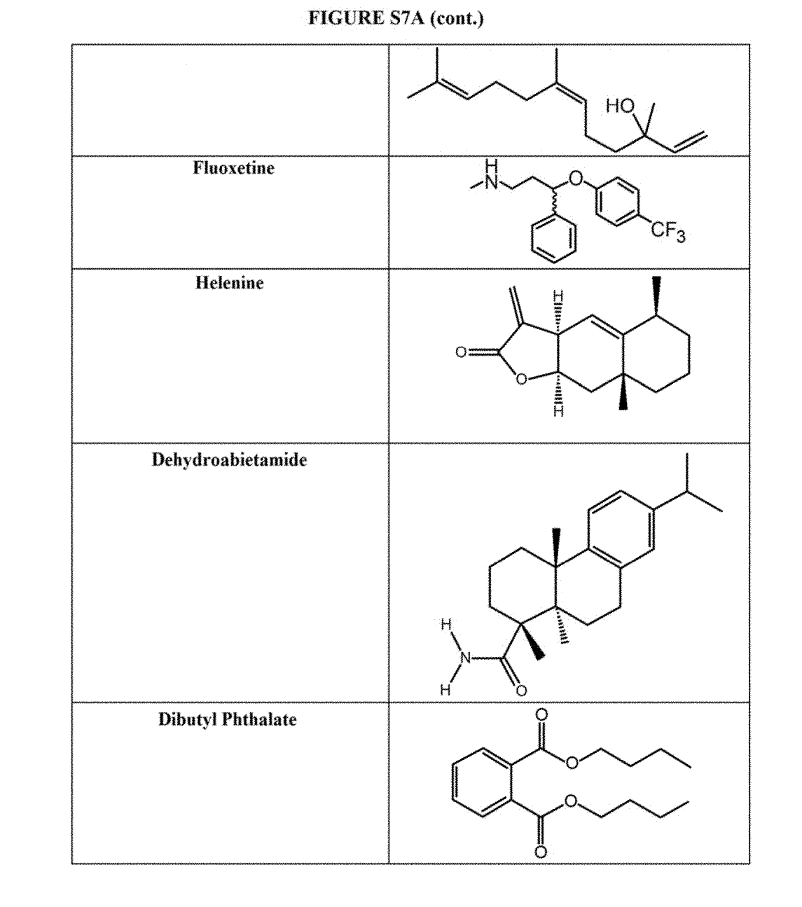

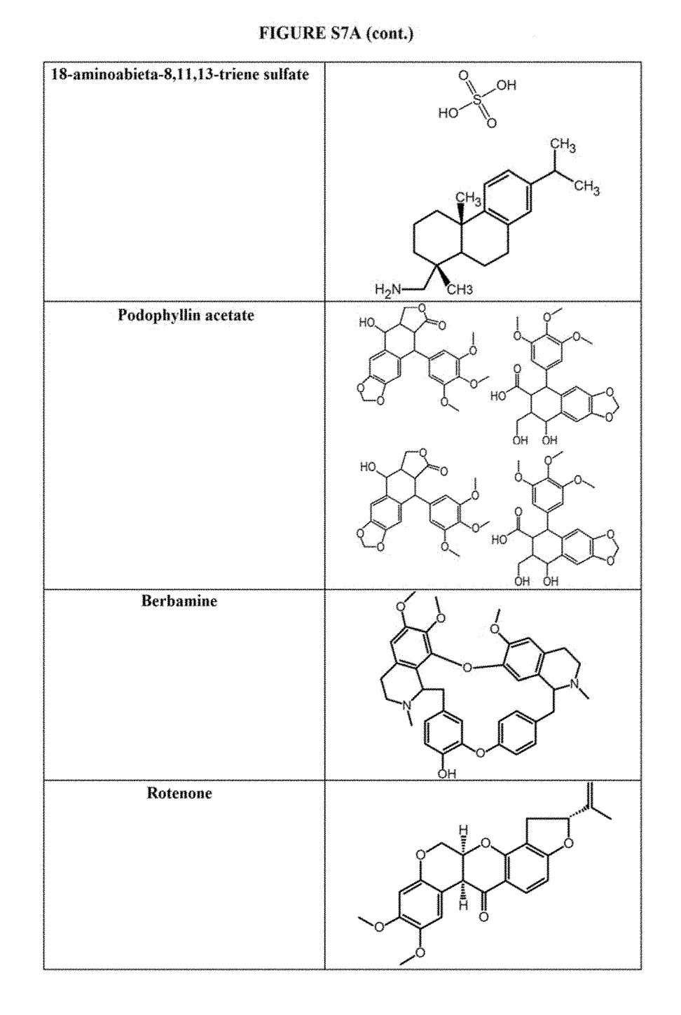

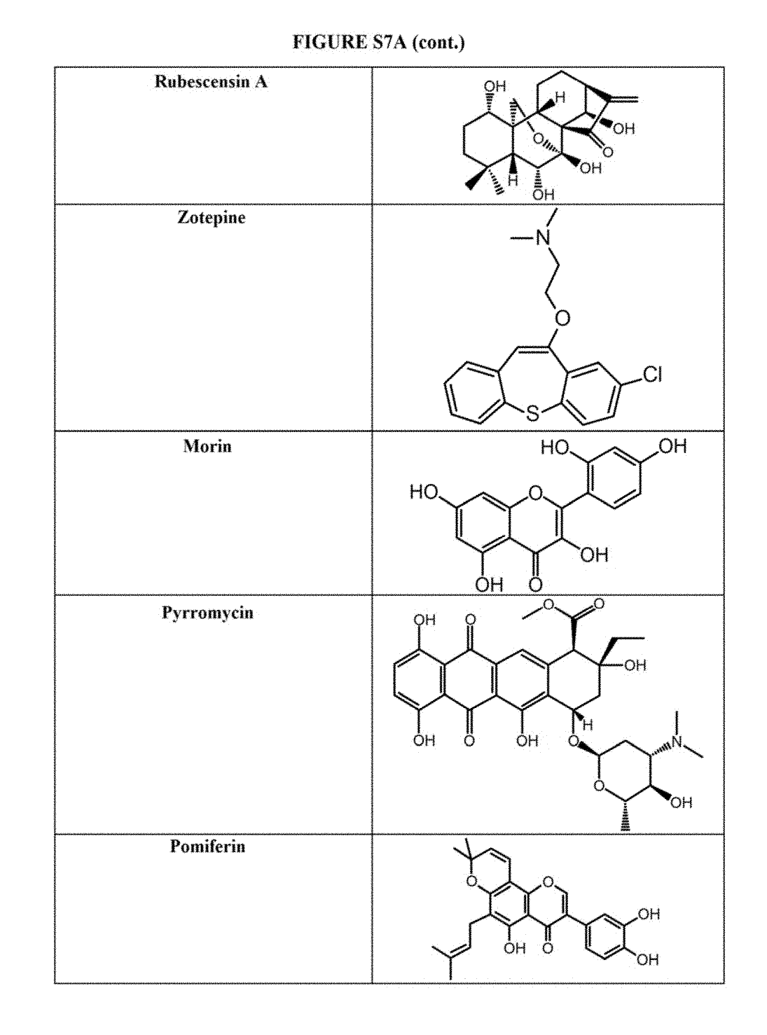

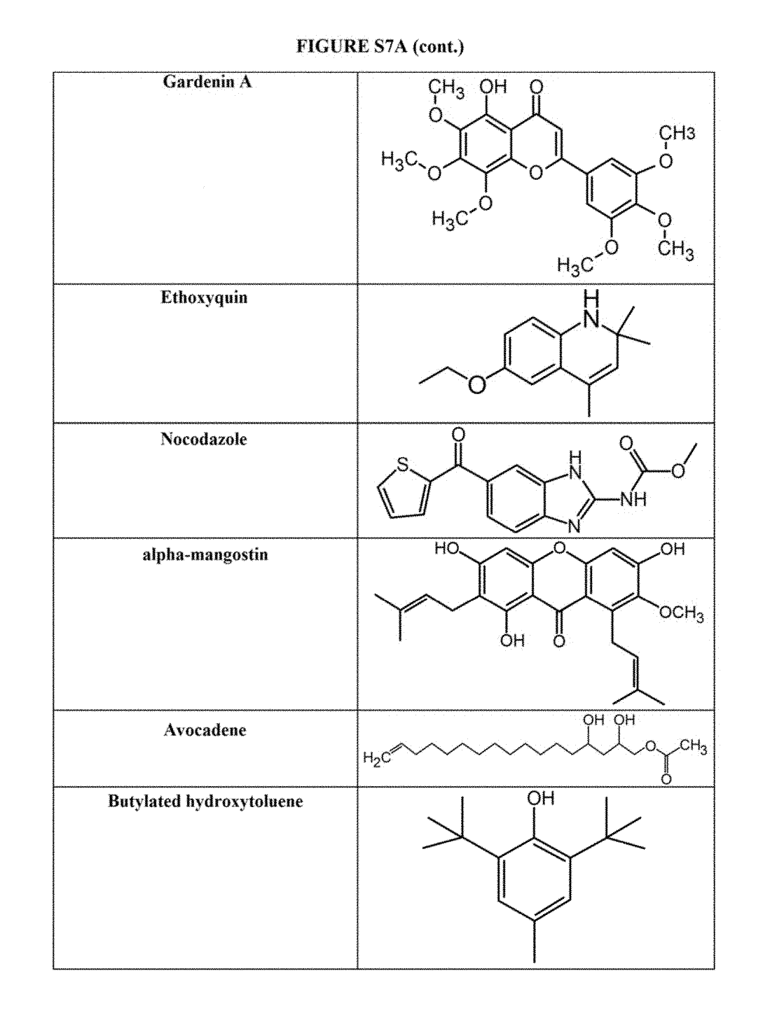

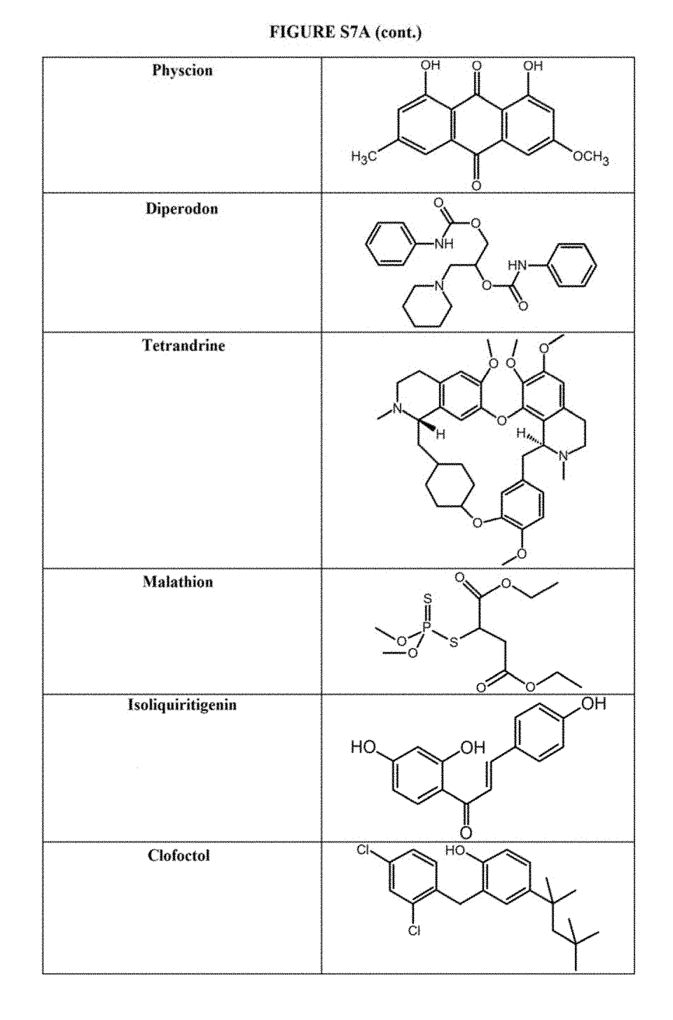

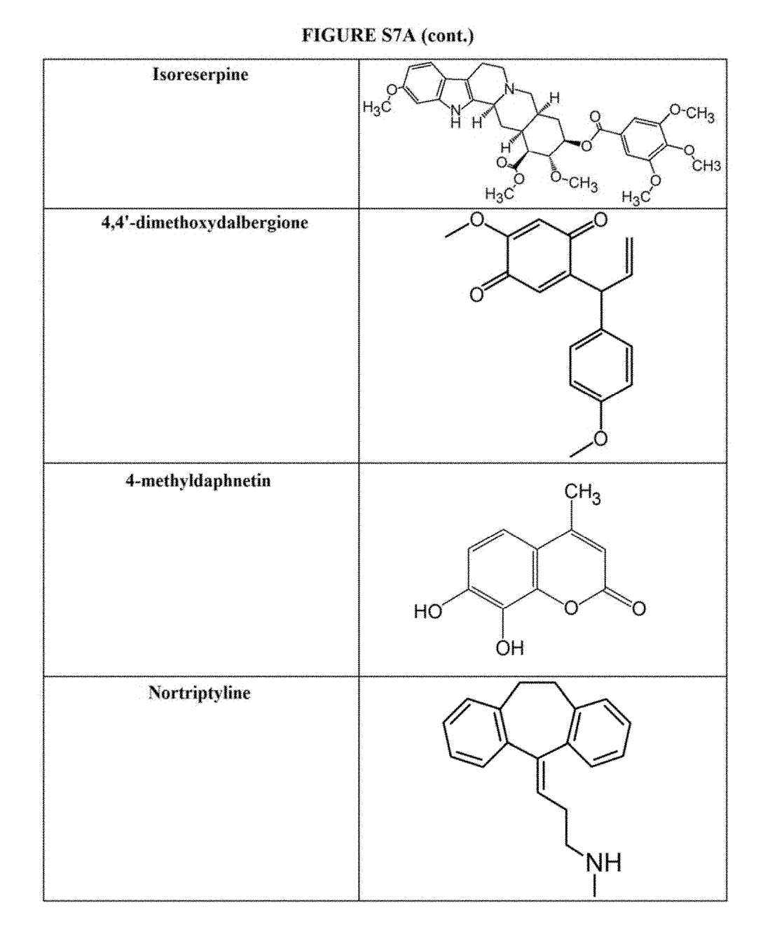

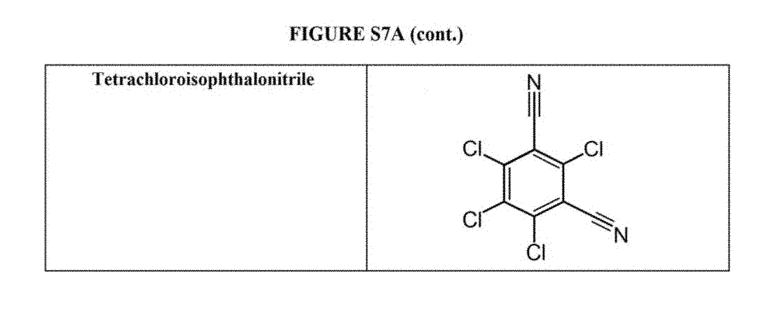

FIG. 7A illustrates that autophagy degrades protein target of TRIM5α in a manner requiring direct target-TRIM5α binding, as determined in the experiment(s) of Example 3.

Figure S1A. TRIM proteins regulate autophagy. (A) Representative images of cells expressing green-fluorescent LC3B transfected with non-targeting siRNA (sScr), siRNA against Beclin 1, or against selected TRIMs (see FIG. 1B) after treatment with pp242. Green, GFP-LC3B. Blue, nuclei. (B) High content image analysis of TRIM siRNA screen as in FIG. 1, plotted here as number of LC3 puncta per cell (all symbols and statistics as in FIG. 1A/A-B); results as determined in the experiment(s) of Example 3.

Figure S2A. TRIM5α interacts with p62/sequestosome 1. (A) Assessment of TRIM5α interaction with GFP or GFP-p62 by co-immunoprecipitation. (B) Confocal immunofluorescence microscopy of cells expressing p62-GFP (green) and HA-tagged TRIM5α (rhesus), blue. Results as determined in the experiment(s) of Example 3.

Figure S3A. TRIM5α interacts with key autophagy factor Beclin 1. (A) Assessment of interaction between endogenous Beclin 1 and endogenous TRIM5α in FRhK4 cells by co-inmmunoprecipitation. (B-C) Proximity ligation analysis (PLA) of in situ interactions between RhTRIM5α and Beclin 1, p62, TAB2, or TAK1 in HeLa cells. A positive direct protein-protein interaction is revealed by a fluorescent dot (images: blue, nuclear stain; red, PLA signal). Schematic: a red dot is a products of in situ PCR that generates a fluorescent (circular) product with primers physically linked to antibodies 1 and 2 (Ab#1, Ab#2). The fluorescent dot appears only if Ab#1 and Ab#2 are separated by less than 16 nm (equivalent to FRET distances between proteins). Quantification (red dots), pairs of Ab#1 and Ab#2 as indicated under the graph. (D) Confocal immunofluorescence microscopy using the antibody pairs and cells as in (B) employed as a control that RhTRIM5α and TAK1 are recognized by Ab#1 and Ab#2 in HeLa cells. (E) Mapping of Beclin 1 regions interacting with GFP-RhTRIM5α (see schematic in FIG. 3E). 293T cells were transfected with the corresponding constructs (Rhesus TRIM5α fused to GFP; Beclin 1 domains tagged with FLAG epitope; 1-450, full size Beclin 1). Lysates were immunoprecipitated with anti-FLAG antisera and immunoblots probed as indicated. Results as determined in the experiment(s) of Example 3.

Figure S4A. TRIM5α is associated with membranes and co-localizes with punctuate LC3 (A) Colocalization analysis of HA-RhTRIM5α and autophagic factors under basal and rapamycin-induced autophagy conditions in HeLa cells. Arrows, overlaps between LC3B and HA-RhTRIM5α. Quantitation, Pearson's colocalization coefficient for p24 CA and indicated markers. (B) Membranous organelles from untreated (top) or rapamycin-treated (bottom) HeLa cells expressing HA-RhTRIM5α were separated by isopycnic centrifugation in sucrose gradients. Arrow indicates decrease in fraction number upon rapamycin treatment. Data, means±SE *, n≥3 experiments, P<0.05 (t test). Results as determined in the experiment(s) of Example 3.

Figure S5A. Autophagy protects rhesus cells from infection with pseudotyped virus containing HIV-1 p24. (A-B) Immunoblot based assessment of HIV-1 p24 in primary rhesus CD4+ T cells subjected to indicated knockdowns, infected with VSVG-pseudotyped HIV-1, and induced for autophagy by starvation for 4 hours. (C) HIV-1 proviral DNA in FRhK4 cells subjected to TRIM5α (RhT5α) or Beclin 1 (Bec) knockdowns and infected with VSVG-pseudotyped HIV-1 for 4 hours. (D) HIV-1 reverse transcriptase (RT) activity in fed or starved rhesus cells (FRhK4) knocked down for indicated factors and infected for 4 hours with VSVG-pseudotyped HIV-1. Data, means±SE; n≥3 experiments.*, P<0.05; †, P≥0.05 (t test). Results as determined in the experiment(s) of Example 3.

Figure S6A. ALFY co-localizes with TRIM5α and is required for optimal degradation of p24. (A) Co-localization analysis (graph, Pearson's colocalization coefficient) for ALFY and HA-RhTRIM5α in HeLa cells. RAP, autophagy induced with rapamycin. CTRL, control (vehicle). Arrows, examples of colocalization between ALFY and HA-RhTRIM5α. (B-C) Effects of rhesus ALFY knockdown on p24 levels in FRhK4 cells following exposure to pseudotyped HIV-1. Cells were incubated in full or starvation media following infection. Data, means±SE; *, P<0.05; †, P≥0.05 (Student's t test; n≥3). Results as determined in the experiment(s) of Example 3.

Figure S7A lists representative autophagy modulators that are useful in compositions and methods of the invention.

### DETAILED DESCRIPTION OF THE INVENTION

It is noted that, as used in this specification and the appended claims, the singular forms “a,” “an,” and “the,” include plural referents unless expressly and unequivocally limited to one referent. Thus, for example, reference to “a compound” includes two or more different compound. As used herein, the term “include” and its grammatical variants are intended to be non-limiting, such that recitation of items in a list is not to the exclusion of other like items that can be substituted or other items that can be added to the listed items.

The term “compound” or “agent”, as used herein, unless otherwise indicated, refers to any specific chemical compound disclosed herein, especially including a neutral lipid (as described herein), a TRIM protein or other autophagy modulator and includes tautomers, regioisomers, geometric isomers as applicable, and also where applicable, optical isomers (e.g. enantiomers) thereof, as well as pharmaceutically acceptable salts thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds as well as diastereomers and epimers, where applicable in context. The term also refers, in context to prodrug forms of compounds which have been modified to facilitate the administration and delivery of compounds to a site of activity.

The term “patient” or “subject” is used throughout the specification within context to describe an animal, generally a mammal, including a domesticated mammal including a farm animal (dog, cat, horse, cow, pig, sheep, goat, etc.) and preferably a human, to whom treatment, including prophylactic treatment (prophylaxis), with the methods and compositions according to the present invention is provided. For treatment of those conditions or disease states which are specific for a specific animal such as a human patient, the term patient refers to that specific animal, often a human.

The terms “effective” or “pharmaceutically effective” are used herein, unless otherwise indicated, to describe an amount of a compound or composition which, in context, is used to produce or affect an intended result, usually the modulation of autophagy within the context of a particular treatment or alternatively, the effect of a neutral lipid and/or TRIM protein which is coadministered with another autophagy modulator and/or another bioactive agent in the treatment of disease.

The terms “treat”, “treating”, and “treatment”, etc., as used herein, refer to any action providing a benefit to a patient at risk for or afflicted by an autophagy mediated disease state or condition as otherwise described herein. The benefit may be in curing the disease state or condition, inhibition its progression, or ameliorating, lessening or suppressing one or more symptom of an autophagy mediated disease state or condition. Treatment, as used herein, encompasses both prophylactic and therapeutic treatment.

The term “neutral lipids” refers to lipids which do not contain a charge. Neutral lipids for use in the present invention include, for example, neutral lipids which are selected from the group consisting of triglycerides, diglycerides, monoglycerides, glycolated mono- or diacylglycerdies, dolichol, polyprenol, polyprenal or very long chain fatty acids, which are uncharged or weakly charged. Neutral lipids for use in the present invention include those that are effective for enhancing lipid stores and promoting lipid droplets such that enhancement of autophagy occurs. Neutral lipids may be administered to a patient in need for the intended effect of enhancing autophagy.

As used herein, the term “autophagy mediated disease state or condition” or an “autophagy-related disorder” refers to a disease state or condition that results from disruption in autophagy or cellular self-digestion. Autophagy is a cellular pathway involved in protein and organelle degradation, and has a large number of connections to human disease. Autophagic dysfunction is associated with cancer, neurodegeneration, microbial infection and ageing, among numerous other disease states and/or conditions. Although autophagy plays a principal role as a protective process for the cell, it also plays a role in cell death. Disease states and/or conditions which are mediated through autophagy (which refers to the fact that the disease state or condition may manifest itself as a function of the increase or decrease in autophagy in the patient or subject to be treated and treatment requires administration of an inhibitor or agonist of autophagy in the patient or subject) include, for example, cancer, including metastasis of cancer, lysosomal storage diseases (discussed hereinbelow), neurodegeneration (including, for example, Alzheimer's disease, Parkinson's disease, Huntington's disease; other ataxias), immune response (T cell maturation, B cell and T cell homeostasis, counters damaging inflammation) and chronic inflammatory diseases (may promote excessive cytokines when autophagy is defective), including, for example, inflammatory bowel disease, including Crohn's disease, rheumatoid arthritis, lupus, multiple sclerosis, chronic obstructive pulmony disease/COPD, pulmonary fibrosis, cystic fibrosis, Sjogren's disease; hyperglycemic disorders, diabetes (I and II), affecting lipid metabolism islet function and/or structure, excessive autophagy may lead to pancreatic β-cell death and related hyperglycemic disorders, including severe insulin resistance, hyperinsulinernia, insulin-resistant diabetes (e.g. Mendenhall's Syndrome, Werner Syndrome, leprechaunism, and lipoatrophic diabetes) and dyslipidemia (e.g. hyperlipidemia as expressed by obese subjects, elevated low-density lipoprotein (LDL), depressed high-density lipoprotein (HDL), and elevated triglycerides) and metabolic syndrome, liver disease (excessive autophagic removal of cellular entities-endoplasmic reticulum), renal disease (apoptosis in plaques, glomerular disease), cardiovascular disease (especially including ischemia, stroke, pressure overload and complications during reperfusion), muscle degeneration and atrophy, symptoms of aging (including amelioration or the delay in onset or severity or frequency of aging-related symptoms and chronic conditions including muscle atrophy, frailty, metabolic disorders, low grade inflammation, atherosclerosis and associated conditions such as cardiac and neurological both central and peripheral manifestations including stroke, age-associated dementia and sporadic form of Alzheimer's disease, pro-cancerous states, and psychiatric conditions including depression), stroke and spinal cord injury, arteriosclerosis, infectious diseases (microbial infections, removes microbes, provides a protective inflammatory response to microbial products, limits adapation of autophagy of host by microbe for enhancement of microbial growth, regulation of innate immunity) including bacterial, fungal, cellular and viral (including secondary disease states or conditions associated with infectious diseases), including AIDS and tuberculosis, among others, development (including erythrocyte differentiation), embryogenesis/fertility/infertility (embryo implantation and neonate survival after termination of transplacental supply of nutrients, removal of dead cells during programmed cell death) and ageing (increased autophagy leads to the removal of damaged organelles or aggregated macromolecules to increase health and prolong lire, but increased levels of autophagy in children/young adults may lead to muscle and organ wasting resulting in aging/progeria).

One preferred category of autophagy-related disorders which may be treated by methods and compositions of the invention includes cancer, including metastasis of cancer, lysosomal storage diseases (discussed in detail hereinbelow), neurodegeneration (including, for example, Alzheimer's disease, Parkinson's disease; other ataxias), immune response, chronic inflammatory diseases, including inflammatory bowel disease, including Crohn's disease, rheumatoid arthritis, lupus, multiple sclerosis, chronic obstructive pulmony disease/COPD, pulmonary fibrosis, cystic fibrosis, Sjogren's disease; diabetes (I and II) and metabolic syndrome, liver disease, renal disease (including glomerular disease), cardiovascular disease (especially including ischemia, stroke, pressure overload and complications during reperfusion), muscle degeneration and atrophy, symptoms of aging (including amelioration or the delay in onset or severity or frequency of aging-related symptoms and chronic conditions including muscle atrophy, frailty, metabolic disorders, low grade inflammation, atherosclerosis and associated conditions such as cardiac and neurological both central and peripheral manifestations including stroke, age-associated dementia and sporadic form of Alzheimer's disease, pre-cancerous states, and psychiatric conditions including depression.), stroke and spinal cord injury, arteriosclerosis, infectious diseases (microbial infections, including bacterial, fungal, cellular, viral (including influenza, herpes virus, HIV, HBV and HCV, among others) and parasitic infections, including protozoal and helminthic, including secondary disease states or conditions associated with infectious diseases), including AIDS and tuberculosis, among others, including in periodontal disease, development, both overly mature and immature development (including erythrocyte differentiation), embryogenesis/fertility and ageing/progeria.

The term “lysosomal storage disorder” refers to a disease state or condition that results from a defect in lysosomomal storage. These disease states or conditions generally occur when the lysosome malfunctions. Lysosomal storage disorders are caused by lysosomal dysfunction usually as a consequence of deficiency of a single enzyme required for the metabolism of lipids, glycoproteins or mucopolysaccharides. The incidence of lysosomal storage disorder (collectively) occurs at an incidence of about about 1:5,000-1:10,000. The lysosome is commonly referred to as the cell's recycling center because it processes unwanted material into substances that the cell can utilize. Lysosomes break down this unwanted matter via high specialized enzymes. Lysosomal disorders generally are triggered when a particular enzyme exists in too small an amount or is missing altogether. When this happens, substances accumulate in the cell. In other words, when the lysosome doesn't function normally, excess products destined for breakdown and recycling are stored in the cell. Lysosomal storage disorders are genetic diseases, but these may be treated using autophagy modulators (autostatins) as described herein. All of these diseases share a common biochemical characteristic, i.e., that all lysosomal disorders originate from an abnormal accumulation of substances inside the lysosome. Lysosomal storage diseases mostly affect children who often die as a consequence at an early stage of life, many within a few months or years of birth. Many other children die of this disease following years of suffering from various symptoms of their particular disorder.

Examples of lysosomal storage diseases include, for example, activator deficiency/GM2 gangliosidosis, alpha-mannosidosis, aspartylglucoaminuria, cholesteryl ester storage disease, chronic hexosaminidase A deficiency, cystinosis, Danon disease, Fabry disease, Farber disease, fucosidosis, galactosialidosis, Gaucher Disease (Types I, II and III), GM1 Ganliosidosis, including infantile, late infantile/juvenile and adult/chronic), Hunter syndrome (MPS II), 1-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurer syndrome, Scheie syndrome, Hurler-Scheie syndrome, Sanfilippo syndrome, Morquio Type A and B, Maroteaux-Lamy, Sly syndrome, muolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs and Wolman disease, among others.

An autophagy-elated disorder may be an “immune disorder”, including, but not limited to, lupus, multiple sclerosis, rheumatoid arthritis, peorlasis, Type I diabetes, complications from organ transplants, xeno transplantation, diabetes, cancer, asthma, atopic dermatitis, autoimmune thyroid disorders, ulcerative colitis, Crohn's disease, Alzheimer's disease and leukemia.

The term “modulator of autophagy”, “regulator of autophagy” or “autostatin” is used to refer to a compound such as a TRIM protein, a neutral lipid as otherwise described herein or other autophagy modulator as otherwise described herein which functions as an agonist (inducer or up-regulator) or antagonist (inhibitor or down-regulator) of autophagy. Depending upon the disease state or condition, autophagy may be upregulated (and require inhibition of autophagy for therapeutic intervention) or down-regulated (and require upregulation of autophagy for therapeutic intervention). In most instances, in the case of cancer treatment with a modulator of autophagy as otherwise described herein, the autophagy modulator is often an antagonist of autophagy. In the case of cancer, the antagonist (inhibitor) of autophagy may be used alone or combined with an agonist of autophagy

The following compounds have been identified as autophagy modulators according to the present invention and can be used in the treatment of an autophagy mediated disease state or condition as otherwise described herein. It is noted that an inhibitor of autophagy is utilized where the disease state or condition is mediated through upregulation or an increase in autophagy which causes the disease state or condition and an agonist of autophagy is utilized where the disease state or condition is mediated through downregulation or a decrease in autophagy. These include the TRIM proteins TRIM5α, TRIM1, TRIM6, TRIM10, TRIM17, TRIM22, TRIM41, TRIM55, TRIM72 and TRIM76, among others including TRIM2, TRIM23, TRIM26, TRIM28, TRIM31, TRIM 32, TRIM33, TRIM38, TRIM42, TRIM44, TRIM45, TRIM49, TRIM50, TRIM51, TRIM58, TRIM59, TRIM65, TRIM68, TRIM73, TRIM74 and TRIM76, among others preferably TRIM 5α, TRIM1, TRIM6, TRIM10, TRIM17, TRIM22, TRIM41, TRIM55, TRIM 72, TRIM76 and mixtures thereof, including pharmaceutically acceptable salts thereof, among others.

The following compounds have also been identified as autophagy modulators (autotaxins) and may also be used in combination with neutral lipids and/or TRIM proteins to treat autophagy-associated disease states and or conditions: flubendazole, hexachlorophene, propidium iodide, bepridil, clomiphene citrate (Z,E), GBR 12909, propafenone, metixene, dipivefrin, fluvoxamine, dicyclomine, dimethisoquin, ticlopidine, memantine, bromhexine, norcyclobenzaprine, diperodon and nortriptyline, tetrachlorisophthalonitrile, phenylmercuric acetate and pharmaceutically acceptable salts thereof. It is noted that flubendazole, hexachlorophene, propidium iodide, bepridil, clomiphene citrate (Z,E), GBR 12909, propafenone, metixene, dipivefrin, fluvoxamine, dicyclomine, dimethisoquin, ticlopidine, memantine, bromhexine, norcyclobenzaprine, diperodon, nortriptyline and their pharmaceutically acceptable salts show activity as agonists or inducers of autophagy in the treatment of an autophagy-mediated disease, whereas tetrachlorisophthalonitrile, phenylmercuric acetate and their pharmaceutically acceptable salts, find use as antagonists or inhibitors of autophagy. All of these compounds will find use as modulators of autophagy in the various autophagy-mediated disease states and conditions described herein, with the agonists being preferred in most disease states other than cancer (although inhibitors may also be used alone, or preferably in combination with the agonists) and in the case of the treatment of cancer, the inhibitors described above are preferred, alone or in combination with an autophagy agonist as described above and/or an additional anticancer agent as otherwise described herein.

Autophagy modulators also include Astemizole, Chrysophanol, Emetine, Chlorosalicylanilide, Oxiconazole, Sibutramine, Proadifen, Dibydroergotamine tartrate, Terfenadine, Triflupromazine, Amiodarone, Saponin Vinblastine, Tannic acid, Fenticlor, Pizotyline malate, Piperacetazine, Oxyphencyclimine, Glyburide, Hydroxychloroquine, Methotrimeprazine, Mepartricin, Thiamylal Sodium Triclocarban, Diphenidol, Karanjin, Clovanediol diacetate, Nerolidol, Fluoxetine, Helenine, Dehydroabietamide, Dibutyl Phthalate, 18-aminoabieta-8,11,13-triene sulfate, Podophyllin acetate, Berbamine, Rotenone, Rubescensin A, Morin, Pyrromycin, Pomiferin, Gardenin A, alpha-mangostin, Avocadene, Butylated hydroxytoluene, Physcion, Tetrandrine, Malathion, Isoliquiritigenin, Clofoctol, Isoreserpine, 4, 4′-dimethoxydalbergione and 4-methyldaphnetin, and mixtures thereof.

Autophagy modulators also include the compounds listed in Figure S7A.

Other compounds which may be used in combination with the neutral lipids, or optionally, the TRIM proteins and/or the above-described autophagy modulators, include for example, other “additional autophagy modulators” or “additional autostatins” which are known in the art. These can be combined with one or more of the autophagy modulators which are disclosed above to provide novel pharmaceutical compositions and/or methods of treating autophagy mediated disease states and conditions which are otherwise described herein. These additional autophagy modulators including benzethonium, niclosamide, monensin, bromperidol, levobunolol, dehydroisoandosterone 3-acetate, sertraline, tamoxifen, reserpine; hexachlorophene, dipyridamole, harmaline, prazosin, lidoflazine, thiethylperazine, dextromethorphan, desipramine, mebendazole, canrenone, chlorprothixene, maprotiline, homochlorcyclizine, loperamide, nicardipine, dexfenfluramine, nilvadipine, dosulepin, biperiden, denatonium, etomidate, toremifene, tomoxetine, clorgyline, zotepine, beta-escin, tridihexethyl, ceftazidime, methoxy-6-harmalan, melengestrol, albendazole, rimantadine, chlorpromazine, pergolide, cloperastine, prednicarbate, haloperidol, clotrimazole, nitrofural, iopanoic acid, naftopidil, Methimazole, Trimeprazine, Ethoxyquin, Clocortolone, Doxycycline, Pirlindole mesylate, Doxazosin, Deptropine, Nocodazole, Scopolamine, Oxybenzone, Halcinonide, Oxybutynin, Miconazole, Clomipramine, Cyproheptadine, Doxepin. Dyclonine, Salbutamol, Flavoxate, Amoxapine, Fenofibrate, Pimethixene and mixtures thereof.

The term “co-administration” or “combination therapy” is used to describe a therapy in which at least two active compounds in effective amounts are used to treat an autophagy mediated disease state or condition as otherwise described herein, either at the same time or within dosing or administration schedules defined further herein or ascertainable by those of ordinary skill in the art. Although the term co-administration preferably includes the administration of two active compounds to the patient at the same time, it is not necessary that the compounds be administered to the patient at the same time, although effective amounts of the individual compounds will be present in the patient at the same time. In addition, in certain embodiments, co-administration will refer to the fact that two compounds are administered at significantly different times, but the effects of the two compounds are present at the same time. Thus, the term co-administration includes an administration in which a neutral lipid and/or a TRIM protein is coadministered with at least one additional active agent (including another autophagy modulator) is administered at approximately the same time (contemporaneously), or from about one to several minutes to about 24 hours or more than the other bioactive agent coadministered with the autophagy modulator. The additional bioactive agent may be any bioactive agent, but is generally selected from an additional autophagy mediated compound, an additional anticancer agent, or another agent, such as a mTOR inhibitor such as pp242, rapamycin, envirolimus, everolimus or cidaforollimus, among others including epigallocatechin gallate (EGCG), caffeine, curcumin or reseveratrol (which mTOR inhibitors find particular use as enhancers of autophagy using the compounds disclosed herein and in addition, in the treatment of cancer with an autophagy modulator (inhibitor) as described herein, including in combination with tetrachlorisophthalonitrile, phenylmercuric acetate and their pharmaceutically acceptable salts, which are inhibitors of autophagy. It is noted that in the case of the treatment of cancer, the use of an autophagy inhibitor is preferred, alone or in combination with an autophagy inducer (agonist) as otherwise described herein and/or a mTOR inhibitor as described above. In certain embodiments, an mTOR inhibitor selected from the group consisting of pp242, rapamycin, envirolimus, everolimus, cidaforollimus, epigallocatechin gallate (EGCG), caffeine, curcumin, reseveratrol and mixtures thereof may be combined with at least one agent selected from the group consisting of digoxin, xylazine, hexetidine and sertindole, the combination of such agents being effective as autophagy modulators in combination.

The term “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated.

As used herein, the term neoplasia is used to describe all cancerous disease states and embraces or encompasses the pathological process associated with malignant hematogenous, ascitic and solid tumors. Representative cancers include, for example, stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma, non-melanoma skin cancer (especially basal cell carcinoma or squamous cell carcinoma), acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention. In certain aspects, the cancer which is treated is lung cancer, breast cancer, ovarian cancer and/or prostate cancer.

The term “tumor” is used to describe a malignant or benign growth or tumefacent.

The term “additional anti-cancer compound”, “additional anti-cancer drug” or “additional anti-cancer agent” is used to describe any compound (including its derivatives) which may be used to treat cancer. The “additional anti-cancer compound”, “additional anti-cancer drug” or “additional anti-cancer agent” can be an anticancer agent which is distinguishable from a CIAE-inducing anticancer ingredient such as a taxane, vinca alkaloid and/or radiation sensitizing agent otherwise used as chemotherapy/cancer therapy agents herein. In many instances, the co-administration of another anti-cancer compound according to the present invention results in a synergistic anti-cancer effect. Exemplary anti-cancer compounds for co-administration with formulations according to the present invention include anti-metabolites agents which are broadly characterized as antimetabolites, inhibitors of topoisomerase I and II, alkylating agents and microtubule inhibitors (e.g., taxol), as well as tyrosine kinase inhibitors (e.g., surafenib), EGF kinase inhibitors (e.g., tarceva or erlotinib) and tyrosine kinase inhibitors or ABL kinase inhibitors (e.g. imatinib).

Anti-cancer compounds for co-administration include, for example, Aldesleukin; Alemtuzumab; alitretinoin; allopurinol; altretamine amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; gleevec (imatinib); goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; pocarbazine; quinacrine, Rasburicase; Rituximab; Sargramostim; streptozocin; surafenib; talbuvidine (LDT); talc; tamoxifen; tarceva (erlotinib); temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and mixtures thereof, among others.

Co-administration of one of the formulations of the invention with another anticancer agent will often result in a synergistic enhancement of the anticancer activity of the other anticancer agent, an unexpected result. One or more of the present formulations comprising a neutral lipid and/or a TRIM protein, optionally further including another autophagy modulator as described herein (e.g., an autostatin) may also be co-administered with another bioactive agent (e.g., antiviral agent, antihyperproliferative disease agent, agents which treat chronic inflammatory disease, among others as otherwise described herein).

The term “antiviral agent” refers to an agent which may be used in combination with autophagy modulators (autostatins) as otherwise described herein to treat viral infections, especially including HIV infections, HBV infections and/or HCV infections. Exemplary anti-HIV agents include, for example, nucleoside reverse transcriptase inhibitors (NRTI), non-nucleoside reverse transcriptase inhibitors (NNRTI), protease inhibitors, fusion inhibitors, among others, exemplary compounds of which may include, for example, 3TC (Lamivudine), AZT (Zidovudine), (−)-FTC, ddI (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof, including anti-HIV compounds presently in clinical trials or in development. Exemplary anti-HBV agents include, for example, hepsera (adefovir dipivoxil), lamivudine, entecavir, telbivudine, tenofovir, emtricitabine, clevudine, valtoricitabine, amdoxovir, pradefovir, racivir, BAM 205, nitazoxanide, UT 231-B, Bay 41-4109, EHT899, zadaxin (thymosin alpha-1) and mixtures thereof. Anti-HCV agents include, for example, interferon, pegylated intergeron, ribavirin, NM 283, VX-950 (telaprevir), SCH 50304, TMC435, VX-500, BX-813, SCH503034, R1626, ITMN-191 (R7227), R7128, PF-868554, TT033, CGH-759, GI 5005. MK-7009, SIRNA-034, MK-0608, A-837093, GS 9190, ACH-1095, GSK625433, TG4040 (MVA-HCV), A-831, F351, NS5A, NS4B, ANA598, A-689, GNI-104, IDX102, ADX184, GL59728, GL60667, PSI-7851, TLR9 Agonist, PHX1766, SP-30 and mixtures thereof.

According to various embodiments, the compounds according to the present invention may be used for treatment or prevention purposes in the form of a pharmaceutical composition. This pharmaceutical composition may comprise one or more of an active ingredient as described herein.

As indicated, the pharmaceutical composition may also comprise a pharmaceutically acceptable excipient, additive or inert carrier. The pharmaceutically acceptable excipient, additive or inert carrier may be in a form chosen from a solid, semi-solid, and liquid. The pharmaceutically acceptable excipient or additive may be chosen from a starch, crystalline cellulose, sodium starch glycolate, polyvinylpyrolidone, polyvinylpolypyrolidone, sodium acetate, magnesium stearate, sodium laurylsulfate, sucrose, gelatin, silicic acid, polyethylene glycol, water, alcohol, propylene glycol, vegetable oil, corn oil, peanut oil, olive oil, surfactants, lubricants, disintegrating agents, preservative agents, flavoring agents, pigments, and other conventional additives. The pharmaceutical composition may be formulated by admixing the active with a pharmaceutically acceptable excipient or additive.

The pharmaceutical composition may be in a form chosen from sterile isotonic aqueous solutions, pills, drops, pastes, cream, spray (including aerosols), capsules, tablets, sugar coating tablets, granules, suppositories, liquid, lotion, suspension, emulsion, ointment, gel, and the like. Administration route may be chosen from subcutaneous, intravenous, intestinal, parenteral, oral, buccal, nasal, intramuscular, transcutaneous, transdermal, intranasal, intraperitoneal, and topical. The pharmaceutical compositions may be immediate release, sustained/controlled release, or a combination of immediate release and sustained/controlled release depending upon the compound(s) to be delivered, the compound(s), if any, to be co-administered, as well as the disease state and/or condition to be treated with the pharmaceutical composition. A pharmaceutical composition may be formulated with differing compartments or layers in order to facilitate effective administration of any variety consistent with good pharmaceutical practice.

The subject or patient may be chosen from, for example, a human, a mammal such as domesticated animal (e.g., cat, dog, cow, horse, goat, sheep, or a related domesticated and/or farm animal), or other animal. The subject may have one or more of the disease states, conditions or symptoms associated with autophagy as otherwise described herein.

The compounds according to the present invention may be administered in an effective amount to treat or reduce the likelihood of an autophagy-mediated disease and/or condition as well one or more symptoms associated with the disease state or condition. One of ordinary skill in the art would be readily able to determine an effective amount of active ingredient by taking into consideration several variables including, but not limited to, the animal subject, age, sex, weight, site of the disease state or condition in the patient, previous medical history, other medications, etc.

For example, the dose of an active ingredient which is useful in the treatment of an autophagy mediated disease state, condition and/or symptom for a human patient is that which is an effective amount and may range from as little as 100 μg or even less to at least about 500 mg or more, especially several to hundreds of grams or more of a neutral lipid, which may be administered in a manner consistent with the delivery of the drug and the disease state or condition to be treated. In the case of oral administration, active is generally administered from one to four times or more daily. Transdermal patches or other topical administration may administer drugs continuously, one or more times a day or less frequently than daily, depending upon the absorptivity of the active and delivery to the patient's skin. Of course, in certain instances where parenteral administration represents a favorable treatment option, intramuscular administration or slow IV drip may be used to administer active. The amount of active ingredient which is administered to a human patient preferably ranges from about 0.05 mg/kg to about 100 mg/kg or more, about 0.05 mg/kg to about 10 mg/kg, about 0.1 mg/kg to about 7.5 mg/kg, about 0.25 mg/kg to about 6 mg/kg., about 1.25 to about 5.7 mg/kg.

The dose of a compound according to the present invention may be administered at the first signs of the onset of an autophagy mediated disease state, condition or symptom. For example, the dose may be administered for the purpose of lung or heart function and/or treating or reducing the likelihood of any one or more of the disease states or conditions which become manifest during an inflammation-associated metabolic disorder or tuberculosis or associated disease states or conditions, including pain, high blood pressure, renal failure, or lung failure. The dose of active ingredient may be administered at the first sign of relevant symptoms prior to diagnosis, but in anticipation of the disease or disorder or in anticipation of decreased bodily function or any one or more of the other symptoms or secondary disease states or conditions associated with an autophagy mediated disorder to condition.

A “biomarker” is any gene or protein whose level of expression in a biological sample is altered compared to that of a pro-determined level. The pre-determined level can be a level found in a biological sample from a normal or healthy subject. Biomarkers include genes and proteins, and variants and fragments thereof. Such biomarkers include DNA comprising the entire or partial sequence of the nucleic acid sequence encoding the biomarker, or the complement of such a sequence. The biomarker nucleic acids also include RNA comprising the entire or partial sequence of any of the nucleic acid sequences of interest. A biomarker protein is a protein encoded by or corresponding to a DNA biomarker of the invention. A biomarker protein comprises the entire or partial amino acid sequence of any of the biomarker proteins or polypeptides. Biomarkers can be detected, e.g. by nucleic acid hybridization, antibody binding, activity assays, polymerase chain reaction (PCR), S1 nuclease assay and gene chip.

A “control” as used herein may be a positive or negative control as known in the art and can refer to a control cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. For instance, as can be appreciated by a skilled artisan, a control may comprise data from one or more control subjects that is stored in a reference database. The control may be a subject who is similar to the test subject (for instance, may be of the same gender, same race, same general age and/or same general health) but who is known to not have a fibrotic disease. As can be appreciated by a skilled artisan, the methods of the invention can also be modified to compare a test subject to a control subject who is similar to the test subject (for instance, may be of the same gender, same race, same general age and/or same general health) but who is known to express symptoms of a disease. In this embodiment, a diagnosis of a disease or staging of a disease can be made by determining whether protein or gene expression levels as described herein are statistically similar between the test and control subjects.

The terms “level” and/or “activity” as used herein further refer to gene and protein expression levels or gene or protein activity. For example, gene expression can be defined as the utilization of the information contained in a gene by transcription and translation leading to the production of a gene product.

In certain non-limiting embodiments, an increase or a decrease in a subject or test sample of the level of measured biomarkers (e.g. proteins or gene expression) as compared to a comparable level of measured proteins or gene expression in a control subject or sample can be an increase or decrease in the magnitude of approximately ±5,000-10,000%, or approximately ±2,500-5,000%, or approximately ±1,000-2,500%, or approximately ±500-1,000%, or approximately ±250-500%, or approximately ±100-250%, or approximately ±50-100%, or approximately ±25-50%, or approximately ±10-25%, or approximately ±10-20%, or approximately ±10-15%, or approximately ±5-10%, or approximately ±1-5%, or approximately ±0.5-1%, or approximately ±0.1-0.5%, or approximately ±0.01-0.1%, or approximately ±0.001-0.01%, or approximately ±0.0001-0.001%.

The values obtained from controls are reference values representing a known health status and the values obtained from test samples or subjects are reference values representing a known disease status. The term “control”, as used herein, can mean a sample of preferably the same source (e.g. blood, serum, tissue etc.) which is obtained from at least one healthy subject to be compared to the sample to be analyzed. In order to receive comparable results the control as well as the sample should be obtained, handled and treated in the same way. In certain examples, the number of healthy individuals used to obtain a control value may be at least one, preferably at least two, more preferably at least five, most preferably at least ten, in particular at least twenty. However, the values may also be obtained from at least one hundred, one thousand or ten thousand individuals.

A level and/or an activity and/or expression of a translation product of a gene and/or of a fragment, or derivative, or variant of said translation product, and/or the level or activity of said translation product, and/or of a fragment, or derivative, or variant thereof, can be detected using an immunoassay, an activity assay, and/or a binding assay. These assays can measure the amount of binding between said protein molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g. ELISAs, Western blots and other techniques known to those of ordinary skill in the art (see Harlow and Lane, Antibodies: A Laboratory Manual, Cold Spring Harbor Laboratory Press, Cold Spring Harbor, N.Y., 1999 and Edwards R, Immunodiagnostics: A Practical Approach, Oxford University Press, Oxford; England, 1999). All these detection techniques may also be employed in the format of microarrays, protein-arrays, antibody microarrays, tissue microarrays, electronic biochip or protein-chip based technologies (see Schena M., Microarray Biochip Technology, Eaton Publishing, Natick, Mass., 2000).

Certain diagnostic and screening methods of the present invention utilize an antibody, preferably, a monoclonal antibody, capable of specifically binding to a protein as described herein or active fragments thereof. The method of utilizing an antibody to measure the levels of protein allows for non-invasive diagnosis of the pathological states of kidney diseases. In a preferred embodiment of the present invention, the antibody is human or is humanized. The preferred antibodies may be used, for example, in standard radioimmunoassays or enzyme-linked immunosorbent assays or other assays which utilize antibodies for measurement of levels of protein in sample. In a particular embodiment, the antibodies of the present invention am used to detect and to measure the levels of protein present in a renal cell or urine sample.

Humanized antibodies are antibodies, or antibody fragments, that have the same binding specificity as a parent antibody. (i.e., typically of mouse origin) and increased human characteristics. Humanized antibodies may be obtained, for example, by chain shuffling or by using phage display technology. For example, a polypeptide comprising a heavy or light chain variable domain of a non-human antibody specific for a disease related protein is combined with a repertoire of human complementary (light or heavy) chain variable domains. Hybrid pairings specific for the antigen of interest are selected. Human chains from the selected pairings may then be combined with a repertoire of human complementary variable domains (heavy or light) and humanized antibody polypeptide dimers can be selected for binding specificity for an antigen. Techniques described for generation of humanized antibodies that can be used in the method of the present invention are disclosed in, for example, U.S. Pat. Nos. 5,565,332; 5,585,089; 5,694,761; and 5,693,762. Furthermore, techniques described for the production of human antibodies in transgenic mice are described in, for example, U.S. Pat. Nos. 5,545,806 and 5,569,825.

In order to identify small molecules and other agents useful in the present methods for treating or preventing a renal disorder by modulating the activity and expression of a disease-related protein and biologically active fragments thereof can be used for screening therapeutic compounds in any of a variety of screening techniques. Fragments employed in such screening tests may be free in solution, affixed to a solid support, borne on a cell surface, or located intracellularly. The blocking or reduction of biological activity or the formation of binding complexes between the disease-related protein and the agent being tested can be measured by methods available in the art.

Other techniques for drug screening which provide for a high throughput screening of compounds having suitable binding affinity to a protein, or to another target polypeptide useful in modulating, regulating, or inhibiting the expression and/or activity of a disease, are known in the art. For example, microarrays carrying test compounds can be prepared, used, and analyzed using methods available in the art. See, e.g., Shalon, D. et al., 1995, International Publication No. WO95/35505, Baldeschweiler et al., 1995, International Publication No. WO95/251116; Brennan et al., 1995, U.S. Pat. No. 5,474,796; Heller et al., 1997, U.S. Pat. No. 5,605,662.

Identifying small molecules that modulate protein activity can also be conducted by various other screening techniques, which can also serve to identify antibodies and other compounds that interact with proteins identified herein and can be used as drugs and therapeutics in the present methods. See, e.g., Enna et al., eds., 1998, Current Protocols in Pharmacology, John Wiley & Sons, Inc., New York N.Y. Assays will typically provide for detectable signals associated with the binding of the compound to a protein or cellular target. Binding can be detected by, for example, fluorophores, enzyme conjugates, and other detectable labels well known in the art. The results may be qualitative or quantitative.

For screening the compounds for specific binding, various immunoassays may be employed for detecting, for example, human or primate antibodies bound to the cells. Thus, one may use labeled anti-hIg, e.g., anti-hIgM, hIgG or combinations thereof to detect specifically bound human antibody. Various labels can be used such as radioisotopes, enzymes, fluorescers, chemiluminescers, particles, etc. There are numerous commercially available kits providing labeled anti-hIg, which may be employed in accordance with the manufacturer's protocol.

In one embodiment, a kit can comprise: (a) at least one reagent which is selected from the group consisting of (i) reagents that detect a transcription product of the gene coding for a protein marker as described herein (ii) reagents that detect a translation product of the gene coding for proteins, and/or reagents that detect a fragment or derivative or variant of said transcription or translation product; (b) optionally, one or more types of cells, including engineered cells in which cellular assays are to be conducted; (c) instructions for diagnosing, or prognosticating a disease, or determining the propensity or predisposition of a subject to develop such a disease or of monitoring the effect of a treatment by determining a level, or an activity, or both said level and said activity, and/or expression of said transcription product and/or said translation product and/or of fragments, derivatives or variants of the foregoing, in a sample obtained from said subject; and comparing said level and/or said activity and/or expression of said transcription product and/or said translation product and/or fragments, derivatives or variants thereof to a reference value representing a known disease status (patient) and/or to a reference value representing a known health status (control) and/or to a reference value; and analyzing whether said level and/or said activity and/or expression is varied compared to a reference value representing a known health status, and/or is similar or equal to a reference value representing a known disease status or a reference value; and diagnosing or prognosticating a disease, or determining the propensity or predisposition of said subject to develop such a disease, wherein a varied or altered level, expression or activity, or both said level and said activity, of said transcription product and/or said translation product and/or said fragments, derivatives or variants thereof compared to a reference value representing a known health status (control) and/or wherein a level, or activity, or both said level and said activity, of said transcription product and/or said translation product and/or said fragments, derivatives or variants thereof is similar or equal to a reference value and/or to a reference value representing a known disease stage, indicates a diagnosis or prognosis of a disease, or an increased propensity or predisposition of developing such a disease, a high risk of developing signs and symptoms of a disease.

Reagents that selectively detect a transcription product and/or a translation product of the gene coding for proteins can be sequences of various length, fragments of sequences, antibodies, aptamers, siRNA, microRNA, and ribozymes. Such reagents may be used also to detect fragments, derivatives or variants thereof. The invention is described further in the following illustrative examples.

### Example 1

### Neutral Lipid Store and Lipase PNPLA5 Contribute to Autophagosome Biogenesis

Here we show that lipid droplets as cellular stores of neutral lipids including

triglycerides contribute to autophagic initiation. Lipid droplets, as previously shown, were consumed upon induction of autophagy by starvation. However, inhibition of autophagic maturation by blocking acidification or using dominant negative Atg4C74A that prohibits autophagosomal closure, did not prevent disappearance of lipid droplets. Thus, lipid droplets continued to be utilized upon induction of autophagy but not as autophagic substrates in a process referred to as lipophagy. We considered an alternative model whereby lipid droplets were consumed not as a part of lipophagy but as a potential contributing source to the biogenesis of lipid precursors for nascent autophagosomes. We carried out a screen for a potential link between triglyceride mobilization and autophagy, and identified a neutral lipase, PNPLA5, as being required for efficient autophagy. PNPLA5, which localized to lipid droplets, was needed for optimal initiation of autophagy. PNPLA5 was required for autophagy of diverse substrates including degradation of autophagic adaptors, bulk proteolysis, mitochondrial quantity control, and microbial clearance.

### CONCLUSIONS

Lipid droplets contribute to autophagic capacity in a process dependent on PNPLA5.

Thus, neutral lipid stores are mobilized during autophagy to support autophagic membrane formation.

### Summary of Experimental Results

FIG. 1 shows that preformed lipid droplets enhance starvation-induced autophagy. Panals (A) and (B): HeLa cells stably expressing mRFP-GFP-LC3 were treated for 20 h with BSA alone (BSA) or with BSA-oleic acid (OA; 500 μM OA) and then starved in EBSS for 90 min (Starv) or incubated in full medium (Full). A, Visualization of lipid droplet accumulation. Cells were treated as in A and lipid droplets stained with Bodipy 493/503. B, High content image analysis. Program (iDev)-assigned masks: blue outline, valid primary objects (cells); green mask, GFP puncta. (C) number of GFP+ puncta per cell from experiments illustrated in A. (D) RFP+ puncta per cell from experiments illustrated in A. High content analysis data: means±s.e. (n=3, where n represents separate experiments; each experimental point in separate experiments contained >500 cells identified by the program as valid primary objects); *, p<0.05 (t-test). (E) HeLa cells were treated with 0, 100, 500 or 1000 μM oleic acid (OA) using BSA as a carrier (BSA-OA) for 20 h, followed by 90 min starvation in EBSS with or without Bafilomycin A1 (BafA1), and LC3-II/actin ratios determined by immunoblotting and densitometry. (F) HeLa cells were treated for 20 h with BSA alone or with BSA-OA (500 μM OA), followed by starvation in EBSS for 90 min with or without Bafilomycin A1 (BafA1) and LC3-II/actin ratios determined by immunoblotting and densitometry. Immunoblotting data: means means±s.e., *, p<0.05 (t-test).

FIG. 2 shows that early autophagic markers colocalize with LDs.

Panals (A-H): Confocal microscopy analysis of U2OS cells stably expressing GFP WIPI-1 (A), GFP WIPI-2B (C) and GFP WIPI-2D (E) or HEK-293 cells stably expressing GFP-DFCP1 (G). Lipid droplets were visualized (blue channel) with LipidTox DeepRed. Cells were pre-treated for 20 h with 500 μM BSA-Oleic Acid, starved for 1 h (for DFCP1) or 2 h (for WIPIs) and then analyzed by confocal fluorescence microscopy. Arrowheads, colocalization of WIPIs or DFCP1 with lipid droplets. (B,D,F,H) Two-fluorescence channel line tracings corresponding to dashed lines in images to the loft. (I,J) Subcellular fractionation of membranous organelles in oleic acid-treated cells subjected to starvation (Starv) or not (Ctrl; control). HeLa cells were treated with 200 μM BSA-Oleic Acid and then incubated in full medium (I) or starved (J) for 2 h. Cells were then subjected to subcellular fractionation of membranous organelles by isopycnic separation in sucrose density gradients via equilibrium centrifugation. PNS, postnuclear supernatant. Rectangle over fraction 1, convergence in light fractions of early autophagic marker (Atg16L1) with lipid droplets (revealed by ADRP, also known as perilipin 2 or adipophilin). Numbers below lanes, refractive index (reflecting sucrose density) of each fraction. (K) Still frames from intravital imaging of intact live liver in GFP-LC3 mice (see Supplementary Movie 1). Red, lipid droplets; green, GFP-LC3. Time, h:min:s. Arrowheads, a green (GFP-LC3 positive) organelle interacting with a lipid droplet (red).

FIG. 3 shows that lipid droplets are consumed during autophagic induction independently of autophagosomal closure and autophagic maturation. Panals (A-C): HeLa cells were treated for 20 h with BSA alone or with 500 μM BSA-Oleic Acid (OA) and starved (Starv) or not (Full) for 2 h with or without Bafilomycin A1 (Baf) to inhibit autophagic degradation. Cells were then fixed and lipid droplets were stained for fluorescence microscopy with Bodipy 493/503. Lipid droplets (LD) number (B) and total LD area (C) (as illustrated in fluorescent images in A) were quantified by high content image acquisition and analysis. (D,E) Stable 3′13 cells expressing Atg4B or mStrawberry-Atg4B^{C74A} were treated for 20 h with 500 μM BSA-Oleic Acid (OA) and starved or not during 2 h. p62/actin ratios were determined by immunoblotting (D) followed by densitometry (E). Immunoblot analysis data, means±s.e. (n≥3); *, p<0.05. (F,G) Stable 3T3 cells expressing Atg4B or mStrawberry-Atg4B^{C74A} were treated 20 hours with BSA alone or with 500 μM BSA-Oleic Acid (OA) and starved or not for 2 h. Cells were then fixed and lipid droplets stained with Bodipy 493/503. LD number (F) and total LD area (G) were determined by high content image acquisition and analysis. All high content analysis data, means±s.e. (n=3, where n represents separate experiments; each experimental point in separate experiments contained >500 cells identified by the program as valid primary objects); *, p<0.05 †, p≥0.05 (t-test).

FIG. 4 shows a screen for triglyceride metabolism factors that identifies PNPLA5, CPT1 and LPCAT2 as positive regulators of autophagy. Panal (A): Schematic representing triglyceride (TG) mobilization (lipolysis; right arrow) to diacylglycerol (DAG) and DAG re-esterification to TG (left arrow). DGAT, diacylglycerol acyl-tranferase; PNPLAs (1, 2, 3, 4 and 5), PNPLAs, papatin-like phospholipase domain-containing proteins 1 through 5. (B-D) HeLa cells stably expressing mRFP-GFP-LC3 were transfected once (for DGATs) or twice (for PNPLAs) with scrambled (Scr) control siRNA or siRNAs against DGAT1, DGAT2, PNPLA1, PNPLA2, PNPLA3, PNPLA4, and PNPLA5. After 48 h (for DGAT) or 24 h (for PNPLA), cells were treated for 20 h with 500 μM BSA-Oleic Acid (OA) and starved or not for 90 min. GFP+ puncta per cell as illustrated in fluorescent images (B) were quantified by high content image acquisition and analysis. (E,F) Effect of PNPLA5 on autophagy induction by measuring LC3-11 levels. HeLa cells were transfected twice with siRNAs (PNPLA 2,3,5) or scrambled (Scr) control. Cells were then treated 20 h with 500 μM BSA-Oleic Acid (OA) and starved for 90 min with or without Bafilomycin A1 (Bat) to inhibit autophagic degradation and LC3-II/actin ratios determined by immunoblotting (E) followed by densitometry (F). Immunoblotting data, means±s.e. (n≥3); *, p<0.05. (G,H) Effect of PNPLA5 overexpression on autophagy induction by quantifying endogenous LC3 dots. HeLa cells were transfected either with GFP or with PNPLA5-GFP expression plasmids, and then treated 20 h with 500 μM BSA-Oleic Acid (OA). After that, cells were starved for 2 h with or without Bafilomycin A1 (Baf). Endogenous LC3 was stained by immunofluorescence and LC3 dots were quantified within GFP positive cells (as illustrated in fluorescent images in G) by high content image acquisition and analysis in H. (I,J) Confocal microscopy of HeLa cells transfected with PNPLA5-GFP expression plasmid (green cell), Atg16L1 (red) and lipid droplets (LD, LipidTox DeepRed, blue channel). Cells were transfected with PNPLA5-GFP expressing plasmid, and treated for 20 h with 500 μM BSA-Oleic Acid (OA). Cells were fixed, lipid droplets stained with LipidTox DeepRed and Atg16L1 stained with antibodies for immunofluorescence microscopy. Arrowhead, colocalization of PNPLA5GFP, Atg16L1, and lipid droplets; dashed line, two-fluorescence channel line tracing shown in panel J. (K) Scheme, enzymes involved in the phospholipid synthesis pathway. (L) HeLa cells stably expressing mRFP-GFP-LC3 were transfected twice with scramble control (Scr) or CPT1 siRNAs. After 24 h, cells were treated for 20 h with 500 μM BSA-Oleic Acid (OA) and starved for 90 min. GFP+ puncta per cell were quantified by high content image acquisition and analysis. (M) Schematic of the Lands cycle phospholipid remodeling pathway; PLA2, phospholipase A2, LPC, lysophosphatidylcholine; LPCAT, lysophosphatidylcholine acyl-transferase (1 and 2), PC, phosphatidylcholine. (N) HeLa cells stably expressing mRFP-GFP-LC3 were transfected with scrambles control (Scr), LPCAT1, or LPCAT2 siRNAs. 48 h following transfection, cells were treated for 20 h with 500 μM BSA-Oleic Acid (OA) and starved or not for 90 min. GFP+ puncta per cell were quantified by high content image acquisition and analysis. All high content analysis data, means±s.e (n=3, where n represents separate experiments; each experimental point in separate experiments contained >500 cells identified by the program as valid primary objects); *, p<0.05 †, p=0.05 (t-test).

FIG. 5 shows co-localization between DAG, Atg16L1 and lipid droplets upon overexpression of mutant Atg4B^{C74A}. Panal (A): Analysis of diacylglicerol (DAG) localization (revealed by the NES-GFP-DAG probe; see Supplementary FIG. 3) on lipid droplets (LD). Starv, autophagy induced by starvation; Full, full medium. (B) Analysis of Atg16L1 localization (revealed by antibody staining) to lipid droplets (LD). (C) Analysis of Atg16L1-DAG colcoalization. Colocalization was quantified by SlideBook morphometric analysis software (see Materials and Methods). Data mean values±s.e. (n≥3); *, p<0.05. (D) Atg16L1 and DAG colocalization (white asterisks). Line tracing, analysis of fluorescence signal intensity from images shown in Supplementary Figure D. Arrowhead, overlap between Atg16L1 and DAG signal.

FIG. 6 shows that PNPLA5 is required for efficient autophagy of diverse autophagic substrates. Panals (A,B): effect of PNPLA5 knockdown on lipid droplets degradation upon autophagy induction. HeLa cells were transfected twice with PNPLA5 or scramble (Scr) siRNA control. Cells were treated for 20 h with BSA or with 500 μM BSA-Oleic Acid (OA) and starved or not during 2 h. Cells were then fixed and lipid droplets stained by immunofluorescence with Bodipy 493/503. LD number (A) and total LD area (B) per cell (illustrated in Supplementary FIG. 4) were determined by high content image acquisition and analysis. Data, means±s.e. (n≥3); *, p<0.05. (C-E) Effect of PNPLA5 on P62 autophagic degradation. HeLa cells were transfected twice with PNPLA5 siRNAs or scramble (Scr) control. Cells were then treated 20 hours with 500 μM BSA-Oleic Acid (OA) and starved or not during 2 hours. (C) Endogenous P62 was revealed by immunofluorescence and total intensity of p62 were quantified on GFP positive cells by high content image acquisition and analysis. Raw Data represent mean values±s.e (n≥3); *, p<0.05. (D) Same as in (C) P62/actin ratios determined by immunoblotting (D) followed by densitometry (E). Data, means±s.e. (n≥3); *, p<0.05. (F) Proteolysis of proteins in HeLa. HeLa were transfected twice with control (scramble) or PNPLA5 siRNA, treated 20 hours with 500 μM BSA-Oleic Acid (OA) with media containing [³H] leucine and starved or not with or without Bafilomycin A1 (Baf) for 90 minutes. Leucine release was calculated from radioactivity in the tricarboxylic acid-soluble form relative to total cell radioactivity. Data, means±s.e. (n≥3); *, p<0.05 (G,H) Flow cytometry analysis of cellular mitochondrial content. HeLa were transfected twice with control (scramble) or PNPLA5 siRNA, treated 20 hours with 500 μM BSA-Oleic Acid (OA) and stained with MitoTracker Green. (G) histograms; (H) average mean fluorescence intensity (MFI) of MitoTracker Green per cell. Data, means±s.e. (n≥3); *, p<0.05. (I) Analysis of the role of PNPLA5 in autophagic killing of BCG. RAW 264.7 macrophages were transfected twice with PNPLA5 siRNAs or scramble (Scr) control. Cells were then treated 20 hours with 250 μM BSA-Oleic Acid (OA) and infected the day after with BCG. Autophagy was induced 4 hours by starvation (Starv). BCG survival (% of control BCG CFU) was analyzed and results shown represent mean±s.e.m. *, p<0.05.

FIG. 7 illustrates that lipid droplets contribute to autophagosome biogenesis.

### Increase in Cellular Lipid Droplet Content Increases Autophagic Capacity

Addition of oleic acid (OA) is commonly used to increase cellular LD content [22, 23]. We tested whether increase in LDs, following OA pretreatment (FIG. 1A) affected autophagy. Increase in LDs enhanced basal and induced autophagy, as determined by high content imaging analysis of autophagic organelles in HeLa cells stably expressing mRFP-GFP-LC3 (FIG. 1B-D). The enhancement was detected at both autophagy initiation stage (FIG. 1C; GFP-LC3) and autophagosomal maturation (FIG. 1D; mRFP-GFP), as determined by the

acidification-sensitive GFP and acidification-insensitive mRFP probes used to distinguish early autophagosomes and autolysosomes, respectively [25, 26]. The effect was confirmed by LC3-TI conversion immunoblot analyses (FIG. 1E,F). Titration experiments indicated that 500 μM OA used to induce formation of LDs was optimal for enhancing starvation-induced autophagy, as 1 mM OA caused either a plateau or potentially inhibitory effects on autophagy (FIG. 1E). The
latter effect was in keeping with reports that high concentrations of OA (e.g. 1 mM) can become inhibitory to autophagic maturation [27]. Hence, in subsequent experiments we used 500 μM OA. In keeping with the LC3 puncta assay, the standard bafilomycin A1 inhibition assay [28] showed (FIG. 1F) that pretreatment with OA enhanced cellular capacity for initiation of autophagy upon starvation used as a physiological inducer.
Early Autophagic Markers Associate with LDs

We next tested whether early autophagic factors, the mammalian Atg18 orthologs WIPI-1, WIPI-2B and WIPI-2D [29], associated with LDs induced by OA (FIG. 2A-J). Upon induction of autophagy by starvation, WIPI-1, WIPI-2B and WIPI-2D [29] were recruited to OA-induced LDs (FIG. 2A-F; compare to cells in Figure S1 incubated under basal conditions, i.e., in full medium). Atg18 and its orthologs are the only Atg factors known to bind to PI3P [29], a key phosphoinositide controlling autophagy [30]. In keeping with this, DFCP1, a PUP-binding protein and a marker for structures associated with autophagy precursors known as omegasomes [10], was also detected on LDs upon starvation (FIG. 2G, H). We also observed Atg16L1 on LDs by biochemical means but only before induction of autophagy by starvation (FIGS. 2I and J). These observations are in keeping with the reports that other autophagic factors such as Atg2 [31] and LC3 [6] localize with LDs. Thus, several well-characterized

early autophagosomal factors associate with LDs at different stages.

### Intravital Imaging Reveals Dynamic Interactions Between Autophagosomes and LD Organelles

Intravital imaging of mouse liver indicated vigorous interactions between LDs and LC3-positive autophagosomes in whole organs in live animal (FIG. 2K, Supplementary Movie 1). LD-autophagosome interactions fell under two categories: short range and long range (Supplementary Movie 1). The associations were transient in nature and appeared as “kiss-and-run” events between the two organelles. Thus, lipid droplets and autophagic organelles show intermittent short-lived interactions.

Consumption of LDs Continues when Either Autophagic Maturation or Autophagosome Closure are Blocked

Some, but not all, of the above observations could be interpreted as LDs being en route for lipophagy. To address this, autophagic maturation was blocked in cultured cells with bafilomycin A1. Under these conditions, LDs continued to be consumed during starvation, as quantified by high content automated imaging and analysis (Cellomics) of LD numbers per cell and total area of LDs (FIG. 3A-C). This phenomenon was further investigated using a dominant negative form of Atg4B, Atg4BC74A, which prohibits closure of nascent autophagosome [32). In these experiments, 3T3 cells stably expressing Atg4BC74A were used (32]. As a control, we first established that these cells did not support degradation of p62/sequestosome-1, often used as a readout for autophagic degradation (FIG. 3D,E). However, Atg4BC74A did permit continuing LD utilization during starvation (FIG. 3F,G). These results indicate that LDs are consumed during starvation-induced autophagy in processes other than autophagic degradation.

### Screen for TG-Mobilizing Enzymes Identifies PNPLA5 as a Positive Regulator of Autophagy

We wondered whether continuing LD consumption was due to a TO turnover in LDs independently of lipophagy. A potential intermediate, diacylglycerol (DAG) formed by lipase action upon TGs, could be used to build phospholipids necessary for autophagosomal membrane formation and growth. In considering this model, we first asked whether any of the TG metabolism enzymes including those mobilizing neutral lipid stores, such as the well-known adipose TG lipase, ATGL (PNPLA2), affected autophagy. The screen covered the TG-mobilization enzymes, represented by the papatin-like phospholipase domain containing

proteins, PNPLA1-5 (FIG. 4A and Figure S2A). It also included the TG biosynthetic enzymes, DGAT1 and 2 (FIG. 4A). Autophagy was assessed by high content imaging analysis (FIG. 4B-D). Knockdowns of DGAT1 and 2 did not affect LC3 puncta formation (FIG. 4C). However, knockdowns of PNPLAs with the exception of PNPLA1, reduced the numbers and the area of GFP-LC3 puncta under starvation conditions in OA-treated cells (FIG. 4D, Figure S2B,C).

Next, we focused on mammalian PNPLAs that yielded an LC3 puncta phenotype (FIG. 4D). The PNPLA family members have the following key properties. They contain a conserved lipase catalytic dyad (G-X-S-X-G) in the patatin domain (Figure S2A), which confers an in vitro lipase activity [33, 34]. Unlike other PNPLA members, PNPLA1 has no apparent TG-lipase activity and has been suggested to act in phospholipid metabolism instead of TG mobilization [35]. PNPLA2 (ATGL, adipose triglyceride lipase, also known under the name Desnutrin) is the best-studied TG-converting lipase responsible for most of TO hydrolysis in murine white adipose tissue [36, 37]. PNPLA3, also known as Adiponutrin can act as an acyltransferase [38] or a lipase [24], with its biosynthetic acyltransferase activity being the presumed dominant function [38].

Like PNPLA4, PNPLA5 has been shown to possess a lipase activity against TGs [24]. However, PNPLA5 shows some differences in its active site vs. PNPLA2, 3 and 4, suggesting further sub-specialization. Using LC3-11 immuno-blot assays in presence of Bafilomycin A1, we found that only a PNPLA5 knock-down inhibited LC3-II conversion (FIG. 4E, F). We then verified that PNPLA5 affected autophagy initiation by overexpressing PNPLA5-GFP construct in HeLa cells (FIG. 4G,H). By gating on GFP-positive cells (FIG. 4G), high content imaging analysis revealed that PNPLA5-expressing cells showed an increase in LC3 puncta numbers (FIG. 4H) and area (Figure S2D), measured by detecting endogenous LC3. In keeping with this newly uncovered role of PNPLA5 in autophagy initiation, PNPLA5 colocalized with ATG16L1 on LDs (FIG. 4I, J).

### CPT1 and LPCAT2 are Both Positive Regulator of Autophagy

If the products of PNPLA5 lipase action upon TGs [24] are used to build phospholipids (e.g. following enzymatic conversion of TGs to DAG) for autophagosomal membranes, we reasoned that the de novo synthesis of phosphatidylcholine (PC) or phosphoethanolamine (PE) may be needed to convert DAG to phospholipids during acute induction of autophagy. We focused on PC. Two major biochemical pathways contribute to the synthesis of PC: the Kennedy pathway for de novo PC synthesis with the participation of cholinephosphotransferase (CPT1; FIG. 4K) [39] and the Lands cycle for remodeling of the fatty acid composition of PC species through the concerted actions of phospholipase A2 (PLA2s) and lysophosphatidylcholine acyltransferases (LPCAT1 and 2; FIG. 4M) [40]. Thus, we tested whether CPT1 and LPCAT1/2 affected autophagy by high content imaging analysis of LC3 puncta (FIG. 4L,N). Knockdowns of CPT1 and LPCAT2 reduced the numbers and the area of GFP-LC3 puncta under starvation conditions in OAtreated cells (FIG. 4L,N; Figure S2E-H). Thus, these enzymes of the Kennedy pathway and the Lands cycle were important for LD-based enhancement of autophagic capacity. An efficient knockdown of LPCAT1 could not be obtained (Figure S3H) so we could not rule in or out whether LPCAT1, in addition to LPCAT2, contributes to optimal formation of autophagosomes. In conclusion, PNPLA5, which generates DAG, and CPT1 that transfers choline to the DAG to form PC, are both required for optimal initiation of autophagy. Furthermore, the PC remodeling pathway via LPCAT2, known to influence the formation of PC on LDs [41] also affects autophagy.

### Inhibition of Autophagosome Closure Increases Localization of DAG and Atg16L1 on LDs

The immediate product of PNPLAs as TG lipases is DAG. Using a DAG-specific GFP probe (NES-GFP-DAG; [42]), we tested whether DAG appeared on LDs in association with induction of autophagy by starvation (FIG. 5 and Figure S3). We furthermore tested DAG in relationship to an early autophagic marker, Atg16L1, due to the transient association of Atg16L1 with lipid droplets (FIGS. 2I and J). To trap such putative intermediates, we again employed cells expressing the dominant negative form of Atg4, Atg4BC74A [32], which prevents autophagosome closure and subsequent maturation events (Figure S and Figure S3).

The identity of LD) was established by LipidTOX-Red visualization (not shown). There was an increase (FIG. 5A) of DAG probe (Figure S3, green channel) on LDs in Atg4BC74A expressing cells (Figure S3; mStrawberry-Atg4BC74A is shown in the red channel) relative to mock vector containing cells. This reached statistical significance under autophagy inducing conditions by starvation (FIG. 5A). There was a statistically significant increase of Atg16L1on LDs (FIG. 5B) under autophagy inducing conditions (Atg16L1 is shown as blue channel in Figure S3) in Atg4BC74A-expressing cells. A trend in increase of co-localization of Atg16L1 and DAG on LDs was observed when autophagosomal completion was blocked by Atg4BC74A (FIG. 5C,D). An increase in DAGATG16L1 co-localization was statistically significant when mock cells in basal (fed) conditions were compared to Atg4BC74A-expressing cells under autophagy inducing conditions (FIG. 5C,D). These observations indicate that DAG, detected by the NES-GFP-DAG probe, was associated with an autophagy initiation marker on LDs upon induction of autophagy by starvation, and that these intermediates were trapped by blocking autophagosomal progression.

### PNPLA5 is Involved in Autophagy of Diverse Cargoes

The above observations collectively indicate that autophagy initiation is associated with LDs, and that LDs support generation of autophagosomes. Autophagosomes derived in part from LDs could be specializing in lipophagy. Alternatively, autophagosomes originating at or from LDs might target other autophagic substrates. We used PNPLA5 knock-downs to differentiate between these two possibilities. PNPLA5 knock-down inhibited LDs consumption (FIG. 6A,B, Figure S4). When we tested other autophagy substrates, it turned out that PNPLA5 affected magnitude of these processes as well. PNPLA5 knock-down

inhibited degradation of the autophagic adaptor, Sequestosome-1/p62, as one of conventional reporters of selective autophagy (FIG. 6C). PNPLA5 was required for optimal proteolysis, since PNPLA5 knockdown reduced autophagy-dependent (i.e. bafilomycin A1-inhabitable component) of proteolysis (FIG. 6D). Mitophagy decreased in cells subjected to PNPLA5 knockdown, as shown by increase in mitochondrial content per cell measured by MitoTracker Green (FIG. 6E). Finally, elimination of an intracellular microbe (Mycobacterium bovis BCG) by xenophagy was reduced upon PNPLA5 knockdown (FIG. 6F). These findings demonstrate that PNPLA5 plays a role in autophagy of diverse substrates including an autophagic adaptor-mediated processes, organelles (mitophagy), bacteria (xenophagy) and bulk autophagy of the cytosol, and suggests a model in which autophagy initiation at sites controlled by PNPLA5 (e.g. LDs as shown here) affects autophagy in general and not just the autophagy engaged in lipophagy.

This work shows that lipid droplets, as intracellular neutral lipid stores, enhance autophagic capacity of a mammalian cell. A build up in lipid droplets prior to induction of autophagy enables increased autophagosomal formation in response to starvation. This enhancement of autophagic capacity depends on PNPLA5, a member of the papatin-like phospholipase domain-containing proteins that mobilize neutral lipids. In addition to mobilization of neutral lipids, an enzyme, CPT1, important for de novo phospholipid synthesis, and an enzyme engaged in PC remodeling, LPCAT2, are needed for lipid droplet-dependent

enhancement of autophagy. Taken together, these results indicate that lipid droplets as stores of neutral lipids [22, 23] represent a contributing source of membrane precursors for formation of autophagosomes (FIG. 7). By mobilizing the precipitated out lipid matter, i.e. the TGs within LDs cells are able to safely build new autophagosomes without unnecessarily compromising integrity and functionality of the pre-existing organelles.

### Materials and Methods

### Cell Culture and Plasmids

Human HeLa and mouse macrophage-like cell line RAW 264.7 were from ATCC. U2OS cells stably expressing EGFP-WIPI-1, EGFP-WIPI-2B and EGFP-WIPI-2D were generated in T. Proikas-Cezanne laboratory. NIH3T3 cells stably expressing Atg4B or mStrawberry-Atg4BC74A are from T. Yoshimori (Osaka, Japan). HEK-293 stably expressing GFP-DFCP1 and HeLa cells stably expressing mRFP-GFP-LC3 were respectively from N. Ktistakis (Cambridge, UK) and D. Rubinsztein (Cambridge, UK). Plasmid expressing NES-GFP-DAG was from T. Balla (NIH Bethesda, USA). Human PNPLA5-GFP construct was generated in this work.

### Pharmacological Agonists, Inhibitors, and Autophagy

Cells were treated with 100 nM bafilomycin A1 (LC Laboratories) and autophagy was induced for indicated times by starvation in EBSS (Sigma-Aldrich).

### Proteolysis of Proteins

HeLa cells were transfected twice with siRNA and 4 h following the second

transfection, proteins were radiolabeled by incubation in media containing 1 μCi/ml [3H] leucine. Following 20 h of radiolabeling, cells were incubated in full or starvation media with or without bafilomycin A1 for 90 min. Trichloroacetic acid (TCA)-precipitable radioactivity in the cells monolayers and the TCA-soluble radioactivity released into the media were determined. Leucine release (a measure of proteolysis) was calculated as a ratio between TCA-soluble supernatant and total cell-associated radioactivity.

### Mycobacterial Survival

Microbiological analyses of bacterial viability (M. bovis BCG) were carried out as previously described [59].

### Oleic Acid and Free Fatty-Acid BSA

Oleic acid (Sigma-Aldrich) was complexed at room temperature with fatty acid-free bovine serum albumin as previously described [60].

Knockdowns with siRNAs and Knockdown Validation

HeLa, NIH3T3 and RAW 264.7 cells were transfected by nucleoporation using

Nucleofector Reagent Kit R, V and V respectively (Amaxa/Lonza biosystems).
Non-targeting siRNA pool (Scrambled) was used as a control. Knockdown validation was carried out either by immunoblotting or quantitative RT-PCR. SMARTpool SiGENOME siRNAs used in this study and RT-PCR primers used for knockdown validation are listed in Supplementary Table S1.

### Quantitative RT-PCR

Total RNA was extracted from HeLa and cDNA was synthesized using a Cells-to-Ct Kit (Applied Biosystems), according to the manufacturer's instructions. Real time PCR was performed using SYBR Green Master Mix (Applied Biosystems), and products were detected on a Prism 5300 detection system (SDS, ABI/Perkin-Elmer). The relative extent of DGAT1, DGAT2, PNPLA1, PNPLA2, PNPLA3, PNPLA4, PNPLA5 expression was calculated using the 2eΔΔC(t) method. Conditions for real time PCR were: initial denaturation for 10 min at 95° C.,

followed by amplification cycles with 15 s at 95° C. and 1 min at 60° C.

### Antibodies, Immunoblotting, Detection Assays, and Conventional Microscopy

Blots were analyzed with antibodies to LC3 (Sigma), P62 (BD Transduction),

Actin (Sigma), Atg16L1 (Cosmo Bio), Adipophilin (Progen Biotechnik), LPCAT1 (ProteinTech Group Inc), LPCAT2 (Novus Biologicals); staining was revealed with Super Signal West Dura chemiluminescent substrate (Pierce). Immunofluorescence confocal microscopy was carried out using a Zeiss LSM 510 Meta microscope (laser wavelengths 488 nm, 543 nm and 633 nm). Antibodies against endogenous proteins LC3 (MBL), P62 (BD Transduction), Atg16L1 (Cosmo Bio) were used for indirect immunofluorescence analysis. To preserve lipid droplet structure, immunofluorescence were performed as previously described [61]. SlideBook morphometric analysis software 5.0 (Intelligent Imaging Innovations) was used to quantify the colocalization between Atg16L1 or DAG and lipid droplets. Percentage of lipid droplet-marker colocalization was fraction of total lipid droplet examined scored as positive when one or more puncta or homogeneously distributed marker were observed overlapping with the mask of lipid droplet (derived from a dilatation at 110% of the area of lipid droplet). Data are from 3 independent experiments in which, each time, more than 300 lipid droplet were analysed. Pearson's colocalization coefficients were also derived using Slide Book 5.0. Pearson's coefficient was from three independent experiments with five fields per experiment for a total of 15 fields contributing to the cumulative result.

### High Content Image Acquisition and Analysis

Cellomics Array Scan (Thermo Scientific) was used to acquire images by computer-driven (operator independent) collection of 49 valid fields per well with cells in 96 well plates, with >500 cells (identified by the program as valid primary object) per each sample. Objects were morphometrically and statistically analyzed using the iDev software (Thermo Scientific). Computer-driven identification of primary and secondary objects was based on predetermined

parameters, and fluorescent objects (cells, puncta, droplets, total cytoplasm) were quantified using a suite of applicable parameter (including number of objects per cell; total area per cell; total intensity). GFP fluorescent puncta or endogenous LC3 and p62 were revealed by fluorescent antibody staining. Bodipy 493/503, LipidTOX™ Red and LipidTOX™ DeepRed (Molecular Probes) were used to stain lipid droplets.

### Subcellular Fractionation

Subcellular membranous organelles were separated by isopycnic centrifugation

in sucrose gradients as described [62]. Cells were homogenized in 250 mM sucrose, 20 mM HEPES-NaOH pH 7.5, 0.5 mM EGTA, post nuclear supernatant
layered atop of pro-formed 60-15% sucrose gradients, and samples centrifuged
at 100,000 g in a Beckman SW 40 rotor for 18 h at 4° C. Equivalent density
fractions (verified for refractive index match) were analyzed by immunoblotting.

### Mitotracker Staining and Flow Cytometry

HeLa cells were stained with 300 nM of Mitotracker Green during 15 minutes at

37° C. Flow cytometry was carried out on the LSRFortessa (BD Biosciences) and data analyzed using FlowJo software (TreeStar).

### Intravital Imaging

All experiments were approved by the National Institute of Dental and Craniofacial Research (NIDCR, National Institute of Health, Bethesda, Md., USA) Animal Care and Use Committee. LC3-GFP mice [63] were fed ad libitum prior to the procedure. The animals were anesthetized with a mixture of ketamine and xylazine as described in Masedunskas at al., 2011 [64]. The liver was exposed by performing a transversal incision (1 cm×1 cm) in the right side of the abdomen just below the diaphragm. The exposed organ was bathed for 30 minutes with Bodipy 665 positioned on the pre-warmed stage of an Olympus Fluoview 1000 (Masedunskas at al., 2008). The temperature of the body and the organ were continuously monitored and maintained with a heat lamp. Time lapse-imaging was performed by confocal microscopy (Excitation for GFP: 488 nm; Excitation for Bodipy 665: 561 nm), as previously described (Masedunskas t al., 2011).

### REFERENCES FOR EXAMPLE 1

1. Mizushima, N., Yoshimori, T., and Ohsumi, Y. (2011). The role of Atg proteins in autophagosome formation. Annu Rev Cell Dev Biol 27, 107-132.
2. Mizushima, N., and Komatsu, M. (2011). Autophagy: renovation of cells and tissues. Cell 147, 728-741.
3. Levine, B., Mizushima, N., and Virgin, H. W. (2011). Autophagy in immunity and inflammation. Nature 469, 323-335.
4. Rubinsztein, D. C., Codogno, P., and Levine, B. (2012). Autophagy modulation as a potential therapeutic target for diverse diseases. Nat Rev Drug Discov 11, 709-730.
5. Youle, R J., and Narendra, D. P. (2011). Mechanisms of mitophagy. Nat Rev Mol Cell Biol 12, 9-14.
6. Singh, R., Kaushik, S., Wang, Y., Xiang. Y., Novak, L, Komatsu, M., Tanaka, K., Cuervo, A. M., and Czaja, M. J. (2009). Autophagy regulates lipid metabolism. Nature 458, 1131-1135.
7. Xie, Z., and Klionsky, D. J. (2007). Autophagosome formation: core machinery and adaptations. Nat Cell Biol 9, 1102-1109.
8. Itakura, E., Kishi-Itakura, C., and Mizushima, N. (2012). The hairpin-type tail-anchored SNARE syntaxin 17 targets to autophagosomes for fusion with endosomes/lysosomes. Cell 151, 1256-1269.
9. He, C., and Klionsky, D. J. (2009). Regulation Mechanisms and Signaling Pathways of Autophagy. Annu Rev Genet.
10. Axe, E. L., Walker, S. A., Manifava, M., Chandra, P., Roderick, H. L., Habermann, A., Griffiths, G., and Ktistakis, N. T. (2008). Autophagosome formation from membrane compartments enriched in phosphatidylinositol
3-phosphate and dynamically connected to the endoplasmic reticulum. The Journal of cell biology 182, 685-701.
11. Hayashi-Nishino, M., Fujita, N., Noda, T., Yamaguchi, A., Yoshimori, T., and Yamamoto, A. (2009). A subdomain of the endoplasmic reticulum forms a cradle for autophagosome formation. Nat Cell Biol 11, 1433-1437.
12. Yla-Anttila, P., Vihinen, H., Jokitalo, E., and Eskelinen, E. L. (2009). 3D tomography reveals connections between the phagophore and endoplasmic reticulum. Autophagy 5f, 1180-1185.
13. Proikas-Cezanne, T., Waddell, S., Gaugel, A., Frickey, T., Lupas, A., and
Nordheim, A. (2004). WIPI-1alpha (WIPI49), a member of the novel 7-bladed WIPI protein family, is aberrantly expressed in human cancer and is linked to starvation-induced autophagy. Oncogene 23, 9314-9325.
14. Poison, H. E., de Lartigue, J., Rigden, D. J., Reedijk, M., Urbe, S., Clague, M. J., and Tooze, S. A. (2010). Mammalian Atg18 (WIPI2) localizes to omegasome-anchored phagophores and positively regulates LC3 lipidation. Autophagy 6.
15. Rubinsztein, D. C., Shpilka, T., and Elazar, Z. (2012). Mechanisms of autophagosome biogenesis. Curr Biol 22, R29-34.
16. Tooze, S. A., and Yoshimori, T. (2010). The origin of the autophagosomal membrane. Nat Cell Biol 12, 831-835.
17. Hamasaki, M., Furuta, N., Matsuda, A., Nowu, A., Yamamoto, A., Fujita, N., Oomori, H., Noda, T., Haraguchi, T., Hiraoka, Y., et al. (2013).
Autophagosomes form at ER-mitochondria contact sites. Nature.
18. Hailey, D. W., Rambold, A. S., Satpute-Krishnan, P., Mitra, K., Sougrat, R., Kim, P. K., and Lippincott-Schwartz, J. (2010). Mitochondria supply membranes for autophagosome biogenesis during starvation. Cell 141, 656-667.
19. van der Vaart, A., Griffith, J., and Reggiori, F. (2010). Exit from the Golgi is required for the expansion of the autophagosomal phagophore in yeast Saccharomyces cerevisiae. Molecular biology of the cell 21, 2270-2284.
20. Yen, W. L., Shintani, T., Nair, U., Cao, Y., Richardson, B. C., Li, Z., Hughson, F. M., Baba, M., and Klionsky, D. J. (2010). The conserved oligomeric Golgi complex is involved in double-membrane vesicle formation during autophagy. The Journal of cell biology 188, 101-114.
21. Ravikumar, B., Moreau, K., Jahreiss, L., Puri, C., and Rubinsztein, D. C.
(2010). Plasma membrane contributes to the formation of preautophagosomal structures. Nat Cell Biol 12, 747-757.
22. Fujimoto, T., and Patton, R. G. (2011). Not just fat: the structure and function of the lipid droplet. Cold Spring Harbor perspectives in biology 3.
23. Walther, T. C., and Farese, R. V., Jr. (2012). Lipid droplets and cellular lipid metabolism. Annual review of biochemistry 81, 687-714.
24. Lake, A. C., Sun, Y., Li, J. L, Kim, J. E., Johnson, J. W., Li, D., Revett, T., Shih, H. H., Liu, W., Paulsen, J. E., et al. (2005). Expression, regulation, and triglyceride hydrolase activity of Adiponutrin family members. Journal of lipid research 46, 2477-2487.
25. Kimura, S., Noda, T., and Yoshimori, T. (2007). Dissection of the autophagosome maturation process by a novel reporter protein, tandem fluorescent-tagged LC3. Autophagy 3, 452-460.
26. Pankiv, S., Clausen, T. H., Lamark, T., Brech, A., Bruun, J. A., Outzen, H., Overvatn, A., Bjorkoy, G., and Johansen, T. (2007). p62/SQSTM1 binds directly to Atg8/LC3 to facilitate degradation of ubiquitinated protein aggregates by autophagy. J Biol Chem 282, 24131-24145.
27. Koga, H., Kaushik, S., and Cuervo, A. M. (2010). Inhibitory effect of intracellular lipid load on macroautophagy. Autophagy 6, 825-827.
28. Mizushima, N., Yoshimori, T., and Levine, B. (2010). Methods in mammalian autophagy research. Cell 140, 313-326.
29. Proikas-Cezanne, T., Ruckerbauer, S., Stierhof, Y. D., Berg. C., and Nordheim, A. (2007). Human WIPI-1 puncta-formation: a novel assay to assess mammalian autophagy. FEBS Lett 581, 3396-3404.
30. Petiot, A., Ogier-Denis, E., Blommaart, E. F., Meijer, A J., and Codogno, P. (2000). Distinct classes of phosphatidylinositol 3′-kinases are involved in signaling pathways that control macroautophagy in HT-29 cells. J Biol Chem 275, 992-998.
31. Velikkakath, A. K., Nishimura, T., Oita, E., Ishihara, N., and Mizushima, N. (2012). Mammalian Atg2 proteins are essential for autophagosome formation and important for regulation of size and distribution of lipid droplets. Molecular biology of the cell 23, 896-909.
32. Fujita, N., Hayashi-Nishino, M., Fukumoto, H., Omori, H., Yamamoto, A.,
Noda, T., and Yoshimori, T. (2008). An Atg4B mutant hampers the lipidation of LC3 paralogues and causes defects in autophagosome closure. Molecular biology of the cell 19, 4651-4659.
33. Lass, A., Zimmermann, R., Oberer, M., and Zechner, R. (2011). Lipolysis—a highly regulated multi-enzyme complex mediates the catabolism of cellular fat stores. Prog Lipid Res 50, 14-27.
34. Rydel, T. J., Williams, J. M., Krieger, E., Moshiri, F., Stallings, W. C., Brown, S. M., Pershing, J. C., Purcell, J. P., and Alibhai, M. F. (2003). The crystal structure, mutagenesis, and activity studies reveal that palatin is a lipid acyl hydrolase with a Ser-Asp catalytic dyad. Biochemistry 42, 6696-6708.
35. Grail, A., Guaguere, E., Planchais, S., Grond, S., Bourrat, E., Hausser, I., Hitte, C., Le Gallo, M., Derbois, C., Kim, G J., et al. (2012). PNPLA1 mutations cause autosomal recessive congenital ichthyosis in golden retriever dogs and humans. Nature genetics 44, 140-147.
36. Schweiger, M., Schreiber, R., Haemmerle, G., Lass, A., Fledelius, C.,
Jacobsen, P., Tornqvist, H., Zechner, R., and Zimmermann, R. (2006). Adipose triglyceride lipase and hormone-sensitive lipase are the major enzymes in adipose tissue triacylglycerol catabolism. J Biol Chem 281, 40236-40241.
37. Smirnova, E., Goldberg. E. B., Makarova, K. S., Lin, L., Brown, W J., and Jackson, C. L. (2006). ATGL has a key role in lipid droplet/adiposome degradation in mammalian cells. EMBO Rep 7, 106-113.
38. Kumari, M., Schoiswohl, G., Chitraju, C., Paar, M., Cornaciu, I., Rangrez, A. Y., Wongsiriroj, N., Nagy, H. M., Ivanova, P. T., Scott, S. A., et al. (2012). Adiponutrin functions as a nutritionally regulated lysophosphatidic acid acyltransferase. Cell metabolism 15, 691-702.
39. Gibellini, F., and Smith, T. K. (2010). The Kennedy pathway—De novo synthesis of phosphatidylethanolamine and phosphatidylcholine. IUBMB Life 62, 414-428.
40. Shindou, H., Hishikawa, D., Harayama, T., Yuki, K., and Shimizu, T. (2009). Recent progress on acyl CoA: lysophospholipid acyltransferase research. Journal of lipid research 50 Suppl, S46-51.
41. Moessinger, C., Kuerschner, L., Spandl, J., Shevchenko, A., and Thiele, C. (2011). Human lysophosphatidylcholine acyltransferases 1 and 2 are located in lipid droplets where they catalyze the formation of phosphatidylcholine. J Biol Chem 286, 21330-21339.
42. Kim, Y. J., Guzman-Hernandez, M. L., and Balla, T. (2011). A highly dynamic ER-derived phosphatidylinositol-synthesizing organelle supplies phosphoinositides to cellular membranes. Dev Cell 21, 813-824.
43. Robenek, H., Hofnagel, O., Buers, I., Robenek, M. J., Troyer, D., and Severs, N. J. (2006). Adipophilin-enriched domains in the ER membrane are sites of lipid droplet biogenesis. J Cell Sci 119, 4215-4224.
44. Bodemann, B. O., Orvedahl, A., Cheng, T., Ram, R. R., Ou, Y. H.,
Formstecher, E., Maiti, M., Hazelett, C. C., Wauson, E. M., Balakireva, M., et al. (2011). RalB and the exocyst mediate the cellular starvation response by direct activation of autophagosome assembly. Cell 144, 253-267.
45. Dupont, N., Jiang, S., Pilli, M., Ornatowski, W., Bhattacharya, D., and Deretic, V. (2011). Autophagy-based unconventional secretory pathway for extracellular delivery of IL-1 beta. The EMBO journal 30, 4701-4711.
46. Zehmer, J. K., Bartz, R., Bisel, B., Liu, P., Seemann, J., and Anderson, R. G. (2009). Targeting sequences of UBXD8 and AAM-B reveal that the ER has a direct role in the emergence and regression of lipid droplets. J Cell Sci 122, 3694-3702.
47. Tarnopolsky, M. A., Rennie, C. D., Robertshaw, H. A., Fedak-Tarnopolsky, S. N., Devries, M. C., and Hamadeh, M. J. (2007). Influence of endurance exercise training and sex on intramyocellular lipid and mitochondrial ultrastructure, substrate use, and mitochondrial enzyme activity. Am J Physiol Regul Integr Comp Physiol 292, R1271-1278.
48. Moreau, K., Ravikumar, B., Renna, M., Purl, C., and Rubinsztein, D. C. (2011). Autophagosome precursor maturation requires homotypic fusion. Cell 146, 303-317.
49. Pilli, M., Arko-Mensah, J., Ponpuak, M., Roberts, E., Master, S., Mandell,
M. A., Dupont, N., Ornatowski, W., Jiang, S., Bradfute, S. B., et al. (2012). TBK-1 Promotes Autophagy-Mediated Antimicrobial Defense by Controlling Autophagosome Maturation. Immunity 37, 223-234.
50. Liu, P., Bartz, R., Zehmer, J. K., Ying, Y. S., Zhu, M., Serrero, G., and Anderson, R. G. (2007). Rab-regulated interaction of early endosomes with lipid droplets. Biochim Biophys Acta 1773, 784-793.
51. Velikkakath, A. K., Nishimura, T., Oita, E., Ishihara, N., and Mizushima, N. (2012). Mammalian Atg2 proteins are essential for autophagosome formation and important for regulation of size and distribution of lipid droplets. Mol Biol Cell 23, 896-909.
52. Kaushik, S., Rodriguez-Navarro, J. A., Arias, E., Kiffin, R., Sahu, S., Schwartz, G. J., Cuervo, A. M., and Singh, R. (2011). Autophagy in hypothalamic AgRP neurons regulates food intake and energy balance. Cell metabolism 14, 173-183.
53. Hubbard, V. M., Valdor, R., Patel, B., Singh, R., Cuervo, A. M., and Macian, F. (2010). Macroautophagy regulates energy metabolism during effector T cell activation. Journal of immunology 185, 7349-7357.
54. Martinez-Vicente, M., Talloczy, Z., Wong, E., Tang, G., Koga, H., Kaushik,
S., de Vries, R., Arias, E., Harris, S., Sulzer, D., et al. (2010). Cargo
recognition failure is responsible for inefficient autophagy in Huntington's
disease. Nat Neurosci 13, 567-576.
55. Chen, H. C., Stone, S. J., Zhou, P., Buhman, K. K., and Farese, R. V., Jr. (2002). Dissociation of obesity and impaired glucose disposal in mice overexpressing acyl coenzyme a:diacylglycerol acyltransferase 1 in white adipose tissue. Diabetes 51, 3189-3195.
56. Franckhauser, S., Munoz, S., Pujol, A., Casellas, A., Riu, E., Otaegui, P., Su, B., and Bosch, F. (2002). Increased fatty acid re-esterification by PEPCK overexpression in adipose tissue leads to obesity without insulin resistance. Diabetes 51, 624-630.
57. Singh, R., and Cuervo, A. M. (2012). Lipophagy: connecting autophagy and lipid metabolism. International journal of cell biology 2012, 282041.
58. Das, S. K., Eder, S., Schauer, S., Diwoky, C., Temmel, H., Guertl, B., Gorkiewicz, G., Tamilarasan, K. P., Kumari, P., Trauner, M., et al. (2011). Adipose triglyceride lipase contributes to cancer-associated cachexia. Science 333, 233-238.
59. Ponpuak, M., Davis, A. S., Roberts, E. A., Delgado, M. A., Dinkins, C.,
Zhao, Z., Virgin, H. W. t., Kyei, G. B., Johansen, T., Vergne, I., at al. (2010). Delivery of cytosolic components by autophagic adaptor protein p62 endows autophagosomes with unique antimicrobial properties. Immunity 32, 329-341.
60. Brasaemle, D. L., and Wolins, N. E. (2006). Isolation of lipid droplets from cells by density gradient centrifugation. Curr Protoc Cell Biol Chapter 3, Unit 3 15.
61. Listenberger, L. L., and Brown, D. A. (2007). Fluorescent detection of lipid droplets and associated proteins. Curr Protoc Cell Biol Chapter 24, Unit 24 22.
62. Singh, S. B., Ornatowski, W., Vergne, I., Naylor, J., Delgado, M., Roberts, E., Ponpuak, M., Master, S., Pilli, M., White, E., et al. (2010). Human IRGM regulates autophagy and cell-autonomous immunity functions through mitochondria. Nat Cell Biol 12, 1154-1165.
63. Mizushima, N., Yamamoto, A., Matsui, M., Yoshimori, T., and Ohsumi, Y. (2004). In vivo analysis of autophagy in response to nutrient starvation using transgenic mice expressing a fluorescent autophagosome marker. Mol Biol Cell 15, 1101-1111.
64. Masedunskas, A., Sramkova, M., Parente, L., Sales, K. U., Amornphimoltham, P., Bugge, T. H., and Weigert, R. (2011). Role for the actomyosin complex in regulated exocytosis revealed by intravital microscopy. Proceedings of the National Academy of Sciences of the United States of America 108, 13552-13557.

### Example 2

### Lipid Effect on Autophagy

Figure S1 shows the absence of WIPI colocalization with lipid droplets under basal conditions. The panel (A-D) images show confocal microscopy analysis of U2OS cells stably expressing GFP WIPI-1 (A), GFP WIPI-2B (B) and GFP WIPI-2D (C).

Fluorescence shows lipid droplets (blue, LipidTox DoepRed). Cells were treated for 20 h with 500 μM BSA-Oleic Acid (OA) and then analyzed by confocal fluorescence microscopy.

Figure S2 shows an analysis of triglyceride mobilizing factors PNPLAs, Kennedy biosynthetic cycle and Lands remodeling cycle enzymes in lipid droplet

contribution to the cellular autophagic capacity (A) Members of the catalytically active papatin-like phospholipese domain containing proteins. PNPLA1-5. (B,C) Stable mRFP-GFP-LC3 HeLa cells were transfected twice with scramble (Scr) control siRNA or siRNAs against PNPLA1,
PNPLA2, PNPLA3, PNPLA4, and PNPLA5. After 24 h following transfection,
cells were treated for 20 h with BSA alone or with 500 μM BSA-Oleic Acid (OA) and starved in EBSS or not (full medium) for 90 min. Graph in B, an example of raw data (number of GFP-LC3 dots per cell) from a single high content analysis experiment (data in the main sequence figures include data from 3 or more independent experiments as shown here). Graph in C, total area of GFP+ puncta were quantified by high content image acquisition and analysis. Data represent mean values±s.e. (n≥3); *, p<0.05. (D) Effect of PNPLA5 overexpression on autophagy induction by quantifying total area of endogenous LC3 dots. HeLa cells were transfected either with GFP or PNPLA5-GFP expressing plasmids, then treated 20 h with 500 μM BSA-Oleic Acid (OA).

Next, cells were starved for 2 h with or without Bafilomycin A1 (Baf). Endogenous LC3 was stained by immunofluorescence and total area of LC3 dots were quantified in GFP-positive cells by high content image acquisition and analysis. Data represent mean values±s.e. (n≥3); *, p<0.05. (E,F) HeLa cells stably expressing mRFP-GFP-LC3 were transfected twice with scrambled (Scr) siRNA or siRNAs against CPT1 (siRNA details are given in Supplementary Table 1). After 24 h post-transfection, cells were treated for 20 h with 500 μM BSA-Oleic Acid (OA) and starved or not for 90 min. GFP+ puncta per cell were quantified by high content image acquisition and analysis. Data in E, number of GFP+ puncta per cell. Data in F, total area of GFP+ puncta per cell. Data represent mean values±s.e. (n≥3); *, p<0.05. (G) HeLa cells stably expressing mRFP-GFP-LC3 were transfected for scrambled (Scr) control siRNA or siRNAs against LPCAT1, LPCAT2 (details of siRNAa are in Supplementary Table 1). After 48 h of transfection, cells were treated for 20 h with 500 μM BSA-Oleic Acid (OA) and starved in EBSS or not (incubated in full medium) for 90 min. GFP+ puncta per cell were quantified by high content image acquisition and analysis. (H) HeLa cells were transfected with scrambled control siRNA (Scr) or siRNAs against LPCAT1 and LPCAT2. After 48 h of transfection, cells were incubated for 20 h with 500 μM BSA-Oleic Acid (OA) and LPCAT1 and LPCAT2 levels assayed by immunoblots.

Supplementary Table S1 shows the description of enzymes involved and their
respective siRNA Dharmacon catalog number used in this study.

| | | | | | SMARTpool | Knockdown |
|---|---|---|---|---|---|---|
| | | | | | SIGENOME | verified by |
| Enzyme | | Full | Alternative | NCKI | Dharmacon | (min |
| name | Organism | Name | names | Gene # | CAT# | 50% KD) |
| DGAT1 | Human | dacylglycerol | | 8694 | M-003922-02-005 | RT-PCR |
| | | O-acrytransferase 1 | | | | |
| DGAT2 | Human | dacylglycerol | | 84649 | M-009333-00-005 | RT-PCR |
| | | O-acrytransferase 1 | | | | |
| PNPLA1 | Human | patatin-like | | 285848 | M-009042-01-0005 | RT-PCR |
| | | phospholipase domain | | | | |
| | | containing 1 | | | | |
| PNPLA2 | Human | patatin-like | ATGL, TTS-2 | 57104 | M-009003-01-005 | RT-PCR |
| | | phospholipase domain | 2, Desnutrin | | | |
| | | containing 2 | | | | |
| PNPLA3 | Human | patatin-like | Adiponutrin | 80339 | M-009564-01-005 | RT-PCR |
| | | phospholipase domain | | | | |
| | | containing 3 | | | | |
| PNPLA4 | Human | patatin-like | GS2 | 8228 | M-010271-01-0005 | RT-PCR |
| | | phospholipase domain | | | | |
| | | containing 4 | | | | |
| PNPLA5 | Human | patatin-like phospholipase | GS2L | 150379 | M-009563-01-005 | RT-PCR |
| | | domain containing 5 | | | | |
| PNPLA5 | Mouse | patatin-like phospholipase | GS2L | 75772 | M-048942-01-005 | RT-PCR |
| | | domain containing 5 | | | | |
| LPCAT1 | Human | Lysophosphatidylcholine | | 79888 | M-010289-00-005 | WB |
| | | acyltransferase 1 | | | | |
| LPCAT2 | Human | Lysophosphatidylcholine | | 54947 | M-010285-00-005 | WB |
| | | acyltransferase 2 | | | | |
| CPT1 | Human | Choline phosphotransferase | CHPT1 | 56994 | M-009775-02-005 | ND |

Supplementary Table S2 below shows the primer sequence used in this study,

| Sequence Name | Sequence |
|---|---|
| Actin-F | AAG ACC TGT ACG CCA ACA CA |
| Actin-R | TGA TCT CCT TCT GCA TCC TG |
| DGAT1-F | TCA AGT ATG GCA TCC TGG TG |
| DGAT1-R | AAG ACA TTG GCC GCA ATA AC |
| DGAT2-F | TCC AGC TGG TGA AGA CAC AC |
| DGAT2-R | TGT GCT GAA GTT GCA GAA GG |
| PNPLA1-F | CCA GAT AGA ACT CGC CCT TG |
| PNPLA1-R | GTG AGG TTG TGT GGC TCC TT |
| PNPLA2-F | CAA CAC CAG CAT CCA GTT CA |
| PNPLA2-R | ATC CCT GCT TGC ACA TCT CT |
| PNPLA3-F | ATG TCC ACC AGC TCA TCT CC |
| PNPLA3-R | GCA TCC ACG ACT TCG TCT TT |
| PNPLA4-F | AGA ACC GAC TGC ACG TAT CC |
| PNPLA4-R | TGC TGG CTA GGA GGA CCT TA |
| PNPLA5-F | TCC TGG GGC TCA TAT GTC TC |
| PNPLA5-R | AGT CCA CGT CTC TCC AGG AA |

Figure S3 shows the imaging of DAG and Atg16L1 co-localization. Panal (A-D): 3T3 cells expressing Atg4B (A,B) or mStrawberry-Atg4BC74A (mSberry Atg4B C74A; C,D) were transfected with a plasmid expressing NES-GFP-DAG, treated for 20 h with 500 μM BSA-Oleic Acid, and starved in EBSS (B,D) or not (A,C; full medium) for 2.5 h. Cells were then fixed and labeled with Atg16L1 antibody. Line in D corresponds to line tracing in FIG. 5.

Figure S4 shows that PNPLA5 knockdown inhibits lipid droplets consumption upon induction of autophagy by starvation. (A) Fluorescent images from high content image acquisition and analysis. Effect of PNPLA5 knockdown on lipid droplet degradation upon autophagy induction. HeLa cells were transfected twice with siRNAs PNPLA5 or scramble (Scr) control. Cells were then treated for 20 h with BSA or with 500 μM BSA-Oleic Acid

(OA) and starved or not for 2 h. Cells were then fixed and lipid droplets stained
for immunofluorescence with Bodipy 493/503.

### Example 3

### TRIM Protein Regulate Autophagy

Certain figure citations in this example have the format “Figure X/Y, Z”. This means Figure X, Panals Y and Z. For example, “FIG. 1A/D, E” means FIG. 1A, Panals D and E.

We employed a high-content image analysis (FIG. 1A and Figure S1A) with the autophagosomal marker LC3 (Kabeya et al., 2000; Mizushima et al., 2010) to screen the effects on autophagy of TRIM knockdowns (FIG. 1B-C and Figure S1A/B). Two conditions were examined, autophagy induced with the mTOR inhibitor pp242 (FIG. 1A/A, B) and basal autophagy (FIG. 1A/D, E). Knockdown of twenty-one TRIMs reduced GFP-LC3B puncta area (FIG. 1B) or puncta numbers (Figure S1A/B) per cell under induced conditions, comparably to a knockdown of Beclin 1. Ten TRIMs showed a converse effect. Additional TRIMs affected basal autophagy (FIG. 1A/D, E). Thus, a large fraction of TRIMs positively and negatively regulate autophagy.

Referring to FIG. 1A/A HeLa cells stably expressing mRFP-GFP-LC3B were subjected to TRIM knockdowns, treated with pp242, and imaged to detect nuclear stain (blue) and GFP signal (green). Top, non-targeting siRNA-transfected cells treated with carrier (DMSO) or pp242. White lines, cell borders. Red, LC3B puncta borders. Bottom, representative images of cells subjected to knockdown of TRIM45 and TRIM2, both treated with pp242. (B) Measurement of average area of GFP-LC3B area per cell from cells treated as in (A) (data from multiple 96-well plates with identical siRNA arrangements represent means and ±SE).

Encircled are pp242-induced wells (pp242, right) and wells with vehicle controls (DMSO, left bottom). TRIM knockdowns that reduced or increased LC3B puncta readout by 3 SD (horizontal lines) from pp242-treated controls are indicated by corresponding TRIM numbers. Gray point (Bec), Beclin 1 knockdown; red point (5), TRIM5α. (C) Domain organization of TRIM sub-families (I-XI; UC, unclassified). TRIM hits (LC3 puncta area >3 SD cutoff). Domain functions: RING (or R), E3-ligase domain; BB1 and BB2, protein-protein interactions; CCD, protein-protein interactions; COS, microtubule binding; SPRY, protein-protein interactions; FN3, DNA or heparin binding; PHD, histone binding; BROMO, acetylated Lys residues binding; FIL, actin crosslinking; NHL, protein-protein interactions; TM, transmembrane domain; ARF, domain found in ARDI (now also known as TRIM23) related to the small GTPase ARFs regulating membrane trafficking and protein sorting. (D) Representative images of TRIM knockdown cells as in (A) under basal autophagy conditions. (E) High content image analysis (TRIM siRNA screen) under basal conditions (full medium).

Encircled are scrambled siRNA controls: group on the left, pp242-induced wells; group on the bottom right, DMSO vehicle. TRIM knockdowns with GFP-LC3 puncta area >3 SD (horizontal bar) above unstimulated controls are indicated by corresponding TRIM numbers. Data, one of two experiments. Numbers in squares, TRIMs identified as positive under both basal and induced conditions. Circled numbers, positive only under basal conditions. Cells treated with TRIM63 siRNA showed signs of apoptosis and were excluded from consideration.

### TRIM5α Positively Regulates Autophagy Initiation

For detailed analysis of how TRIMs participate in autophagy, we chose to focus on TRIM5α (FIG. 2A). This was in part based on prior observations that TRIM5α may associate with the autophagy receptor p62 (O'Connor et al., 2010) albeit no connections with autophagy have been previously suggested.

FIG. 2A/A illustrates mapping of the p62/Sequestosome 1 region interacting with RhTRIM5α. Schematic (left): domain organization of p62 and deletion/point mutation constructs employed to analyze interactions with RhTRIM5α(right panel). TR, TRIM5α and TRAF6 binding region; NLS, nuclear localization signal; NES, nuclear export signal; LIR, LC3-interacting region; KIR, KEAP1-interacting region. Analysis (right): Myc-RhTRIM5α was radiolabeled with [³⁵S]methionine by in vitro translation and analyzed by GST pulldown assays with GST-p62 fusion proteins. Top, autoradiogram. Bottom, Coomassie Brilliant Blue (CBB)-stained SDS-polyacrylamide gel with GST-p62 proteins. (B) Effects of TRIM5α knockdown in HeLa cells on GFP-LC3 puncta (area/cell) under basal (DMSO control) and autophagy-inducing (pp242) conditions. HuT5a, human TRIM5α; Scr, Scrambled control. (C, D) Effect of TRIM5α knockdown on LC3-II conversion in HeLa cells upon pp242 treatment (all cells were treated with Bafilomycin A1). (E) LC3B-II/actin ratios in cells expressing GFP, GFP-HuTRIM5α or GFP-RhTRIM5α in 293T cells±befilomycin A1 (Baf); CT, control without Baf. (F) High content analysis of LC3B puncta in HeLa cells transfected with GFP or GFP-RhTRIM5α. White mask, gating for primary objects (GFP-positive cells). Pink mask, LC3B puncta. Data, means±SE, n≥3 experiments **, P<0.01*, P<0.05 (t test).

We first tested p62 and TRIM5α interaction (Figure S2A) and co-localization (Figure S2B), and mapped the TRIM5α-binding domain on p62 to the region demarcated by residues 170-256 (FIG. 2A). Next, we studied how TRIM5α affected autophagy (FIG. 2B-F). When TRIM5α was knocked down, fewer LC3 puncta were detected (FIG. 2A) and LC3-II yields (FIG. 2A/B,C) upon induction with pp242 were reduced. Conversely, overexpression of TRIM5α induced autophagy. In the latter experiments we used clones of TRIM5α from two different species, human and rhesus macaque, which show differences in certain aspects of their biological activities associated with interacting protein substrates (Stremlau et al., 2006). Overexpression of GFP-tagged TRIM5α from either source increased the abundance of LC3-II (FIG. 2D). It also increased LC3 puncta (FIG. 2A/E) relative to cells over-expressing GFP alone. In these experiments, high content analysis was used to differentiate GFP-TRIM5α-transfected or GFP-transfected cells from untransfected cells based on their green fluorescence. Using the standard bafilomycin A1 flux assay (Mizushima at al., 2010), we found that TRIM5α overexpression induced autophagy rather than blocked autophagic maturation (FIG. 2A/D). Both human TRIM5α (HuTRIM5α) and rhesus TRIM5α (RhTRIM5α) induced autophagy (FIG. 2A/D), indicating that effects on autophagy activation were independent of differences in substrate-binding domains. These findings establish TRIM5α as a regulator of autophagy induction.

Since the screen in which TRIMs, including TRIM5α, were identified as affecting autophagy, was based on mTOR inhibition with pp242, which leads to induction of autophagy via ULK1, we wondered whether TRIM5α showed any physical association with parts of this key regulatory system.

Referring to FIG. 3A, (A) Lysates from cells HeLa cells stably expressing HA-tagged Rhesus TRIM5α and transiently over-expressing either GFP-ULK1 or GFP alone were subjected to immunoprecipitation with anti-HA antisera and immunoblots probed with anti-GFP antisera. (B) Lysates from HA-RhTRIM5α-expressing cells were subjected to immunoprecipitation with either anti-HA antisera or an isotype control and immunoblots probed with antisera recognizing ULK1. (C) Confocal immunofluorescence microscopy of the localization of HA-tagged TRIM5a (rhesus; green) and endogenous ULK1 (red) in HeLa cells.

We found that HA-RhTRIM5α coimmunoprecipitated in HeLa cells with both overexpressed GFP-ULK1 (FIG. 3A/A) and endogenous ULK1 (FIG. 3A/B). ULK1 and RhTRIM5α colocalized in HeLa cells stably expressing RhTRLM5α (FIG. 3A/C). The association between TRIM5α and ULK1 is in keeping with the role of TRIM5α in induction of autophagy as first detected downstream of mTOR inhibition in the initial screen and in the follow-up experiments.

TRIM5α Interacts with Key Autophagy Regulator Beclin 1

The autophagy factor Beclin 1 is essential for autophagy induction (Liang et al., 1999; Mizushima et al., 2011). We considered whether TRIM5α might interact with Beclin 1. Endogenous Beclin 1, and its interacting co-factors AMBRA1 (Fimia at al., 2007) and ATG14L (Itakura at al., 2008; Sun et alt, 2008), co-immunoprecipitated with overexpressed rhesus TRIM5α in HeLa cells (FIG. 4A/A) whereas Beclin 1 co-immunoprecipitated endogenous TRIM5α in the rhesus cell line FRhK4 (Figure S3A), indicating that TRIM5α is in a complex with proteins involved in autophagy initiation.

Referring to FIG. 4A/A top: lysates from cells stably expressing HA-tagged Rhesus TRIM5α were subjected to immunoprecipitation with anti-HA antisera and immunoblots probed with the indicated antisera. Bottom: lysates as above were subjected to immunoprecipitation with anti-Beclin 1 antisera and immunoblots probed for HA-RhTRIM5α. (B, C) Proximity ligation assay (PLA) for direct in situ protein-protein interactions between HA-RhTRIM5α (antibody #1/Ab#1 to HA tag) and Beclin 1 of TAK-1 (antibody #2/Ab#2 to endogenous Beclin 1 or TAK1). (D) Schematic of PLA assay: for directly interacting proteins (approximating FRET distances) the distance between Ab#1 and Ab#2 allows a PCR reaction to generate fluorescent puncta (positive signal). (E) Schematic of Beclin 1 domains and interactions with indicated partners. (F) Mapping of Beclin 1 regions interacting with HA-HuTRIM5α. Lysates of 293T cells co-expressing HA-tagged TRIM5α (human) and the indicated FLAG-tagged Beclin 1 constructs were subjected to immunoprecipitation with anti-HA antisera and immunoblots probed with anti-FLAG antisera.

TRIM5α and Beclin 1 interaction was confirmed by proximity ligation assay (PLA; FIG. 4A/B,C), which reports direct protein-protein interactions in situ (FIG. 4A/D) (Soderberg at al., 2006). Positive PLA readouts of direct in situ interactions between proteins appear as fluorescent dots, the products of in situ PCR that generates a fluorescent product physically attached to antibodies against the two proteins being interrogated by PLA (FIG. 4A/B, D). Positive PLA results with Beclin 1-TRIM5α were comparable to those with proteins known (O'Connor at al., 2010; Pertel et al., 2011) to be in complexes with TRIM5α, i.e. p62 and TAB2 (Figure S3B, C), but not TAK1 (Figure S3C) that nevertheless co-localized with TRIM5α (Figure S3D).

To map Beclin 1 domains required for interactions with TRIM5α, co-immunoprecipitation experiments were carried out with human (FIG. 4A/E-F) and rhesus TRIM5α (Figure S3E). Both HuTRIM5α and RhTRIM5α bound human Beclin 1 at regions defined by residues 1-255 (encompassing the BH3 and CCD domains) and 141-450 (encompassing the CCD and ECD domains) (FIG. 4E). The CCD domain alone (residues 141-265) was insufficient for TRIM5α binding (FIG. 4E-F). Thus, TRIM5α directly interacts with Beclin 1 at two sites (FIG. 4E).

TRIM5α Affects Beclin 1 Association with its Negative Regulators Bcl-2 and TAB2

We addressed the mechanism whereby TRIM5α regulates autophagy induction. TRIM5α expression caused release of two Beclin 1 inhibitors, TAB2 (Criollo et al., 2011; Takaesu at al., 2012) and Bcl-2 (Wei et al., 2008) from Beclin 1 complexes. Beclin 1-Bcl-2 interactions were diminished when either HuTRIM5α or RhTRIM5α were overexpressed (FIG. 5A).

Referring to FIG. 5A, Bcl-2-Beclin 1 complexes assessed by co-immunoprecipitation from control cells or cells expressing HA-HuTRIM5α or HA-RhTRIM5α. (B-C) Abundance of TAB2-Beclin 1 complexes assessed by co-immunoprecipitation from 293T expressing GFP-RhTRIM5α or GFP alone. (D) PLA probing TAB2-Beclin 1 interactions in HeLa cells expressing GFP-TRIM5α or GFP alone (white mask). PLA, red dots; diffuse (GFP) or punctuate (GFP-RhTRIM5α) green fluorescence. Data, means±SE **, P<0.01 (t test).

Overexpression of GFP-RhTRIM5α dissociated TAB2 from Beclin 1 (FIG. 5A/B,C) and reduced PLA signal representing Beclin 1-TAB2 interactions, when cells identified as expressing GFP and GFP-RhTRIM5 were compared (FIG. 5A/D). Thus, TRIM5α can promote dissociation of negative regulators from Beclin 1.

### TRIM5α Induces Autophagy in a TRAF6-Dependent Manner

E3 ligases, such as TRAF6, have been implicated in control of key autophagy regulators (Shi and Kehrl, 2010). Most TRIMs, including TRIM5α, contain a RING E3 ligase domain (FIG. 1A/C). We thus tested whether the TRIM5α E3 ubiquitin ligase domain plays a role in autophagy induction. The catalytically inactive C15A mutant of RhTRIM5α (Javanbakht t al., 2005; Yamauchi et al., 2008) induced autophagy comparably to wt RhTRIM5α (FIG. 6A/A). Despite this, Ubc13, an E2 ubiquitin ligase utilized by TRIM5α (Pertel et al., 2011), was necessary for autophagy induction by TRIM5α since a knockdown of Ubc13 abrogated GFP-RhTRIM5α-induced autophagy indistinguishably from an ATG7 knockdown (FIG. 6A/B-C). This suggested that an E3 ligase other than TRIM5α was involved. An E3 ligase utilizing Ubc13 (Fukushima at al., 2007), TRAF6, induces autophagy by ubiquitination of Beclin 1 (Shi and Kehrl, 2010). TRAF6 was needed for optimal TRIM5α-induced autophagy (FIG. 6B-C). TRAF6 also co-immunoprecipitated with RhTRIM5α (FIG. 6A/D), in keeping with its role in TRIM5α-induced autophagy. In sum, TRIM5α induces autophagy in a manner independent of its own E3 ligase activity but dependent on the E3 ligase TRAF6 (Shi and Kehrl, 2010) found in complexes with TRIM5α as shown here.

TRIM5α Associates with LC3 in a p62-Dependent Manner

A positive co-localization of TRIM5α with LC3 could lend further support to TRIM5α engagement in autophagy. We tested whether TRIM5α associates with membranes positive for LC3. Confocal microscopy revealed that TRIM5α co-localized with punctuate LC3 with substantial overlap in cells treated with the autophagy inducer rapamycin (Figure S4A). Isopycnic fractionation experiments demonstrated that TRIM5α was present on intracellular membranes and co-fractionated with LC3-II, enhanced upon induction of autophagy (Figure S4B). Furthermore, TRIM5α and LC3 co-immunoprecipitated (FIG. 6A/E).

FIG. 6A/A shows high content analysis of endogenous LC3B puncta in HeLa cells expressing WT or C15A mutant GFP-RhTRIM5α. Fold induction, area per cell of LC3B puncta relative to transfection with GFP alone. (B) As in A, with HeLa cells expressing GFP-TRIM5α and subjected to the indicated siRNA knockdowns (Scr, scrambled siRNA). Data, means±SE n≥3 experiments, *, P<0.05; †, P≥0.05 (ANOVA). (C) Immunoblot analysis of Ubc13, ATG7, TRAF6, and p62 knockdowns in HeLa cells (control, non-targeting siRNA). (D) Lysates from cells stably expressing HA-tagged Rhesus TRIM5α were subjected to immunoprecipitation with anti-HA antisera and immunoblots probed with anti-TRAF6 antisera. (E,F) Assessment of interaction between GFP-LC3B and HA-tagged TRIM5α (rhesus) in control cells or cells subjected to p62 knockdown by co-immunoprecipitation. Data, means±SE; n≥3 experiments *; P<0.05 (t test).

Since p62 is known to bind to LC3, and as shown here (FIGS. 2A/A and S2) and elsewhere (O'Connor et al., 2010) to TRIM5α, we wondered whether p62 was necessary for TRIM5α association with LC3. When tested in immunoprecipitation experiments, knockdowns of p62 diminished TRIM5α and GFP-LC3 association (FIG. 6A/E-F). This is in keeping with a role for p62 in bridging 1C3 with TRIM5α.

### TRIM5α as a New Autophagic Adaptor

The above experiments establish a role for TRIM5α primarily as a regulator of autophagy induction. However, we wondered whether TRIM5α may play an additional role in autophagy by targeting a specific viral capsid protein for autophagic degradation.

FIG. 7A/A shows a schematic of rhesus TRIM5α (RhTRIM5α), emphasizing HIV-1 capsid protein (p24) binding domain and key residues (asterisks). V₁₋₄, variable regions. (B-C) Levels of intracellular p24 were determined by immunoblotting from rhesus cells (FRhK4) that had been exposed to pseudotyped virus including HIV-1 p24 for 4 h in the presence or absence of lysosomal protease inhibitors e64d and pepstatin A (e64d). (D-E) Levels of intracellular p24 were determined in FRhK4 cells that had been subjected to knockdown of autophagy factors or TRIM5α and then exposed to virus as in (B) under full media or starvation conditions for 4 h. (Scr, scrambled siRNA). (F-G) Luciferase activity of fed or starved FrhK4 cells infected with luciferase-expressing pseudotyped HIV-1 (b) or SIVmac239 (c) after depletion of indicated proteins. Data, means±SE; n≥3 experiments; *, P<0.05; †, P≥0.05 (t test).

TRIM5α contains a SPRY domain (FIG. 7A/A) which has been well established as directly binding to a protein target, the retroviral capsid protein known in human immunodeficiency virus 1 (HIV-1) as p24 or CA (p24) (Stremlau et al., 2006). We thus tested whether the HIV-p24 can be a substrate for lysosomal degradation. The experiments were carried out in FRhK4 rhesus cells using a VSVG-pseudotyped HIV-1 core viral particle. This experimental set up was chosen since it is known that the SPRY domain of rhesus TRIM5α can bind HIV-1 p24 much more efficiently than the human TRIM5α, which in turn binds HIV-1 p24 poorly and is believed to be one of the reasons for human susceptibility to HIV-1 (Stremlau et al., 2006). The experiments in FIG. 7A/B,C indicated that inhibition of lysosomal proteases protected HIV-1 p24 from degradation in rhesus cells when FRhK4 cells were infected with VSVG-pseudotyped HIV-1. We next tested whether the observed lysosomal degradation of p24 was through autophagy. The data in FIG. 7A/D,E indicated that p24 degradation was dependent on the autophagy factors Atg7, Beclin 1, p62, and TRIM5α. Induction of autophagy by starvation increased degradation of p24 in control cells but not in FRhK4 cells subjected to Atg7, Beclin 1, p62 or TRIM5α knockdowns (FIG. 7D,E). Similar results were obtained using primary Rhesus CD4 T cells (Figure S5A, B). Furthermore, ALFY/WDFY3, a potentiator of p62-dependent autophagy (Filimonenko et al., 2010), co-localized with both TRIM5α and HIV-1 p24 (Figure S6A), and a knock-down of ALFY in FRhK4 cells protected p24 from degradation, abrogating any effects of starvation (Figure S6B,C). Collectively, these data are consistent with the interpretation that p24 is a target for autophagic degradation and that this process is directed by TRIM5α in cooperation with p62 and ALFY.

Since upon viral entry TRIM5α recognizes the capsid protein (p24) only in the specific tertiary structure of the viral capsid (Stremlau et al., 2006), we considered for further study the use of a viral output assay. This was possible, since knocking down TRIM5α or autophagy factors resulted in an increase of the abundance of proviral DNA (Figure S5C) and reverse transcriptase (Figure S5D) in accordance with the results from the p24 assay described above. Having established that autophagy can lead to destruction of a portion of the incoming HIV-1 viral particles in cells expressing RhTRIM5α, we next sought to determine if this function was dependent on the specific interaction between TRIM5α and its protein target. To do this, we utilized a well-characterized feature of the RhTRIM5α SPRY domain that recognizes HIV-1 p24 but is unable to recognize the equivalent simian immunodeficiency virus (SIV) capsid protein (Stremnlau et al., 2004; Stremlau et al., 2006). A prediction based on this property of RhTRIM5α is that rhesus TRIM5α and autophagy can act upon HIV but cannot affect SIV. To test this hypothesis, we employed an assay with viral infection measured by luciferase outputs. Autophagy, induced by starvation, as in the p24 assays above, resulted in a reduced output with HIV but not with SIV (FIG. 7A/F,G). Moreover, Atg7, Beclin 1, p62 and TRIM5α were all required for optimal effects, again affecting luciferase outputs only in the case of HIV but not SIV (FIG. 7A/F,G). This establishes that mobilization of the viral target for degradation by the autophagic apparatus directly correlates with the sequence-specific binding specificity of TRIM5α.

### DISCUSSION

This study identifies TRIM family members as regulators of autophagy. Using one specific TRIM, TRIM5α, we uncovered two mechanisms of how it acts in autophagy. Firstly, TRIM5α interacts with two central regulators of autophagy, ULK1 and Beclin 1, and promotes autophagy initiation by liberating Beclin 1 from its negative regulators TAB2 and Bcl-2. Secondly, TRIM5α acts as a receptor by directly recognizing its cognate target destined for autophagic degradation. TRIM5α cooperates with other components of the autophagic apparatus, including binding to another autophagy receptor p62 and to the adaptor ALFY, which bridge autophagic cargo with LC3-positive membranes (Isakson et al., 2013; Johansen and Lamark, 2011) and, at least in the case of p62, play additional roles in signaling (Komatsu et al, 2010; Mathew et al., 2009; Moscat and Diaz-Meco, 2009) and several aspects of autophagy (Isakson et al., 2013; Johansen and Lamark, 2011). Based on these features, TRIM5α links the recognition of the target, induction of autophagy, and assembly of autophagic membranes.

Like TRIM5α, TRIM13, TRIM21 (Ro52), and TRIM50 all interact with the autophagy adaptor p62/sequestosome 1 (Fusco et al., 2012; Kim and Ozato, 2009; O'Connor et al., 2010; Tomar et al., 2012). Furthermore, both TRIM30a and TRIM21 target cytosolic proteins for lysosomal degradation (Niida et al., 2010; Shi at al., 2008), probably through autophagy. TRIM5α is known to directly recognize capsid sequences via its SPRY domain (Stremlau et al., 2004; Stremlau at al., 2006) and should be considered as an example of high fidelity selective autophagy in mammalian cells. Most TRIMs contain SPRY or other types of C-terminal domains (FIG. 1B) with the potential to recognize diverse protein targets or other molecular patterns (Kawai and Akira, 2011). A list of such domains in TRIMs, besides SPRY, paired with known types of targets include: COS—microtubule binding; FN3—DNA or heparin binding; PHD—histone binding; BROMO—acetylated Lys residues binding; FIL—actin crosslinking; and NHL—protein interactions. Thus, we propose that TRIM proteins, as a group, may comprise a new class of broad-repertoire autophagic adaptors. In principle, these adaptors may directly recognize their cognate targets without a need for ubiquitin tagging. This may engender an exclusive recognition specificity as in the case of TRIM5α, in a process that we dub here as boutique autophagy.

The above principle contrasts with but does not contradict the well-established model that mammalian cells use target ubiquitination as a major tag for recognition of autophagic cargo (Shaid et al., 2013). The TRIM5α action is more akin to selective autophagy in yeast where this process is independent of ubiquitin tags including the Cvt pathway (Lynch-Day and Klionsky, 2010), mitophagy (Kanki at al., 2009; Okamoto at al., 2009), and pexophagy (Farre at al., 2008). There are also emerging examples in metazoans whereby selective autophagy occurs independently of ubiquitin (Johansen and Lamark, 2011). This includes organisms from C. elegans (Zhang et al., 2009) to mammals (Gal at al., 2009; Orvedahl at al., 2010). Furthermore, the p62-dependent autophagic protection against Sindbis virus (Orvedahl at al., 2010) and autophagic removal of mutant superoxide dismutase 1 associated with amyotrophic lateral sclerosis (Gal et al., 2009) appear to rely on ubiquitin-independent functions of p62 in autophagy, which may mirror p62's contribution to TRIM5α action revealed hero. It has also been proposed that other signals on target membranes such as diacylglycerol (Shahnazari et al., 2010) or phospholipids (Orvedahl et al., 2011) including mitochondria-specific cardiolipin (Singh at al., 2010), or β-glycoside-galectin complexes on ruptured endomembranes (Thurston et al., 2012) may serve to guide autophagy receptors to its targets. Thus, the findings that TRIM5α and potentially other TRIMs, may provide ubiquitin tag-independent recognition may not be an exception. However, TRIMs offer, at least in the example of TRIM5α, high fidelity selectivity by direct binding their targets via cargo-recognition domains such as SPRY. This has the potential to expand the autophagic target recognition mechanisms both in breadth and in terms of specificity and exclusivity that a generic tagging with ubiquitin lacks.

The observation that C15A mutation, which abrogates F3 ligase activity of TRIM5α (Javanbakht al., 2005; Yamauchi et al., 2008), does not preclude TRIM5α action in autophagy is in keeping with and complements the previously established notion that ubiquitin ligase activity of TRIM5α is not needed for its action against the viral capsid protein p24 (Diaz-Griffero et al., 2006; Javanbakht et al., 2005). Similarly, The E3 ligase domains present in a number of other newly identified selective autophagy adaptors are not required, as in the case of c-Cb1-dependent delivery of src (Sandilands et al., 2012) or SMURF1 targeting of mitochondria (Orvedahl et al., 2011) for autophagy. This does not contradict the established processes (Kirkin et al., 2009b; Shaid et al., 2013) of autophagic targeting in mammalian cells that are dependent on ubiquitin tags and their recognition via ubiquitin-binding autophagy receptors p62 (Bjorkoy et al., 2005; Komatsu et al., 2007; Pankiv et al., 2007), NBR1 (Kirkin et al., 2009a), NDPS2 (Thurston et al., 2009), and optineurin (Wild et al., 2011). These classical receptors require independent E3 ligase entities and activities to mark the autophagic targets with poly-ubiquitin chains (Huett et al., 2012; Yoshii et al., 2011; Youle and Narendra, 2011) since they do not possess their own E3 ligase activities. The F3 ligase domains found in adaptors such as c-Cb1 (Sandilands et al., 2012), SMURF1 (Orvedahl et al., 2011), and TRIMs, may regulate stability of these proteins, as shown for TRIM5α (Diaz-Griffero at al., 2006).

The association with TRIM5α and functional participation of p62 and ALFY in the context of TRIM5α can be best explained in the context of p62 being a known binding partner for LC3 (Ichimura et al., 2008; Noda et al., 2008; Pankiv et al., 2007), whereas ALFY has been proposed (Isakson et al., 2013) to act as a mammalian equivalent of the yeast protein Atg11 interacting with receptors Atg119 (Lynch-Day and Klionsky, 2010), Atg30 (Farre et al., 2008), and Atg32 (Kanki et al., 2009; Okamoto at al., 2009), conducting several forms of selective autophagy in yeast (the Cvt pathway, pexophagy, and mitophagy). Thus, a complex between TRIM5α, p62, and ALFY may ensure high fidelity cargo recognition via TRIM5α, as shown here, binding to LC3 via p62 (Ichimura et al., 2008; Noda et al., 2008; Pankiv et al., 2007), and association of ALFY with phosphatidylinositol 3-phosphate containing endomembranes (Simonsen et al., 2004) believed to be the precursors to autophagosomes (Axe et al., 2008). We cannot exclude an intriguing possibility that p62 may, as a back-up system, secondarily recognize ubiquitinated cargo that escapes recognition by the TRIM5α SPRY domain.

Importantly, TRIMs may not be just autophagic adaptors but, as shown for TRIM5α, may carry out activation of autophagy via Beclin 1 in addition to cargo binding. Thus, TRIM5α embodies in one core entity two essential aspects of selective autophagy-recognition of the cargo and initiation of autophagy. A reminiscent role in controlling the rate of autophagy may be seen in the Atg11-Atg19 system, since increased expression of Atg11 can lead to increased formation of Cvt vesicles in yeast (Lynch-Day and Klionsky, 2010). Thus the Cvt system and TRIM5α share the capacity to recognize targets and drive their elimination or processing.

The role of TRIM5α as a regulator of autophagy can be modeled on its connections to TRAF6. Inactivation of the TRIM5α RING domain (Javanbakht et al., 2005; Yamauchi et al., 2008) did not abrogate its ability to act in autophagy but TRAF6 was key to autophagy induction by TRIM5α. TRIM5α co-immunoprecipitates with TRAF6; this interaction may be aided by p62 that associates with TRAF6 (Moscat and Diaz-Meco, 2009; Sanz et al., 2000). Actually, both TRIM5□ and TRAF6 bind to the same general region of p62 (TR, FIG. 2A). As demonstrated here, TRIM5α displaces Bcl-2 and TAB2 from Beclin 1. This may occur by competition or through the action of TRAF6 as an E3 ubiquitin ligase. Our data indicating that TRAF6 and E2 enzyme Ubc13 are required for induction of autophagy by TRIM5α favor the latter possibility at least in the case of Bcl-2 displacement from Beclin 1, which has been previously shown to occur upon TRAF6-dependent polyubiquitination of Beclin 1 (Shi and Kehrl, 2010). TAB2 may also be under the control of ubiquitin chains, as TAB2 displacement from Beclin 1 has been described during induction of autophagy by physiological stimuli such as starvation (Criollo et al., 2011).

Additionally, TAB2 has been shown to be a substrate for ULK1 phosphorylation (Takaesu et al., 2012), and thus TRIM5α association with ULK1 may further explain the observed TAB2 dissociation from Beclin 1. While our work was in preparation, a recent report (Nazio et al., 2013) has implicated TRAF6 in acting upon ULK1 via AMBRA1, an ancillary factor in autophagy initiation (Fimia et al., 2007). We have detected AMBRA1 in complexes with TRIM5α, and thus the TRAF6 action in the context of TRIM5α initiation of autophagy may extend to ULK1. Since ULK1 is the key target for regulation by mTOR and our screen was carried out with an inhibitor of mTOR, pp242, the latter may help explain why TRIM5α is required for optimal pp242-induction of autophagy. Potentially, the above relationships may extend to other members of the TRIM family showing effects on autophagy.

In conclusion, our study reports the recognition of a global control of autophagy in mammalian cells by TRIM family members. Our screen reveals that, in addition to cytokine responses (Kawai and Akira, 2011; Ozato et al., 2008; Pertel et al., 2011; Versteeg et al., 2013), TRIMs as a family use autophagy as one of their major biological outputs. In support of this, TRIMs 25, 29, 33 and 69 are separately found in lists of genome-wide autophagy screens (Behrends t al., 2010; Lipinski at al, 2010; McKnight et al., 2012). We furthermore have defined two roles whereby one of the TRIM family members, TRIM5α, acts both to promote autophagy induction and as a ubiquitin-tag independent adaptor for a specific autophagic cargo: retroviral capsid. TRIMs have roles in antiviral defense (Jefferies et al., 2011; Stremlau et al., 2004) and it might be of interest to test whether TRIMs do this through autophagy. TRIMs furthermore influence inflammation and immune responses (Versteeg et al., 2013), development (Cavalieri at al., 2011) and chromatin remodeling and transcriptional control (Chen t al., 2012). Accordingly, several human diseases including Crohn's disease, familial Mediterranean fever, and various cancers have been linked to TRIM family members (Hatakeyama, 2011; Jefferies at al., 2011; Kawai and Akira, 2011). Our study opens possibilities that the roles of TRIM proteins in these diverse processes and diseases are through autophagy and invites explorations of these novel connections.

### Materials and Methods

### Cells and Viruses

HeLa, 293T, and FRhK4 cells (from ATCC) were cultured in DMEM containing 10% fetal calf serum. Primary rhesus CD4+ T cells were enriched by depletion of CD8+ cells from peripheral blood-derived non-adherent lymphocytes, activated with concanavalin A, and maintained in RPMI supplemented with 1% human serum, 10% fetal calf serum, 50 μM β-mercaptoethanol, and human 10 ng mL⁻¹ IL-2. HeLa cells stably expressing mRFP-GFP-LC3B (from D. Rubinsztein, Cambridge University) were used for TRIM5α siRNA screen and maintained in complete DMEM containing 500 μg mL⁻¹ G418 while HeLa cells stably expressing HA-RhTRIM5α (from J. Sodroski, Harvard University) were maintained in media containing 1 μg mL⁻¹ of puromycin as a positive selection agent. Single cycle HIV-1 or SIV_{mac239} viruses were generated by co-transfection of plasmids encoding the NL43 or SIV_{mac239} clones lacking the env gene and VSV-G protein into 293T cells.

Plasmid, siRNA, and Transfection

HA- and GFP-tagged TRIM5α expression plasmids (from J. Sodroski) have been described previously (Song et al., 2005; Stremlau et al., 2004), as have those for FLAG-Beclin 1 (Shoji-Kawata et al., 2013). The GFP-RhTRIM5α_{C15A} mutant was generated from GFP-RhTRIM5α expression clone by site-directed mutagenesis and mutation confirmed by sequencing. RhTRIM5α was amplified from HA-RhTRIM5α plasmid using Phusion® High-Fidelity DNA Polymerase (New England Biolabs) with primers containing the BP cloning site and recombined into the pDONR221 vector. pDestMyc-RhTRIM5α expression plasmid was made from pDONR221-RhTRIM5α plasmid using the LR reaction. Gateway BP and LR reactions were performed as per the Gateway manual (Invitrogen). All siRNAs were from Dharmacon. With the exception of the siRNA transfections for the TRIM screens (with siRNA printed into the 96 well plates), all siRNA were delivered to cells by nucleoporation (Amaxa) of 1.5 μg of siRNA. Plasmid transfections were performed by either CaPO₄ or nucleoporation (Amaxa).

### Infection and Treatments

Cells were exposed to virus at 4° C. for 1 hour to allow binding but not entry. Unbound virus was removed by washing and bound virus was allowed to infect cells under basal or induced autophagy conditions (starvation or rapamycin) at 37C for 4 h. Samples were prepared for analysis of p24, reverse transcriptase, or proviral DNA. For assays with luciferase, siRNA-treated and infected (as above) cells were maintained in full media for 48 h following the 4 h infection period. HIV-1 RT was determined according to the manufacturer's protocol (Enz Chek, Invitrogen). HIV-1 proviral DNA was quantified as previously described (Campbell at al., 2004). Working concentrations for inhibitors were as follows: pp242, 10 μg ml⁻¹; e64d, 10 μg ml⁻¹; pepstatin A, 10 μg ml⁻¹; Rapamycin, 50 μg ml⁻¹; MG132, 500 ng ml⁻¹; Bafilomycin A1, 60 ng ml⁻¹.

### TRIM Family Screen

HeLa cells stably expressing mRFP-GFP-LC3B were cultured in 96-well plates containing siRNAs against 67 human TRIMs and transfection reagent (Dharmacon). 48 h after plating, cells were treated as indicated with pp242 for 2 h, fixed, and stained with Hoechst 33342. High content imaging analysis was performed using a Cellomics HCS scanner and iDEV software (Thermo). Automated image collection of >500 cells (distributed over 49 or fewer fields per well per siRNA knockdown per plate) were machine-analyzed using preset scanning parameters and object mask definition (iDEV software). Cell were identified by the program routine based on nuclear staining and cell outlines defined by background staining of the cytoplasm, and the mean per cell total area of GFP puncta or number of GFP puncta per cell were reported. Autophagy induction with pp242 resulted in a 17-fold induction of GFP-LC3B puncta area and a Z′ value (robustness of the assay) of 0.52. TRIMs whose mean total area of GFP-LC3 per cell in three separate siRNA screen experiments (autophagy induced with pp242) differed by >3 standard deviation above and below the mean of pp242-treated controls were reported as hits. For basal autophagy, two separate siRNA screens were carried out with the same cutoff (>3 SD above the mean of unstimulated controls) for hits. When results were expressed as puncta area per cell, the units corresponded to μm²/cell.

### High Content Analysis of Puncta in Subpopulations of Transfected Cells

HeLa cells were transfected with GFP or GFP-RhTRIM5α plasmids with or without siRNA, and cultured in full media for 48 h. Cells were then stained to detect LC3, GFP, and nuclei. High content imaging and analysis was performed using a Cellomics HCS scanner and iDEV software (Thermo)>200 cells were analyzed per treatment in quadruplicate per experiment. Cell outlines were automatically determined based on background nuclear staining, and the mean total area of punctuate LC3 per cell was determined within the sub-population of cells that were successfully transfected as determined by having above background GFP fluorescence.

### Proximity Ligation Assay

Proximity ligation assay (PLA) was performed as described (Pilli et al., 2012). PLA reports direct in situ interactions between proteins revealed as fluorescent dots, the products of in situ PCR that generates a fluorescent product physically attached to antibodies against the two proteins being interrogated by PLA. When the antibodies bound to proteins in situ are <16 nm apart (FRET distance) positive PCR signals emerge that are revealed by imaging as fluorescent puncta. PLA results were reported as average number of red puncta per cell or total intensity of the PLA signal (sum of all puncta intensity) within green-fluorescent (transfected) cells using ImageJ software.

### Immunoblotting, Immunolabeling for Microscopy, Co-Immunoprecipitation, Subcellular Fractionation, and GST Pulldown Experiments

Immunoprecipitation, immunoblots, immunofluorescent labeling, and subcellular organellar fractionation were as described (Kyei et al., 2009). Antibodies used were: AMBRA1 (Novus), ATG7 (Santa Cruz), ATG14L (MBL), Beclin 1 (Novus and Santa Cruz), Flag (Sigma), HA (Sigma and Roche), p62 (Abcam), TAB2 (Santa Cruz), TAK1 (Abcam), TRAF6 (Abcam), TRIM5α (Abcam), UBC13 (Abcam), ULK1 (Sigma). All other antibodies were as described (Kyei at al., 2009). GST and GST-tagged proteins were expressed in Escherichia coli BL21(DE3) or SoluBL21 (Amsbio). GST and GST-fusion proteins were purified and immobilized on glutathione-coupled sepharose beads (Amersham Bioscience, Glutathione-sepharose 4 Fast Flow) and pulldown assays with in vitro translated [³⁵S]-labeled proteins were done as described previously (Pankiv et al., 2007). The [³⁵S] labeled proteins were produced using the TNT T7 Quick Coupled Transcription/Translation System (Promega) in the presence of [³⁵S] L-methionine. The proteins were eluted from washed beads by boiling for 5 min in SDS-PAGE gel loading buffer, separated by SDS-PAGE, and radiolabeled proteins detected in a Fujifilm bioimaging analyzer BAS-5000 (Fuji).

### Statistical Analyses

Either a two-tailed Student's t test or ANOVA were used. Pearson's colocalization coefficient (R_{f}=Σ[(S₁ᵢ−S_{1avg})×(S₂ᵢ−S_{2avg})]/[E(S₁ᵢ−S_{1avg}²×Σ(S₂ᵢ−S_{2avg})²]^{1/2} (R_{f} values range: ≥−1 R_{f}≤+1) was calculated using SLIDEBOOK 5.0 (Intelligent Imaging Innovations).

### REFERENCES FOR EXAMPLE 3

Axe, E. L., Walker, S. A., Manifava, M., Chandra, P., Roderick, H. L., Habermann, A., Griffiths, G., and Ktistakis, N. T. (2008). Autophagosome formation from membrane compartments enriched in phosphatidylinositol 3-phosphate and dynamically connected to the endoplasmic reticulum. J Cell Biol 182, 685-701.
Behrends, C., Sowa, M. E., Gygi, S. P., and Harper, J. W. (2010). Network organization of the human autophagy system. Nature 466, 68-76.
Bjorkoy, G., Lamark, T., Brech, A., Outzen, H., Perander, M., Overvatn, A., Stenmark, H., and Johansen, T. (2005). p62/SQSTM1 forms protein aggregates degraded by autophagy and has a protective effect on huntingtin-induced cell death. J Cell Biol 171, 603-614.
Campbell, E. M., Nunez, R., and Hope, T. J. (2004). Disruption of the actin cytoskeleton can complement the ability of Nef to enhance human immunodeficiency virus type I infectivity. Journal of virology 78, 5745-5755.
Cavalieri, V., Guarcello, R., and Spinelli, G. (2011). Specific expression of a TRIM-containing factor in ectoderm cells affects the skeletal morphogenetic program of the sea urchin embryo. Development 138, 4279-4290.
Chen, L., Chen, D. T., Kurtyka, C., Rawal, B., Fulp, W. J., Haura, E. B., and Cress, W. D. (2012). Tripartite motif containing 28 (Trim28) can regulate cell proliferation by bridging HDAC1/E2F interactions. The Journal of biological chemistry 287, 40106-40118.
Criollo, A., Niso-Santano, M., Malik, S. A., Michaud, M., Morselli, E., Marino, G., Lachkar, S., Arkhipenko, A. V., Harper, F., Pierron, G, et al. (2011). Inhibition of autophagy by TAB2 and TAB3. The EMBO journal 30, 4908-4920.
Diaz-Griffero, F., Li, X., Javanbakht, H., Song, B., Welikala, S., Stremlau, M., and Sodroski, J. (2006). Rapid turnover and polyubiquitylation of the retroviral restriction factor TRIM5. Virology 349, 300-315.
Farre, J. C., Manjithaya, R., Mathewson, R. D., and Subramani, S. (2008). PpAtg30 tags peroxisomes for turnover by selective autophagy. Dev Cell 14, 365-376.
Filimonenko, M., Isakson, P., Finley, K. D., Anderson, M., Jeong. H., Melia, T. J., Bartlett, B. J., Myers, K. M., Birkeland, H. C., Lamark, T., et al. (2010). The selective macroautophagic degradation of aggregated proteins requires the PI3P-binding protein Alfy. Mol Cell 38.265-279.
Fimia, G. M., Stoykova, A., Romagnoli, A., Giunta, L., Di Bartolomeo, S., Nardacci, R., Corazzari, M., Fuoco, C., Ucar, A., Schwartz, P., et al. (2007). Ambra1 regulates autophagy and development of the nervous system. Nature 447, 1121-1125.
Fukushima, T., Matsuzawa, S., Kress, C. L., Bruey, J. M., Krajewska, M., Lefebvre, S., Zapata, J. M., Ronai, Z., and Reed, J. C. (2007). Ubiquitin-conjugating enzyme Ubc13 is a critical component of TNF receptor-associated factor (TRAF)-mediated inflammatory responses. Proc Natl Acad Sci USA 104, 6371-6376.
Fusco, C., Micale, L., Egorov, M., Monti, M., D'Addetta, E. V., Augello, B., Cozzolino, F., Calcagni, A., Fontana, A., Polishchuk, R. S., et al. (2012). The E3-ubiquitin ligase TRIM50 interacts with HDAC6 and p62, and promotes the sequestration and clearance of ubiquitinated proteins into the aggresome. PloS one 7, e40440.
Gal, J., Strom, A. L., Kwintor, D. M., Kilty, R., Zhang, J., Shi, P., Fu, W., Wooten, M. W., and Zhu, H. (2009). Sequestosome 1/p62 links familial ALS mutant SOD1 to LC3 via an ubiquitin-independent mechanism. J Neurochem 111, 1062-1073.
Hamasaki, M., Furuta, N., Matsuda, A., Nezu, A., Yamamoto, A., Fujita, N., Oomori, H., Noda, T., Haraguchi, T., Hiraoka, Y, et al. (2013). Autophagosomes form at ER-mitochondria contact sites. Nature.
Hatakeyama, S. (2011). TRIM proteins and cancer. Nat Rev Cancer 11, 792-804.
Huett, A., Heath, R. J., Begun, J., Sassi, S. O., Baxt, L. A., Vyas, J. M., Goldberg, M. B., and Xavier, R. J. (2012). The LRR and RING Domain Protein LRSAM1 Is an E3 Ligase Crucial for Ubiquitin-Dependent Autophagy of Intracellular Salmonella Typhimurium. Cell host & microbe 12, 778-790.
Ichimura, Y., Kumanomidou, T., Sou, Y. S., Mizushima, T., Ezaki, J., Ueno, T., Kominami, E., Yamane, T., Tanaka, K., and Komatsu. M. (2008). Structural Basis for Sorting Mechanism of p62 in Selective Autophagy. The Journal of biological chemistry 283, 22847-22857.
Isakson, P., Holland, P., and Simonsen, A. (2013). The role of ALFY in selective autophagy. Cell death and differentiation 20, 12-20.
Itakura, E., Kishi, C., Inone, K., and Mizushima, N. (2008). Beclin 1 forms two distinct phosphatidylinositol 3-kinase complexes with mammalian Atg14 and UVRAG. Mol Biol Cell 19, 5360-5372.
Itakura, E., Kishi-Itakura, C., and Mizushima, N. (2012). The hairpin-type tail-anchored SNARE syntaxin 17 targets to autophagosomes for fusion with endosomes/lysosomes. Cell 151, 1256-1269.
Javanbakht, H., Diaz-Griffero, F., Stremlau, M., Si, Z., and Sodroski, J. (2005). The contribution of RING and B-box 2 domains to retroviral restriction mediated by monkey TRIM5alpha. The Journal of biological chemistry 280, 26933-26940.
Jefferies, C., Wynne, C., and Higgs, R. (2011). Antiviral TRIMs: friend or foe in autoimmune and autoinflammatory disease? Nat Rev Immunol 11, 617-625.
Johansen, T., and Lamark, T. (2011). Selective autophagy mediated by autophagic adapter proteins. Autophagy 7, 279-296.
Kabeya, Y., Mizushima, N., Ueno, T., Yamamoto, A., Kirisako, T., Noda, T., Kominami, E., Ohsumi, Y., and Yoshimori, T. (2000). LC3, a mammalian homologue of yeast Apg8p, is localized in autophagosome membranes after processing. Embo J 19, 5720-5728.
Kanki, T., Wang, K., Cao, Y., Baba, M., and Klionsky, D. J. (2009). Atg32 is a mitochondrial protein that confers selectivity during mitophagy. Dev Cell 17, 98-109.
Kawai, T., and Akira, S. (2011). Regulation of innate immune signalling pathways by the tripartite motif (TRIM) family proteins. EMBO molecular medicine 3, 513-527.
Kim, J. Y., and Ozato, K. (2009). The sequestosome 1/p62 attenuates cytokine gene expression in activated macrophages by inhibiting IFN regulatory factor 8 and TNF receptor-associated factor 6/NF-kappaB activity. Journal of immunology 182, 2131-2140.
Kirkin, V., Lamark, T., Sou, Y. S., Bjorkoy, G., Nunn, J. L., Bruun, J. A., Shvets, E., McEwan, D. G., Clausen, T. H., Wild, P, et al. (2009a). A role for NBR1 in autophagosomal degradation of ubiquitinated substrates. Mol Cell 33, 505-516.
Kirkin, V., McEwan, D. G., Novak, I., and Dikic, I. (2009b). A role for ubiquitin in selective autophagy. Molecular cell 34, 259-269.
Komatsu, M., Kurokawa, H., Waguri, S., Taguchi, K., Kobayashi, A., Ichimura, Y., Sou, Y. S., Ueno, I., Sakamoto, A., Tong, K. I., et al. (2010). The selective autophagy substrate p62 activates the stress responsive transcription factor Nrf2 through inactivation of Keap1. Nat Cell Biol 12, 213-223.
Komatsu, M., Waguri, S., Koike, M., Sou, Y. S., Ueno, T., Hara, T., Mizushima, N., Iwata, J., Ezaki, J., Murata, S., et al. (2007). Homeostatic levels of p62 control cytoplasmic inclusion body formation in autophagy-deficient mice. Cell 131, 1149-1163.
Kyei, G. B., Dinkins, C., Davis, A. S., Roberts, E., Singh, S. B., Dong, C., Wu, L., Kominami, E., Ueno, T., Yamamoto, A., et al. (2009). Autophagy pathway intersects with HIV-1 biosynthesis and regulates viral yields in macrophages. J Cell Biol 186, 255-268.
Li, S., and Randow, F. (2013). A crystal structure of an autophagy receptor with a ligand reveals how Salmonella is
specifically targeted for degradation. Science Signaling INn press.
Liang, X. H., Jackson, S., Seaman, M., Brown, K., Kempkes, B., Hibshoosh, H., and Levine, B. (1999). Induction of autophagy and inhibition of tumorigenesis by beclin 1. Nature 402, 672-676.
Lipinski, M. M., Zheng, B., Lu, T., Yan, Z., Py, B. F., Ng. A., Xavier, R. J., Li, C., Yankner, B. A., Scherzer, C. R., et al. (2010). Genome-wide analysis reveals mechanisms modulating autophagy in normal brain aging and in Alzheimer's disease. Proc Natl Acad Sci USA 107, 14164-14169.
Lynch-Day, M. A., and Klionsky, D. J., (2010). The Cvt pathway as a model for selective autophagy. FEBS Lett 584, 1359-1366.
Mathew, R., Karp, C. M., Beaudoin, B., Vuong, N., Chen, G., Chen, H. Y., Bray, K., Reddy, A., Bhanot, G., Gelinas, C, et al. (2009). Autophagy suppresses tumorigenesis through elimination of p62. Cell 137, 1062-1075.
McKnight, N. C., Jefferies, H. B., Alemu, E. A., Saunders, R. E., Howell, M., Johansen, T., and Tooze, S. A. (2012). Genome-wide siRNA screen reveals amino acid starvation-induced autophagy requires SCOC and WAC. EMBO J 31, 1931-1946.
Mizushima, N., Yoshimori, T., and Levine, B. (2010). Methods in mammalian autophagy research. Cell 140, 313-326.
Mizushima, N., Yoshimori, T., and Ohsumi, Y. (2011). The role of atg proteins in autophagosome formation. Annual review of cell and developmental biology 27, 107-132.
Moscat, J., and Diaz-Meco, M. T. (2009). p62 at the crossroads of autophagy, apoptosis, and cancer. Cell 137, 1001-1004.
Nazio, F., Strappazzon, F., Antonioli, M., Bielli, P., Cianfanelli, V., Bordi, M., Gretzmeier, C., Dengjel, J., Piacentini, M., Fimia, G. M, et al. (2013). mTOR inhibits autophagy by controlling ULK1 ubiquitylation, self-association and function through AMBRA1 and TRAF6. Nature cell biology 15, 406-416.
Niida, M., Tanaka, M., and Kamitani, T. (2010). Downregulation of active IKK beta by Ro52-mediated autophagy. Molecular immunology 47, 2378-2387.
Noda, N. N., Kumeta, H., Nakatogawa, H., Satoo, K., Adachi, W., Ishii, J., Fujioka, Y., Ohsumi, Y., and Inagaki, F. (2008). Structural basis of target recognition by Atg5/LC3 during selective autophagy. Genes Cells 13, 1211-1218.
O'Connor, C., Pertel, T., Gray, S., Robia, S. L., Bakowska, J. C., Luban, J., and Campbell, E. M. (2010). p62/sequestosome-1 associates with and sustains the expression of retroviral restriction factor TRIM5alpha. Journal of virology 84, 5997-6006.
Okamoto, K., Kondo-Okamoto, N., and Ohsumi, Y. (2009). Mitochondria-anchored receptor Atg32 mediates degradation of mitochondria via selective autophagy. Dev Cell 17, 87-97.
Orvedahl, A., Macpherson, S., Sumpter, R., Jr., Talloczy, Z., Zou, Z., and Levine, B. (2010). Autophagy Protects against Sindbis Virus Infection of the Central Nervous System. Cell Host Microbe 7, 115-127.
Orvedahl, A., Sumpter, R., Jr., Xiao, G., Ng, A., Zou, Z., Tang. Y., Narimatsu, M., Gilpin, C., Sun, Q., Roth, M., et al. (2011). Image-based genome-wide siRNA screen identifies selective autophagy factors. Nature 480, 113-117.
Ozato, K., Shin, D. M., Chang, T. H., and Morse, H. C., 3rd (2008). TRIM family proteins and their emerging roles in innate immunity. Nat Rev Immunol 8, 849-860.
Pankiv, S., Clausen, T. H., Lamark, T., Brech, A., Bruun, J. A., Outzen, H., Overvatn, A., Bjorkoy, G., and Johansen, T. (2007). p62/SQSTM1 binds directly to Atg8/LC3 to facilitate degradation of ubiquitinated protein aggregates by autophagy. The Journal of biological chemistry 282, 24131-24145.
Perrin, A. J., Jiang, X., Birmingham, C. L., So, N. S., and Brumell, J. H. (2004). Recognition of bacteria in the cytosol of Mammalian cells by the ubiquitin system. Curr Biol 14, 806-811.
Pertel, T., Hausmann, S., Morger, D., Zuger, S., Guerra, J., Lascano, J., Reinhard, C., Santoni, F. A., Uchil, P. D., Chatel, L., et al. (2011). TRIM5 is an innate immune sensor for the retrovirus capsid lattice. Nature 472, 361-365.
Pilli, M., Arko-Mensah, J., Ponpuak, M., Roberts, B., Master, S., Mandell. M. A., Dupont, N., Ornatowski, W., Jiang, S., Bradfute, S. B, et al. (2012). TBK-1 Promotes Autophagy-Mediated Antimicrobial Defense by Controlling Autophagosome Maturation. Immunity 37, 223-234.
Reymond, A., Meroni, G., Fantozzi, A., Merla, G., Cairo, S., Luzi, L., Riganelli, D., Zanaria, E., Messali, S., Cainarca, S., et al. (2001a). The tripartite motif family identifies cell compartments. The EMBO journal 20, 2140-2151.
Reymond, A., Meroni, G., Fantozzi, A., Merla, G., Cairo, S, Luzi, L., Riganelli, D., Zanaria, E., Messali, S., Cainarca, S., et al. (2001b). The tripartite motif family identifies cell compartments. EMBO J 20, 2140-2151.
Saitoh, T., and Akira, S. (2010). Regulation of innate immune responses by autophagy-related proteins. J Cell Biol 189, 925-935.
Sandilands, E., Serrels, B., McEwan, D. G., Morton, J. P, Macagno, J. P., McLeod, K., Stevens, C., Brunton, V. G., Langdon, W. Y., Vidal, M., et al. (2012). Autophagic targeting of Src promotes cancer cell survival following reduced FAK signalling. Nature cell biology 14, 51-60.
Sanz, L., Diaz-Meco, M. T., Nakano, H., and Moscat, J. (2000). The atypical PKC-interacting protein p62 channels NF-kappaB activation by the IL-1-TRAF6 pathway. The EMBO journal 19, 1576-1586.
Settembre, C., Di Malta, C., Polito, V. A., Garcia Arencibia, M., Vetrini, F., Erdin, S., Erdin, S. U., Huynh, T., Medina, D., Colella, P, et al. (2011). TFEB links autophagy to lysosomal biogenesis. Science 332, 1429-1433.
Shahnazari, S., Yen, W. L., Birmingham, C. L., Shiu, J., Namolovan, A., Zheng. Y. T., Nakayama, K., Klionsky, D. J., and Brumell, J. H. (2010). A diacylglycerol-dependent signaling pathway contributes to regulation of antibacterial autophagy. Cell host & microbe 8, 137-146.
Shaid, S., Brandts, C. H., Serve, H., and Dikic, I. (2013). Ubiquitination and selective autophagy. Cell death and differentiation 20, 21-30.
Shi, C. S., and Kehrl, J. H. (2010). TRAF6 and A20 regulate lysine 63-linked ubiquitination of Beclin-1 to control TLR4-induced autophagy. Sci Signal 3, ra42.
Shi, M., Deng, W., Bi, E., Mao, K., Ji, Y., Lin, G., Wu, X., Tao, Z., Li, Z., Cai, X., et al. (2008). TRIM30 alpha negatively regulates TLR-mediated NF-kappa B activation by targeting TAB2 and TAB3 for degradation. Nat Immunol 9, 369-377.
Shoji-Kawata, S., Sumpter, R., Leveno, M., Campbell, G. R., Zou, Z., Kinch, L., Wilkins, A. D., Sun, Q., Pallauf, K., MacDuff, D., at al. (2013). Identification of a candidate therapeutic autophagy-inducing peptide. Nature 494, 201-206.
Simonsen, A., Birkeland, H. C., Gillooly, D. J., Mizushima, N., Kuma, A., Yoshimori, T., Slagsvold, T., Brech, A., and Stenmark, H. (2004). Alfy, a novel FYVE-domain-containing protein associated with protein granules and autophagic membranes. J Cell Sci 117, 4239-4251.
Singh, S. B., Omatowski, W., Vergne, I., Naylor, J., Delgado, M., Roberts, E., Ponpuak, M., Master, S., Pilli, M., White, E., et al. (2010). Human IRGM regulates autophagy and cell-autonomous immunity functions through mitochondria. Nat Cell Biol 12, 1154-1165.
Soderberg, O., Gullberg, M., Jarvius, M., Ridderstrale, K., Leuchowius, K. J., Jarvius, J., Wester, K., Hydbring, P., Bahram, F., Larsson, L. G., et al. (2006). Direct observation of individual endogenous protein complexes in situ by proximity ligation. Nature methods 3, 995-1000.
Song, B., Diaz-Griffero, F., Park, D. H., Rogers, T., Stremlau, M., and Sodroski, J. (2005). TRIM5alpha association with cytoplasmic bodies is not required for antiretroviral activity. Virology 343, 201-211.
Stremlau, M., Owens, C. M., Perron, M. J., Kiessling, M., Autissier, P., and Sodroski, J. (2004). The cytoplasmic body component TRIM5alpha restricts HIV-1 infection in Old World monkeys. Nature 427, 848-853.
Stremlau, M., Perron, M., Lee, M., Li, Y., Song, B., Javanbakht, H., Diaz-Griffero, F., Anderson, D. J., Sundquist, W. I., and Sodroski, J. (2006). Specific recognition and accelerated uncoating of retroviral capsids by the TRIM5alpha restriction factor. Proc Natl Acad Sci USA 103, 5514-5519.
Sun, Q., Fan, W., Chen, K., Dings, X., Chen, S., and Zhong, Q. (2008). Identification of Barkor as a mammalian autophagy-specific factor for Beclin 1 and class III phosphatidylinositol 3-kinase. Proc Natl Acad Sci USA 105, 19211-19216.
Takaesu, G., Kobayashi, T., and Yoshimura, A. (2012). TGFbeta-activated kinase 1 (TAK1)-binding proteins (TAB) 2 and 3 negatively regulate autophagy. Journal of biochemistry 151, 157-166.
Thurston, T. L., Ryzhakov, G., Bloor, S., von Muhlinen, N, and Randow, F. (2009). The TBK1 adaptor and autophagy receptor NDP52 restricts the proliferation of ubiquitin-coated bacteria. Nat Immunol 10, 1215-1221.
Thurston, T. L., Wandel, M. P., von Muhlinen, N., Foeglein, A., and Randow, F. (2012). Galectin 8 targets damaged vesicles for autophagy to defend cells against bacterial invasion. Nature 482, 414-418.
Tomar, D., Singh, R., Singh, A K., and Pandya, C. D. (2012). TRIM13 regulates ER stress induced autophagy and clonogenic ability of the cells. Biochimica et biophysica acta 1823, 316-326.
Versteeg, Gijs A., Rajsbaum, R., Sánchez-Aparicio, Maria T., Maestre, Ana M., Valdiviezo, J., Shi, M., Inn, K.-S., Fernandez-Sesma, A., Jung, J., and García-Sastre, A. (2013). The E3-Ligase TRIM Family of Proteins Regulates Signaling Pathways Triggered by Innate Immune Pattern-Recognition Receptors. Immunity 38, 384-398.
von Muhlinen, N., Thurston, T., Ryzhakov, G., Bloor, S., and Randow, F. (2010). NDPS2, a novel autophagy receptor for ubiquitin-decorated cytosolic bacteria. Autophagy 6, 288-289.
Wei, Y., Pattingre, S., Sinha, S., Bassik, M., and Levine, B. (2008). JNK1-mediated phosphorylation of Bcl-2 regulates starvation-induced autophagy. Mol Cell 30, 678-688.
Wild, P., Farhan, H., McEwan, D. G., Wagner, S., Rogov, V. V., Brady, N. R., Richter, B., Korac, J., Waidmann, O., Choudhary, C., et al. (2011). Phosphorylation of the autophagy receptor optineurin restricts Salmonella growth. Science 333, 228-233.
Yamauchi, K., Wada, K., Tanji, K., Tanaka, M., and Kamitani, T. (2008). Ubiquitination of E3 ubiquitin ligase TRIM5 alpha and its potential role. FEBS J 275, 1540-1555.
Yang, Z., and Klionsky, D. J. (2010). Eaten alive: a history of macroautophagy. Nat Cell Biol 12, 814-822.
Yoshii, S. R., Kishi, C., Ishihara, N., and Mizushima, N. (2011). Parkin mediates proteasome-dependent protein degradation and rupture of the outer mitochondrial membrane. The Journal of biological chemistry 286, 19630-19640.
Youle, R. J., and Narendra, D. P. (2011). Mechanisms of mitophagy. Nat Rev Mol Cell Biol 12, 9-14. Zhang, Y., Yan, L., Zhou, Z., Yang, P., Tian, E., Zhang, K., Zhao, Y., Li, Z., Song, B., Han, J., et al. (2009). SEPA-1 mediates the specific recognition and degradation of P granule components by autophagy in C. elegans. Cell 136, 308-321.

1. A method of treating or reducing the likelihood of the onset of an autophagy-mediated disease in a patient in need thereof comprising administering to said patient an effective amount of a composition comprising an effective amount of an autophagy modulator selected from the group consisting of a neutral lipid, a TRIM protein or a mixtures thereof and optionally, another bioactive agent.

2. The method according to claim 1 wherein said autophagy modulator is a neutral lipid.

3. The method according to claim 1 wherein said autophagy modulator is a TRIM protein.

4. The method according to claim 2 wherein said neutral lipid is effective in enhancing lipid stores and promoting lipid droplets in said patient such that enhancement of autophagy occurs.

5. The method according to claim 2 wherein said neutral lipid is selected from the group consisting of neutral lipids selected from the group consisting of triglycerides, diglycerides, monoglycerides, glycolated mono- or diacylglycerdies, dolichol, polyprenol, polyprenal or very long chain fatty acids.

6. The method according to claim 1 wherein said autophagy modulator is a TRIM protein selected from the group consisting of TRIM5α, TRIM1, TRIM6, TRIM10, TRIM17, TRIM22, TRIM41, TRIM55, TRIM72 and TRIM76, among others (including TRIM 1, TRIM2, TRIM23, TRIM26, TRIM28, TRIM31, TRIM32, TRIM33, TRIM38, TRIM42, TRIM44, TRIM45, TRIM49, TRIM50, TRIM51, TRIM58, TRIM59, TRIM65, TRIM68, TRIM73, TRIM74 and TRIM76 and mixtures thereof.

7. The method according to claim 4 wherein said TRIM protein is TRIM5α.

8. The method according to claim 1 wherein said autophagy modulator is combined with another autophagy modulator selected from the group consisting of flubendazole, hexachlorophene, propidium iodide, bepridil, clomiphene citrate (Z,E), GBR 12909, propafenone, metixene, dipivefrin, fluvoxamine, dicyclomine, dimethisoquin, ticlopidine, memantine, bromhexine, norcyclobenzaprine, diperodon, nortriptyline, tetrachlorisophthalonitrile, phenylmercuric acetate, benzethonium, niclosamide, monensin, bromperidol, levobunolol, dehydroisoandosterone 3-acetate, sertraline, tamoxifen, reserpine, hexachlorophene, dipyridamole, harmaline, prazosin, lidoflazine, thiethylperazine, dextromethorphan, desipramine, mebendazole, canrenone, chlorprothixene, maprotiline, homochlorcyclizine, loperamide, nicardipine, dexfenfluramine, nilvadipine, dosulepin, biperiden, denatonium, etomidate, toremifene, tomoxetine, clorgyline, zotepine, beta-escin, tridihexethyl, ceftazidime, methoxy-6-harmalan, melengestrol, albendazole, rimantadine, chlorpromazine, pergolide, cloperastine, prednicarbate, haloperidol, clotrimazole, nitrofural, iopanoic acid, naftopidil, methimazole, trimeprazine, ethoxyquin, clocortolone, doxycycline, pirlindole mesylate, doxazosin, deptropine, nocodazole, scopolamine, oxybenzone, halcinonide, oxybutynin, miconazole, clomipramine, cyproheptadine, doxepin, dyclonine, salbutamol, flavoxate, amoxapine, fenofibrate, pimethixene, a pharmaceutically acceptable salt thereof and mixtures thereof.

9. The method according to claim 1 wherein said autophagy-mediated disease is cancer, lysosomal storage diseases, Alzheimer's disease, Parkinson's disease; a chronic inflammatory disease, Crohn's disease, diabetes I, diabetes II, metabolic syndrome, an inflammation-associated metabolic disorder, liver disease, renal disease, cardiovascular disease, muscle degeneration and atrophy, symptoms of aging (including the amelioration or the delay in onset or severity or frequency of aging-related symptoms and chronic conditions including muscle atrophy, frailty, metabolic disorders, low grade inflammation, atherosclerosis and associated conditions such as cardiac and neurological both central and peripheral manifestations including stroke, age-associated dementia and sporadic form of Alzheimer's disease, pre-cancerous states, and psychiatric conditions including depression), spinal cord injury, infectious disease and developmental disease.

10. The method according to claim 8 wherein said autophagy-mediated disease is selected from the group consisting of activator deficiency/GM2 gangliosidosis, alpha-mannosidosis, aspartylglucoaminuria, cholesteryl ester storage disease, chronic hexosaminidase A deficiency, cystinosis, Danon disease, Fabry disease, Farber disease, fucosidosis, galactosialidosis, Gaucher Disease (Types I, II and II), GM! Ganliosidosis, including infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs or Wolman disease.

11. The method according to claim 1 wherein said autophagy-mediated disease is selected from the group consisting of Type I and Type II diabetes, severe insulin resistance, hyperinsulinemia, hyperlipidemia, obesity, insulin-resistant diabetes, Mendenhall's Syndrome, Werner Syndrome, leprechaunism, lipoatrophic diabetes, acute and chronic renal insufficiency, end-stage chronic renal failure, glomerulonephritis, interstitial nephritis, pyelonephritis, glomerulosclerosis, GH-deficiency, GH resistance, Turner's syndrome, Laron's syndrome, short stature, increased fat mass-to-lean ratios, decreased CD₄+ T cell counts and decreased immune tolerance, chemotherapy-induced tissue damage, congestive heart failure, Alzheimer's disease, Parkinson's disease, multiple sclerosis, Crohn's disease, peripheral neuropathy, muscular dystrophy, myotonic dystrophy, anorexia nervosa, a viral infection, and a bacterial infection.

12. A pharmaceutical composition comprising an effective amount of a neutral lipid, a TRIM protein or a mixture of a neutral lipid and a TRIM protein, optionally in combination with an additional autophagy modulator, optionally further in combination with at least one additional bioactive agent, in combination with a pharmaceutically acceptable carrier, additive or excipient.

13. The composition according to claim 12 wherein said neutral lipid is selected from the group consisting of neutral lipids selected from the group consisting of triglycerides, diglycerides, monoglycerides, glycolated mono- or diacylglycerdies, dolichol, polyprenol, polyprenal or very long chain fatty acids.

14. The composition according to claim 12 wherein said TRIM protein is selected from the group consisting of TRIM5α, TRIM1, TRIM6, TRIM10, TRIM17, TRIM22, TRIM41, TRIM55, TRIM72 and TRIM76, among others (including TRIM 1, TRIM2, TRIM23, TRIM26, TRIM28, TRIM31, TRIM 32, TRIM33, TRIM38, TRIM42, TRIM44, TRIM45, TRIM49, TRIM50, TRIM51, TRIM58, TRIM59, TRIM65, TRIM68, TRIM73, TRIM74 and TRIM76 and mixtures thereof

15. The composition according to claim 12 wherein said additional autophagy modulator compound selected from the group consisting of flubendazole, hexachlorophene, propidium iodide, bepridil, clomiphene citrate (Z,E), GBR 12909, propafenone, metixene, dipivefrin, fluvoxamine, dicyclomine, dimethisoquin, ticlopidine, memantine, bromhexine, norcyclobenzaprine, diperodon, nortriptyline, tetrachlorisophthalonitrile, phenylmercuric acetate, benzethonium, niclosamide, monensin, bromperidol, levobunolol, dehydroisoandosterone 3-acetate, sertraline, tamoxifen, reserpine, hexachlorophene, dipyridamole, harmaline, prazosin, lidoflazine, thiethylperazine, dextromethorphan, desipramine, mebendazole, canrenone, chlorprothixene, maprotiline, homochlorcyclizine, loperamide, nicardipine, dexfenfluramine, nilvadipine, dosulepin, biperiden, denatonium, etomidate, toremifene, tomoxetine, clorgyline, zotepine, beta-escin, tridihexethyl, ceftazidime, methoxy-6-harmalan, melengestrol, albendazole, rimantadine, chlorpromazine, pergolide, cloperastine, prednicarbate, haloperidol, clotrimazole, nitrofural, iopanoic acid, naftopidil, methimazole, trimeprazine, ethoxyquin, clocortolone, doxycycline, pirlindole mesylate, doxazosin, deptropine, nocodazole, scopolamine, oxybenzone, halcinonide, oxybutynin, miconazole, clomipramine, cyproheptadine, doxepin, dyclonine, salbutamol, flavoxate, amoxapine, fenofibrate, pimethixene, a pharmaceutically acceptable salt thereof and mixtures thereof.

16. The composition according to claim 12 wherein said additional bioactive agent is an additional anticancer agent or a mTOR inhibitor such as pp242, rapamycin, envirolimus, everolimus or cidaforollimus, epigallocatechin gallate (EGCG), caffeine, curcumin or reseveratrol.

17. The composition according to claim 12 wherein said additional bioactive agent is an anticancer agent.

18. The composition according to claim 17 wherein said anticancer agent is selected from the group consisting of Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; gleevec (imatinib); goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; surafenib; talbuvidine (LDT); talc; tamoxifen; tarceva (erlotinib); temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and mixtures thereof.

19. A method of determining whether a patient is at risk for or having an autophagy-related disease state and/or condition comprising measuring the lipid stores and/or lipid droplets in said patient and comparing said measurement with a control of standard, whereby a measurement which is lower than said control or standard is indicative of a patient at risk for or having an autophagy-related disease.

20. (canceled)

21. (canceled)

22. A method of identifying a compound of interest as a potential agent in the treatment of autophagy, said method comprising testing said compound to determine its impact on lipid stores and/or lipid droplets, whereby a compound of interest which increases a lipid store and/or lipid droplets may be identified as a potential autophagy modulator including a drug for reducing the likelihood or treating an autophagy-related disease state or condition.

23. (canceled)

24. (canceled)

25. (canceled)

26. (canceled)

27. (canceled)

28. (canceled)

29. (canceled)

30. (canceled)

31. (canceled)

32. The method of claim 1, comprising administering to the patient:
(a) a pharmaceutically effective amount of at least one neutral lipid selected from the group consisting of triglycerides, diglycerides, monoglycerides, glycolated mono- or diacylglycerdies, dolichol, polyprenol, polyprenal and very long chain fatty acids; and, optionally
(b) at least one additional active ingredient selected from the group consisting of L-carnitine, Acetyl-L-carnitine, a TRIM protein, an anti-cancer agent, an antibiotic, an anti-tuberculosis agent and an antiviral agent.

33. The method of claim 32, wherein:
(a) the autophagy-related disorder is a cancer selected from the group consisting of Stage IV small cell lung cancer, ductal carcinoma in situ, relapsed and refractory multiple myeloma, brain metastases from solid tumors, breast cancer, primary renal cell carcinoma, previously treated renal cell carcinoma, pancreatic cancer, Stage IIb or III adenocarcinoma of the pancreas, non-small cell lung cancer, recurrent advanced non-small cell lung cancer, advanced/recurrent non-small cell lung cancer, metastatic breast cancer, colorectal cancer, metastatic colorectal cancer, unspecified adult solid tumor, α1-antitrypsin deficiency liver cirrhosis, amyotrophic lateral sclerosis and lymphangioleiomyomatosis; and
(b) the additional active ingredient is an autophagy-modulating anti-cancer agent selected from the group consisting of chloroquine, hydrochloroquine, carbamazepine, lithium carbonate and trehalose.

34. (canceled)

35. The method of claim 33, in which a TRIM protein is also co-administered to the patient.

36. (canceled)

37. The pharmaceutical composition of claim 10, comprising:
(a) at least one neutral lipid selected from the group consisting of triglycerides, diglycerides, monoglycerides, glycolated mono- or diacylglycerdies, dolichol, polyprenol, polyprenal and very long chain fatty acids;
(b) at least one TRIM protein;
(c) optionally, an autophagy-modulating anti-cancer agent selected from the group consisting of chloroquine, hydrochloroquine, carbamazepine, lithium carbonate and trehalose;
(d) optionally, one or more compositions selected from the group consisting of the mTOR inhibitor RAD001, gemcitabine, carboplatin, paclitaxel, and bevacizumab, ixabepilone, temsirolimus, sunitinib, vorinostat, MK2206, ABT-263 or abiraterone, docetaxel, sirolimus, vorinostat and bortezomib; and
(e) optionally, at least one pharmaceutically-acceptable excipient.

38. A method of treating or reducing the likelihood of the onset of an autophagy-mediated disease in a patient in need thereof comprising administering to said patient an effective amount of a composition comprising a TRIM protein.

39. The method of claim 36, wherein the TRIM protein is selected from the group consisting of TRIM5α, TRIM1, TRIM6, TRIM10, TRIM17, TRIM22, TRIM41, TRIM55, TRIM72 and TRIM76, and mixtures thereof.

40. The method of claim 36, wherein the TRIM protein is selected from the group consisting of TRIM 1, TRIM2, TRIM23, TRIM26, TRIM28, TRIM31, TRIM 32, TRIM33, TRIM38, TRIM42, TRIM44, TRIM45, TRIM49, TRIM50, TRIM51, TRIM58, TRIM59, TRIM65, TRIM68, TRIM73, TRIM74 and TRIM76 and mixtures thereof.

41. The method of claim 38, comprising administering to the patient at least one additional active ingredient selected from the group consisting of a pharmaceutically effective amount of at least one neutral lipid selected from the group consisting of triglycerides, diglycerides, monoglycerides, glycolated mono- or diacylglycerdies, dolichol, polyprenol, polyprenal and very long chain fatty acids, L-carnitine, Acetyl-L-carnitine, an anti-cancer agent, an antibiotic, an anti-tuberculosis agent and an antiviral agent.

42. The method of claim 41, wherein:
(a) the autophagy-mediated disorder is a cancer selected from the group consisting of Stage IV small cell lung cancer, ductal carcinoma in situ, relapsed and refractory multiple myeloma, brain metastases from solid tumors, breast cancer, primary renal cell carcinoma, previously treated renal cell carcinoma, pancreatic cancer, Stage IIb or III adenocarcinoma of the pancreas, non-small cell lung cancer, recurrent advanced non-small cell lung cancer, advanced/recurrent non-small cell lung cancer, metastatic breast cancer, colorectal cancer, metastatic colorectal cancer, unspecified adult solid tumor, al-antitrypsin deficiency liver cirrhosis, amyotrophic lateral sclerosis and lymphangioleiomyomatosis; and
(b) the additional active ingredient is an autophagy-modulating anti-cancer agent selected from the group consisting of chloroquine, hydrochloroquine, carbamazepine, lithium carbonate and trehalose.

43.-47. (canceled)


SEQUENCE LISTING (16 sequences)

SEQ ID NO: 1 - DNA, 20 bp, Artificial Sequence
  Actin-F
aagacctgtacgccaacaca

SEQ ID NO: 2 - DNA, 20 bp, Artificial Sequence
  Actin-R
tgatctccttctgcatcctg

SEQ ID NO: 3 - DNA, 20 bp, Artificial Sequence
  DGAT1-F
tcaagtatggcatcctggtg

SEQ ID NO: 4 - DNA, 20 bp, Artificial Sequence
  DGAT1-R
aagacattggccgcaataac

SEQ ID NO: 5 - DNA, 20 bp, Artificial Sequence
  DGAT2-F
tccagctggtgaagacacac

SEQ ID NO: 6 - DNA, 20 bp, Artificial Sequence
  DGAT2-R
tgtgctgaagttgcagaagg

SEQ ID NO: 7 - DNA, 20 bp, Artificial Sequence
  PNPLA1-F
ccagatagaactcgcccttg

SEQ ID NO: 8 - DNA, 20 bp, Artificial Sequence
  PNPLA1-R
gtgaggttgtgtggctcctt

SEQ ID NO: 9 - DNA, 20 bp, Artificial Sequence
  PNPLA2-F
caacaccagcatccagttca

SEQ ID NO: 10 - DNA, 20 bp, Artificial Sequence
  PNPLA2-R
atccctgcttgcacatctct

SEQ ID NO: 11 - DNA, 20 bp, Artificial Sequence
  PNPLA3-F
atgtccaccagctcatctcc

SEQ ID NO: 12 - DNA, 20 bp, Artificial Sequence
  PNPLA3-R
gcatccacgacttcgtcttt

SEQ ID NO: 13 - DNA, 20 bp, Artificial Sequence
  PNPLA4-F
agaaccgactgcacgtatcc

SEQ ID NO: 14 - DNA, 20 bp, Artificial Sequence
  PNPLA4-R
tgctggctaggaggacctta

SEQ ID NO: 15 - DNA, 20 bp, Artificial Sequence
  PNPL5-F
tcctggggctcatatgtctc

SEQ ID NO: 16 - DNA, 20 bp, Artificial Sequence
  PNPLA5-R
agtccacgtctctccaggaa